(19) United States
(12) Patent Application Publication
(10) Pub. No.: US 2025/0163168 A1
(43) Pub. Date: May 22, 2025

(54) NOVEL ANTIBODIES AND ANTIBODY CONJUGATES FOR THE TREATMENT OF METABOLIC DISORDERS

(71) Applicant: PFIZER INC., NEW YORK, NY (US)

(72) Inventors: James Reasoner APGAR, Newton, MA (US); Robert Gregory DULLEA, Groton, MA (US); Jean-Philippe FORTIN, Roslindale, MA (US); Allison Nicole LAU, Newton, MA (US); Laura LIN LOHSE, Weston, MA (US); Weixu MENG, San Diego, CA (US); Lidia MOSYAK, Newton, MA (US)

(73) Assignee: PFIZER INC., NEW YORK, NY (US)

(21) Appl. No.: 18/947,069

(22) Filed: Nov. 14, 2024

Related U.S. Application Data
(60) Provisional application No. 63/600,487, filed on Nov. 17, 2023.
(60) Provisional application No. 63/718,764, filed on Nov. 11, 2024.

(51) Int. Cl. C07K 16/28; A61K 39/00; A61P 3/04

(52) U.S. Cl. CPC C07K 16/2869; A61P 3/04; A61K 2039/505; C07K 2317/24; C07K 2317/33; C07K 2317/75; C07K 2317/76; C07K 2317/92; C07K 2317/94

(57) ABSTRACT

Disclosed herein are novel antibodies, novel agonist peptides, and novel dual agonist peptides. Further disclosed herein are novel antibody conjugates comprising the novel antibodies, novel agonist peptides, and/or novel dual agonist peptides disclosed herein. In some embodiments, the antibodies or antibody conjugates of the invention may be used to treat metabolic diseases.

### SEQUENCE LISTING

The instant application contains a Sequence Listing which has been submitted electronically in ASCII format and is hereby incorporated by reference in its entirety. Said ASCII copy, created on Nov. 11, 2024, is named PC073040A.txt and is 64,151 bytes in size.

### BACKGROUND

The present invention relates to antibodies that bind to glucose-dependent insulinotropic polypeptide receptor (GIPR). The present invention further relates to GIPR antibody conjugates comprising a GIPR antibody conjugated to one more additional moieties. In some embodiments, the GIPR antibody can be conjugated to a glucagon-like peptide-1 (GLP-1) receptor (GLP-1R) agonist and/or a glucagon receptor (GCGR) agonist. The present invention also pertains to related molecules, for example, nucleic acid encoding the GIPR antibodies disclosed herein, GLP-1R agonist peptides, GCGR agonist peptides, pharmaceutical compositions, methods of using the compositions disclosed herein, and their use in diagnostics and therapeutics.

### SUMMARY OF THE INVENTION

The present disclosure provides antibodies that bind to GIPR, and in particular antibodies that bind to the extra cellular domain of GIPR, uses of the antibodies and associated methods, and the processes for making, preparing, and producing these antibodies. Antibodies of the disclosure are useful in one or more of diagnosis, prophylaxis, or treatment of disorders or conditions mediated by, or associated with GIPR activity, including, but not limited to metabolic diseases including but not limited to obesity and diabetes.

The present disclosure also provides anti-GIPR antibody conjugates wherein the antibody is conjugated to a GLP-1R agonist or a dual agonist of GLP1-R and GCGR. Also provided are novel GLP-1R agonists and dual agonists of GLP-1R and GCGR.

Polynucleotides encoding antibodies are provided. Host cells that express the antibodies are provided. Methods of treatment using the antibodies and antibody conjugates are provided.

In some embodiments, disclosed herein is an isolated antibody that binds to human gastric inhibitory polypeptide receptor (GIPR), comprising a heavy chain and a light chain, and the heavy chain comprises a heavy chain variable region (VH) and the light chain comprises a light chain variable region (VL), wherein: (i) the VH comprises: a complementary determining region (CDR)-H1 comprising an amino acid sequence of SEQ ID NO: 15, SEQ ID NO: 16, or SEQ ID NO: 17; a VH CDR-H2 comprising an amino acid sequence of SEQ ID NO: 18 or SEQ ID NO: 19; and a VH CDR-H3 comprising an amino acid sequence of SEQ ID NO: 20; and (ii) the VL comprises a CDR-L1 comprising an amino acid sequence of SEQ ID NO: 23; a VL CDR-L2 comprising an amino acid sequence of SEQ ID NO: 24; and a VL CDR-L3 comprising an amino acid sequence of SEQ ID NO: 25.

In some embodiments, disclosed herein is an isolated antibody that binds to human gastric inhibitory polypeptide receptor (GIPR), comprising a heavy chain and a light chain, wherein the heavy chain comprises a heavy chain variable region (VH) and the light chain comprises a light chain variable region (VL), and wherein: (i) the VH comprises a VH complementary determining region (CDR)-H1 comprising an amino acid sequence of SEQ ID NO: 2, SEQ ID NO: 3, or SEQ ID NO: 4; a VH CDR-H2 comprising an amino acid sequence of SEQ ID NO: 5 or SEQ ID NO: 6; and a VH CDR-H3 comprising an amino acid sequence of SEQ ID NO: 7; and (ii) the VL comprises a VL CDR-L1 comprising an amino acid sequence of SEQ ID NO: 10; a VL CDR-L2 comprising an amino acid sequence of SEQ ID NO: 11; and a VL CDR-L3 comprising an amino acid sequence shown in SEQ ID NO: 12.

In some embodiments, disclosed herein is an isolated antibody comprising a light chain comprising an amino acid sequence of SEQ ID NO: 8; and a heavy chain comprising an amino acid sequence of SEQ ID NO: 13. In another embodiment, disclosed herein is an isolated antibody comprising a light chain comprising an amino acid sequence of SEQ ID NO: 21; and a heavy chain comprising an amino acid sequence of SEQ ID NO: 26.

In some embodiments, disclosed herein is a pharmaceutical composition comprising an isolated antibody described herein; and a pharmaceutically acceptable carrier.

In some embodiments, disclosed herein is a method of treating a condition comprising administering to a subject in need thereof a therapeutically effective amount of an isolated antibody described herein or an antibody conjugate described herein.

### BRIEF DESCRIPTION OF THE DRAWINGS

FIG. 1 depicts the effect of anti-GIPR antibodies GIPR-0107 and GIPR-2767 on blood glucose during Oral Glucose Tolerance Test.

FIG. 2 depicts the effect of chronic administration of anti-GIPR antibodies GIPR-0107 and GIPR-2767 on body weight shown as percent change from baseline.

FIG. 3 depicts the effects of anti-GIPR antibody GIPR-2767 at 0.1 mpk, 0.3 mpk, 1 mpk, and 3 mpk dose levels on blood glucose during an Oral Glucose Tolerance Test (OGTT).

FIG. 4 depicts the effect of anti-GIPR antibody GIPR-2767 at 0.1 mpk, 0.3 mpk, 1 mpk, and 3 mpk dose levels on body weight shown as percentage change from baseline over a 6 week period.

FIG. 5A depicts the effect of anti-GIPR antibodies 2G10, GIPR-0107, and GIPR-2767 at 1 mpk on blood glucose during an Oral Glucose Tolerance Test (OGTT). FIG. 5B depicts the effect of anti-GIPR antibodies 2G10, GIPR-0107, and GIPR-2767 at 1 mpk on blood glucose AUC₀₋₁₂₀ min.

FIG. 6 depicts the effect of chronic GIPR-2767 administration on body weight (% change from baseline) and food intake.

FIG. 7 illustrates the unique binding site residues between GIPR-0107, GIPR-2767, 2G10, and GIPG013.

FIG. 8 shows a model of anti-GIPR antibodies blocking GIPR/GIP interactions.

### DETAILED DESCRIPTION

The present invention may be understood more readily by reference to the following detailed description of the embodiments of the invention and the Examples included herein. It is to be understood that this invention is not limited to specific methods of making that may of course vary. It is to be also understood that the terminology used herein is for the purpose of describing specific embodiments only and is not intended to be limiting.

Glucose-dependent insulinotropic polypeptide (GIP, formerly called gastric inhibitory polypeptide) is a 42-amino acid peptide secreted from K-cells in the small intestine (duodenum and jejunum). Human GIP is derived from the processing of proGIP, a 153-amino acid precursor encoded by a gene localized on chromosome 17 (See e.g., Inagaki et al., Mol Endocrinol 1989; 3:1014-1021; and Fehmann et al. Endocr Rev. 1995; 16:390-410). GIP secretion is induced by food ingestion. GIP is a known insulinotropic factor (or “incretin”) that enhances glucose-dependent insulin secretion. GIP has additional physiological effects in multiple tissues, including the promotion of fat storage in the adipose. Intact GIP is rapidly inactivated by dipeptidyl peptidase 4 (DPPIV).

GIPR belongs to the glucagon subfamily of class B1 G protein-coupled receptors (GPCRs) characterized by an extracellular N-terminal domain, seven transmembrane domains and an intracellular C-terminus (See e.g. Zhao et al. Nat Commun. 2022, 13:1057). The N-terminal extracellular domain forms the primary peptide recognition and binding site of the receptor. Upon stimulation with GIP, GIPR undergoes structural changes from inactive to active conformations, thereby triggering a Gas-mediated increase in cAMP production. GIPR is expressed in various tissues, including the pancreas, gut, adipose tissue, vasculature, heart, and brain (see e.g. Hammoud et al. Nat Rev Endocrinol 2023; 18: 201-216). Human GIPR comprises 466 amino acids and is encoded by a gene located on chromosome 19 (see e.g. Gremlich et al., Diabetes. 1995; 44:1202-8; and Volz et al., FEBS Lett. 1995, 373:23-29). Studies suggest that alternative mRNA splicing results in the production of GIPR variants with differing length (see e.g., Harada et al. Am J Physiol Endocrinol Metab. 2008. 294: E61-E68; and Marti-Solano et al. Nature. 2020, 587: 650-656)

GIPR knockout mice are resistant to high fat diet-induced weight gain and have improved insulin sensitivity and lipid profiles (see e.g. Yamada et al. Diabetes. 2006, 55:S86; and Miyawaki et al. Nature Med. 2002, 8:738-742). Recent data supports that heterozygous loss of function in GIPR results in lower BMI and obesity risk in humans (see e.g. Akbari et al. Science. 2021, 373: 6550). Small molecules, peptides, and monoclonal antibodies with antagonist activity at GIPR have been shown to prevent weight gain and insulin resistance in preclinical obesity models (see e.g. Nakamura et al. Diabetes Metab Syndr Obes. 2021, 14:1095-1105; Yang et al. Mol Metab. 2022, 66: 101638; and Killion et al. 2018). The combination of GIPR modulators with GLP-1R agonists has been associated with superior weight loss (see e.g. Lu et al. Cell Rep Med. 2021, 2(5):100263). Collectively, these links to obesity and metabolic diseases suggest that GIPR inhibition is a useful approach for therapeutic intervention, both as monotherapy and in combination with other agents including GLP-1R agonists. Moreover, human epicardial adipose tissue—which plays a crucial role in the development and progression of coronary artery disease, atrial fibrillation, and heart failure—has been found to express GIPR genes and proteins. See e.g. Malavazos et al., European Journal of Preventive Cardiology (2023) 00, 1-14.

There continues to be a need for alternative GIPR antagonists, for example, for developing new and/or improved pharmaceuticals (e.g., more effective, more selective, less toxic, improved patient compliance, and/or having improved biopharmaceutical properties such as physical stability; solubility; oral bioavailability; appropriate metabolic stability; clearance; half-life) to treat or prevent GIPR-related conditions, diseases, or disorders, such as those described herein. The present invention is directed to these and other important ends.

The techniques and procedures described or referenced herein are generally well understood and commonly employed using conventional methodology by those skilled in the art, such as, for example, the widely utilized methodologies described in Sambrook et al, Molecular Cloning: A Laboratory Manual 3rd. edition (2001) Cold Spring Harbor Laboratory Press, Cold Spring Harbor, N.Y. CURRENT PROTOCOLS IN MOLECULAR BIOLOGY (F. M. Ausubel, et al. eds., (2003)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds); Gene Transfer Vectors for Mammalian Cells (J. M. Miller and M. P. Calos, eds., 1987); PCR: The Polymerase Chain Reaction, (Mullis et al, eds., 1994); Current Protocols in Immunology (J. E. Coligan et al, eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999)); The Antibodies (M. Zanetti and J. D. Capra, eds., Harwood Academic Publishers, 1995); and updated versions thereof.

### Definitions

Unless otherwise defined herein, scientific and technical terms used in connection with the present invention have the meanings that are commonly understood by those of ordinary skill in the art.

As used herein, the singular form “a”, “an”, and “the” include plural references unless indicated otherwise. For example, “an” antibody includes one or more antibodies.

Where aspects or embodiments of the invention are described in terms of a Markush group or other grouping of alternatives, the present invention encompasses not only the entire group listed as a whole, but each member of the group individually and all possible subgroups of the main group, but also the main group absent one or more of the group members. The present invention also envisages the explicit exclusion of one or more of any of the group members in the claimed invention.

Any example(s) following the term “e.g.” or “for example” is not meant to be exhaustive or limiting.

As used herein, the term “about” when used to modify a numerically defined parameter (e.g., the dose of an antibody or antibody conjugate disclosed herein) means that the parameter may vary by as much as 10% below or above the stated numerical value for that parameter. For example, a dose of about 5 mg means 5%±10%, i.e. it may vary between 4.5 mg and 5.5 mg.

An “antibody” refers to an immunoglobulin molecule capable of specific binding to a target, such as a polypeptide, carbohydrate, polynucleotide, lipid, etc., through at least one antigen binding domain, located in the variable region of the immunoglobulin molecule. As used herein, the term “antibody” can encompass any type of antibody (e.g. monospecific, bispecific), and includes portions of intact antibodies that retain the ability to bind to a given antigen (e.g. an “antigen-binding fragment”), and any other modified configuration of an immunoglobulin molecule that comprises an antigen binding domain. An exemplary antibody comprises i) a variable region of the light chain, heavy chain or both and ii) a constant region of the heavy chain comprising three sequential immunoglobulin domains (CH1, CH2, and CH3) and of the light chain comprising a single immunoglobulin domain (CL). In some embodiments, an antibody can encompass an antibody conjugated to a secondary agent, i.e., an antibody conjugate.

An antibody or antibody conjugate includes an antibody of any class, such as IgG, IgA, or IgM (or a sub-class thereof), and the antibody need not be of any particular class. Depending on the antibody amino acid sequence of the constant region of its heavy chains (HC), immunoglobulins can be assigned to different classes. There are five major classes of immunoglobulins: IgA, IgD, IgE, IgG, and IgM, and several of these may be further divided into subclasses (isotypes), e.g., IgG₁, IgG₂, IgG₃, IgG₄, IgA₁ and IgA₂. The heavy chain constant regions that correspond to the different classes of immunoglobulins are called alpha, delta, epsilon, gamma, and mu, respectively. The subunit structures and three-dimensional configurations of different classes of immunoglobulins are well known.

Examples of antibody antigen-binding fragments and modified configurations include (i) a Fab fragment (a monovalent fragment consisting of the VL, VH, CL and CH1 domains); (ii) a F(ab′)2 fragment (a bivalent fragment comprising two Fab fragments linked by a disulfide bridge at the hinge region); and (iii) a Fv fragment consisting of the VL and VH domains of a single arm of an antibody. Furthermore, although the two domains of an Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., BIRD, R. E., et al., “Single-Chain Antigen-Binding Proteins,” Science, 1988, 242(4877):423-442 and HUSTON, J. S., et al., “Protein engineering of antibody binding sites: recovery of specific activity in an anti-digoxin single-chain Fv analogue produced in Escherichia coli,” Proceedings of the National Academy of Science, 1988, 85(16):5879-5883. Other forms of single chain antibodies, such as diabodies are also encompassed.

In addition, further encompassed are antibodies that are missing a C-terminal lysine (K) amino acid residue on a heavy chain polypeptide (e.g. human IgG1 heavy chain comprises a terminal lysine). As is known in the art, the C-terminal lysine is sometimes clipped during antibody production, resulting in an antibody with a heavy chain lacking the C-terminal lysine. Alternatively, an antibody heavy chain may be produced using a nucleic acid that does not include a C-terminal lysine.

Further encompassed are antibodies that have post-translational modifications at one or more amino acids in one or both of the heavy chain and light chain.

An “IgG1”, “IgG2”, “IgG3” and “IgG4” antibody” refers to an antibody that
(i) has a first and a second heavy chains same or different and comprising a first VH and a second VH respectively, has a first and second light chains same or different comprising a first VL and a second VL respectively, the fist VH and the first VL form a first binding domain and the second VH and second VL form a second binding domain, and the first binding domain and the second binding domain bind to the same target or two different targets,
(ii) each heavy chain has a constant region that has an amino acid sequence that is at least 95% identical to (a) a wildtype IgG1 heavy chain constant region having the amino acid sequence; (b) a wildtype IgG2 heavy chain constant region having the amino acid sequence; (c) a wildtype IgG3 heavy chain constant region having the amino acid sequence; and (d) a wildtype IgG4 heavy chain constant region having the amino acid sequence, respectively.

An “IgG antibody” refers to an antibody that is an IgG2, IgG2, IgG3 or IgG4 antibody.

An IgG CL refers to a light chain constant region that has an amino acid sequence that is at least 95% identical to a wildtype IgG light chain constant region.

An IgG hinge refers to a polypeptide having an amino acid sequence that is at least 95 identical to a wildtype IgG hinge region sequence.

An IgG CH1, IgG Fc chain is similarly defined.

An IgG1, 2, 3 and 4 light chain constant region (CL), an IgG1, 2, 3, 4 CH1, an IgG1, 2, 3 and 4 hinge, an IgG1, 2, 3 and 4 Fc chain is similarly defined.

A “full length IgG1 antibody” refers to an IgG1 antibody as described above, and the first and the second light chain further comprising a first light chain constant region and a second light chain constant region respectively, and each of the first light chain constant region and the second light chain constant region has an amino acid sequence that is 95% identical to one of the wildtype IgG1 light chain constant region. A “full length IgG1 antibody” is similarly defined. A full length IgG antibody is a full length IgG1, IgG2, IgG3 or IgG4 antibody.

Examples of antibody antigen-binding fragments and modified configurations include (i) a Fab fragment (a monovalent fragment consisting of the VL, VH, CL and CH1 domains); (ii) a F(ab′)2 fragment (a bivalent fragment comprising two Fab fragments linked by a disulfide bridge at the hinge region); and (iii) a Fv fragment consisting of the VL and VH domains of a single arm of an antibody. Furthermore, although the two domains of an Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv)); see e.g., Bird et al., Science 1988; 242:423-426 and Huston et al., Proc. Natl. Acad. Sci. 1988 USA 85:5879-5883. Other forms of single chain antibodies, such as diabodies are also encompassed.

In addition, further encompassed are antibodies that are missing a C-terminal lysine (K) amino acid residue on a heavy chain polypeptide (e.g. human IgG1 heavy chain comprises a terminal lysine). As is known in the art, the C-terminal lysine is sometimes clipped during antibody production, resulting in an antibody with a heavy chain lacking the C-terminal lysine. Alternatively, an antibody heavy chain may be produced using a nucleic acid that does not include a C-terminal lysine.

A “variable region” of an antibody refers to the variable region of the antibody light chain or the variable region of the antibody heavy chain, either alone or in combination. As known in the art, the variable regions of the heavy and light chains each consist of four framework regions (FRs) connected by three complementarity determining regions (CDRs) also known as hypervariable regions, and contribute to the formation of the antigen binding site of antibodies. If variants of a subject variable region are desired, particularly with substitution in amino acid residues outside of a CDR region (i.e., in the framework region), appropriate amino acid substitution, preferably, conservative amino acid substitution, can be identified by comparing the subject variable region to the variable regions of other antibodies which contain CDR1 and CDR2 sequences in the same canonical class as the subject variable region (Chothia and Lesk, J Mol Biol 196(4): 901-917, 1987).

In certain embodiments, definitive delineation of a CDR and identification of residues comprising the binding site of an antibody is accomplished by solving the structure of the antibody or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. In certain embodiments, various methods of analysis can be employed to identify or approximate the CDR regions. Examples of such methods include, but are not limited to, the Kabat definition, the Chothia definition, the AbM definition, the contact definition, the extended definition, and the conformational definition.

The Kabat definition is a standard for numbering the residues in an antibody and is typically used to identify CDR regions. See, e.g., The Chothia definition is similar to the Kabat definition, but the Chothia definition considers positions of certain structural loop regions. See, e.g., CHOTHIA, C. and LESK, A. M., “Canonical structures for the hypervariable regions of immunoglobulins,” Journal of Molecular Biology, 1986, 196(4):901-917. The extended definition is the combination of the Kabat and Chothia definitions. The AbM definition uses an integrated suite of computer programs produced by Oxford Molecular Group that model antibody structure. See, e.g., MARTIN, A. C., et al., “Modeling antibody hypervariable loops: a combined algorithm,” Proceedings of the National Academy of Sciences, 1989, 86(23):9268-9272 and “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by SAMUDRALA, R., et al., 1999, “Ab Initio Protein Structure Prediction Using a Combined Hierarchical Approach,” PROTEINS: Structure, Function and Genetics Supplement, 1999, 3:194-. The contact definition is based on an analysis of the available complex crystal structures. See, e.g., MACCALLUM, R. M., et al., “Antibody-antigen Interactions: Contact Analysis and Binding Site Topography,” Journal of Molecular Biology, 1996, 262(5):732-745, In another approach, referred to herein as the “conformational definition” of CDRs, the positions of the CDRs may be identified as the residues that make enthalpic contributions to antigen binding. See, e.g., MAKABE, K., et al., “Thermodynamic Consequences of Mutations in Vernier Zone Residues of a Humanized Anti-human Epidermal Growth Factor Receptor Murine Antibody, 528,” Journal of Biological Chemistry, 2008, 283(2):1156-1166.

The Pfabat numbering method is a defined algorithm for consistent antibody numbering, based on the Kabat numbering system (Sequences of Proteins of Immunological Interest, Fifth Edition by Kabat et al., NIH Publication NO: 91-3242, 1991). Unlike many other computational implementations of Kabat numbering, Pfabat numbers entire human IgG1 heavy and light chains, including the constant (C) regions and heavy chain hinge. The Pfabat numbering method is further described, for example, in International Publication WO/2023/166418 (published 7 Sep. 2023), which is hereby incorporated by reference for all purposes.

Still other CDR boundary definitions may not strictly follow one of the above approaches, but will nonetheless overlap with at least a portion of the Kabat CDRs, although they may be shortened or lengthened in light of prediction or experimental findings that particular residues or groups of residues do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any one or more of Kabat, Chothia, extended, AbM, contact, or conformational definitions, or Pfabat method.

A “constant region” of an antibody refers to the constant region of the antibody light chain or the constant region of the antibody heavy chain, either alone or in combination. An IgG heavy chain constant region contains three sequential immunoglobulin domains (CH1, CH2, and CH3), with a hinge region between the CH1 and CH2 domains. An IgG light chain constant region contains a single immunoglobulin domain (CL)

A “Fc domain” refers to the portion of an immunoglobulin (Ig) molecule that correlates to a crystallizable fragment obtained by papain digestion of an Ig molecule. As used herein, the term relates to the 2-chained constant region of an antibody, each chain excluding the first constant region immunoglobulin domain. Within an Fc domain, there are two “Fc chains” (e.g. a “first Fc chain” and a “second Fc chain”). “Fc chain” generally refers to the C-terminal portion of an antibody heavy chain. Thus, Fc chain refers to the last two constant region immunoglobulin domains (CH2 and CH3) of IgA, IgD, and IgG heavy chains, and the last three constant region immunoglobulin domains of IgE and IgM heavy chains, and optionally the flexible hinge N-terminal to these domains.

Although the boundaries of the Fc chain may vary, the human IgG heavy chain Fc chain is usually defined to comprise residues C226 or P230 to its carboxyl-terminus, wherein the numbering is according to the EU index of Edelman et al., Proc. Natl. Acad. Sci. USA 1969; 63(1):78-85 and as described in Kabat et al., 1991. Typically, the Fc chain comprises from about amino acid residue 236 to about 447 of the human IgG1 heavy chain constant region. “Fc chain” may refer to this polypeptide in isolation, or in the context of a larger molecule (e.g. in an antibody heavy chain or Fc fusion protein).

A “functional” Fc domain refers to an Fc domain that possesses at least one effector function of a native sequence Fc domain. Exemplary “effector functions” include C1q binding; complement dependent cytotoxicity (CDC); Fc receptor binding; antibody-dependent cell-mediated cytotoxicity (ADCC); phagocytosis; down-regulation of cell surface receptors (e.g., B cell receptor); and B cell activation, etc. Such effector functions generally require the Fc domain to be combined with a binding domain (e.g., an antibody variable region) and can be assessed using various assays known in the art for evaluating such antibody effector functions.

A “native sequence” Fc chain refers to a Fc chain that comprises an amino acid sequence identical to the amino acid sequence of an Fc chain found in nature. A “variant” Fc chain comprises an amino acid sequence which differs from that of a native sequence Fc chain by virtue of at least one amino acid modification

A “monoclonal antibody” (mAb) refers to an antibody that is derived from a single copy or clone, including e.g., any eukaryotic, prokaryotic, or phage clone. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler and Milstein, 1975, Nature 256:495, or may be made by recombinant DNA methods such as described in U.S. Pat. No. 4,816,567. In another example, monoclonal antibodies may be isolated from phage libraries such as those generated using the techniques described in McCafferty et al., 1990, Nature 348:552-554.

A “human antibody” refers to an antibody which possesses an amino acid sequence which corresponds to that of an antibody produced by a human or has been made using any technique for making fully human antibodies. For example, fully human antibodies may be obtained by using commercially available mice that have been engineered to express specific human immunoglobulin proteins, or by library (e.g. phage, yeast, or ribosome) display techniques for preparing fully human antibodies. This definition of a human antibody specifically excludes a humanized antibody comprising non-human antigen binding residues.

A “chimeric antibody” refers to an antibody in which the variable region sequences are derived from one species and the constant region sequences are derived from another species, such as an antibody in which the variable region sequences are derived from a mouse antibody and the constant region sequences are derived from a human antibody.

A “humanized” antibody refers to a non-human (e.g. murine) antibody that is a chimeric antibody that contains minimal sequence derived from non-human immunoglobulin. Preferably, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. The humanized antibody may comprise residues that are found neither in the recipient antibody nor in the imported CDR or framework sequences, but are included to further refine and optimize antibody performance.

An “antigen” refers to the molecular entity used for immunization of an immunocompetent vertebrate to produce the antibody that recognizes the antigen or to screen an expression library (e.g., phage, yeast or ribosome display library, among others) for antibody selection. Herein, antigen is termed more broadly and is generally intended to include target molecules that are specifically recognized by the antibody, thus including fragments or mimics of the molecule used in an immunization process for raising the antibody or in library screening for selecting the antibody.

An “epitope” refers to the area or region of an antigen to which an antibody specifically binds, e.g., an area or region comprising residues that interact with the antibody, as determined by any method well known in the art. There are many methods known in the art for mapping and characterizing the location of epitopes on proteins, including solving the crystal structure of an antibody-antigen complex, competition assays, gene fragment expression assays, epitope mapping, and synthetic peptide-based assays, as described, for example, in Chapter 11 of Harlow and Lane, Using Antibodies, a Laboratory Manual, Cold Spring Harbor Laboratory Press, Cold Spring Harbor, New York, 1999. In addition or alternatively, during the discovery process, the generation and characterization of antibodies may elucidate information about desirable epitopes. From this information, it is then possible to competitively screen antibodies for binding to the same epitope.

In addition, the epitope to which an antibody binds can be determined in a systematic screening by using overlapping peptides derived from the antigen and determining binding by the antibody. According to the gene fragment expression assays, the open reading frame encoding the antigen can be fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled antigen fragments is then determined by immunoprecipitation and gel electrophoresis.

Certain epitopes can also be identified by using large libraries of random peptide sequences displayed on the surface of phage particles (phage libraries) or yeast (yeast display). Alternatively, a defined library of overlapping peptide fragments can be tested for binding to the test antibody in simple binding assays. In an additional example, mutagenesis of an antigen, domain swapping experiments and alanine scanning mutagenesis can be performed to identify residues required, sufficient, or necessary for epitope binding.

At its most detailed level, the epitope for the interaction between the antigen and the antibody can be defined by the spatial coordinates defining the atomic contacts present in the antigen-antibody interaction, as well as information about their relative contributions to the binding thermodynamics. At a less detailed level, the epitope can be characterized by the spatial coordinates defining the atomic contacts between the antigen and antibody. At a further less detailed level the epitope can be characterized by the amino acid residues that it comprises as defined by a specific criterion, e.g., by distance between atoms (e.g., heavy, i.e., non-hydrogen atoms) in the antibody and the antigen. At a further less detailed level the epitope can be characterized through function, e.g., by competition binding with other antibodies. The epitope can also be defined more generically as comprising amino acid residues for which substitution by another amino acid will alter the characteristics of the interaction between the antibody and antigen (e.g. using alanine scanning).

From the fact that descriptions and definitions of epitopes, dependent on the epitope mapping method used, are obtained at different levels of detail, it follows that comparison of epitopes for different antibodies on the same antigen can similarly be conducted at different levels of detail.

Epitopes described at the amino acid level, e.g., determined from an X-ray crystallography, Nuclear Magnetic Resonance (NMR) spectroscopy, hydrogen/deuterium exchange Mass Spectrometry (H/D-MS), are said to be identical if they contain the same set of amino acid residues. Epitopes are said to overlap if at least one amino acid is shared by the epitopes. Epitopes are said to be separate (unique) if no amino acid residue is shared by the epitopes.

Yet another method which can be used to characterize an antibody is to use competition assays with other antibodies known to bind to the same antigen, to determine if an antibody of interest binds to the same epitope as other antibodies. Competition assays are well known to those of skill in the art. Epitopes characterized by competition binding are said to be overlapping if the binding of the corresponding antibodies are mutually exclusive, i.e., binding of one antibody excludes simultaneous or consecutive binding of the other antibody. The epitopes are said to be separate (unique) if the antigen is able to accommodate binding of both corresponding antibodies simultaneously.

Epitopes can be linear or conformational. In a linear epitope, all of the points of interaction between the protein and the interacting molecule (such as an antibody) occur linearly along the primary amino acid sequence of the protein. A “nonlinear epitope” or “conformational epitope” comprises noncontiguous polypeptides (or amino acids) within the antigenic protein to which an antibody specific to the epitope binds.

The term “binding affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art. Low-affinity antibodies generally bind antigen slowly and tend to dissociate readily, whereas high-affinity antibodies generally bind antigen faster and tend to remain bound longer. In particular, the term “binding affinity” is intended to refer to the dissociation rate of a particular antigen-antibody interaction. The K_{D} is the ratio of the rate of dissociation, also called the “off-rate (k_{off})” or “k_{d}” to the association rate, or “on-rate (kₒₙ)” or “kₐ”. Thus, K_{D} equals k_{off}/kₒₙ(or k_{d}/kₐ) and is expressed as a molar concentration (M). It follows that the smaller the K_{D}, the stronger the affinity of binding. Therefore, a K_{D} of 1 μM indicates weaker binding affinity compared to a K_{D} of 1 nM. K_{D} values for antibodies can be determined using methods well established in the art. One exemplary method for determining the K_{D} of an antibody is by using surface plasmon resonance (SPR), typically using a biosensor system such as BIACORE system. BIACORE kinetic analysis comprises analyzing the binding and dissociation of an antigen from chips with immobilized molecules (e.g., molecules comprising epitope binding domains), on their surface. Another method for determining the K_{D} of an antibody is by using Bio-Layer Interferometry, typically using OCTET® technology (Octet QK^{e} system, ForteBio). Alternatively, or in addition, a KinExA (Kinetic Exclusion Assay) assay, available from Sapidyne Instruments (Boise, ID) can also be used.

A “monospecific antibody” refers to an antibody that comprises one or more antigen binding sites per molecule such that any and all binding sites of the antibody specifically recognize the identical epitope on the antigen. Thus, in cases where a monospecific antibody has more than one antigen binding site, the binding sites compete with each other for binding to one antigen molecule.

A “bispecific antibody” refers to a molecule that has binding specificity for at least two different epitopes. In some embodiments, bispecific antibodies can bind simultaneously two different antigens. In other embodiments, the two different epitopes may reside on the same antigen.

An “antibody conjugate” refers to an antibody chemically linked to a secondary agent. In some embodiments, the active agent is a small molecule compound. In some embodiments, the active agent is a peptide. In some embodiments, an antibody is chemically linked to a secondary agent by a chemical linker. In some embodiments, an antibody is chemically linked to a secondary agent by a peptide.

The term “K_{b}” refers to an equilibrium dissociation constant for a competitive antagonist, which is the molar concentration that would occupy half of the receptors at equilibrium.

The term “half maximal effective concentration (EC₅₀)” refers to the concentration of a therapeutic agent which causes a response halfway between the baseline and maximum after a specified exposure time. The EC₅₀ value is commonly used, and is used herein, as a measure of potency.

The term “half maximal inhibitory concentration (IC₅₀)” refers to the concentration of a therapeutic agent that is needed to inhibit a given biological process, function or component by 50%. An IC₅₀ is a measure of potency of a substance (e.g., a therapeutic agent) in inhibiting a specific biological process, function or component.

An “agonist” refers to a substance which promotes (i.e., induces, causes, enhances, or increases) the biological activity or effect of another molecule. The term agonist encompasses substances (such as an antibody) which bind to a molecule to promote the activity of that molecule.

An “antagonist” refers to a substance that prevents, blocks, inhibits, neutralizes, or reduces a biological activity or effect of another molecule, such as a receptor. The term antagonist encompasses substances (such as an antibody or antibody conjugate) which bind to a molecule to prevent or reduce the activity of that molecule.

The term “compete”, as used herein with regard to an antibody, means that a first antibody binds to an epitope in a manner sufficiently similar to the binding of a second antibody such that the result of binding of the second antibody with its cognate epitope is detectably decreased in the presence of the first antibody compared to the binding of the second antibody in the absence of the first antibody. The alternative, where the binding of the first antibody to its epitope is also detectably decreased in the presence of the second antibody, can, but need not be the case. That is, a first antibody can inhibit the binding of a second antibody to its epitope without that second antibody inhibiting the binding of the first antibody to its respective epitope. However, where each antibody detectably inhibits the binding of the other antibody with its cognate epitope or ligand, whether to the same, greater, or lesser extent, the antibodies are said to “cross-compete” with each other for binding of their respective epitope(s). Both competing and cross-competing antibodies are encompassed by the present invention. Regardless of the mechanism by which such competition or cross-competition occurs (e.g., steric hindrance, conformational change, or binding to a common epitope, or portion thereof), the skilled artisan would appreciate, based upon the teachings provided herein, that such competing or cross-competing antibodies are encompassed and can be useful for the methods disclosed herein.

Standard competition assays may be used to determine whether two antibodies compete with each other. One suitable assay for antibody competition involves the use of the Biacore technology, which can measure the extent of interactions using surface plasmon resonance (SPR) technology, typically using a biosensor system (such as a BIACORE system). For example, SPR can be used in an in vitro competitive binding inhibition assay to determine the ability of one antibody to inhibit the binding of a second antibody. Another assay for measuring antibody competition uses an ELISA-based approach.

Furthermore, a high throughput process for “binning” antibodies based upon their competition is described in International Patent Application No. WO2003/48731. Competition is present if one antibody (or fragment) reduces the binding of another antibody (or fragment) to an epitope or target of interest. For example, a sequential binding competition assay may be used, with different antibodies being added sequentially. The first antibody may be added to reach binding that is close to saturation. Then, the second antibody is added. If the binding of second antibody to the epitope is not detected, or is significantly reduced (e.g., at least about 10%, at least about 20%, at least about 30%, at least about 40%, at least about 50%, at least about 60%, at least about 70%, at least about 80%, or at least about 90% reduction) as compared to a parallel assay in the absence of the first antibody (which value can be set as 100%), the two antibodies are considered as competing with each other.

An “Fc receptor” (FcR) refers to a receptor that binds to the Fc region of an antibody. In some embodiments, an FcR is a native human FcR. In some embodiments, an FcR is one which binds an IgG antibody (a gamma receptor) and includes receptors of the FcgRI, FcgRII, and FcgRIII subclasses, including allelic variants and alternatively spliced forms of those receptors. FcgRII receptors include FcgRIIA (an “activating receptor”) and FcgRIIB (an “inhibiting receptor”), which have similar amino acid sequences that differ primarily in the cytoplasmic domains thereof. Activating receptor FcgRIIA contains an immunoreceptor tyrosine-based activation motif (ITAM) in its cytoplasmic domain. Inhibiting receptor FcgRIIB contains an immunoreceptor tyrosine-based inhibition motif (ITIM) in its cytoplasmic domain, (see, e.g., Daeron, Annu. Rev. Immunol. 1997; 15:203-234). FcRs are reviewed, for example, in Ravetch and Kinet, Annu. Rev. Immunol 1991; 9:457-92; Capel et al., Immunomethods 1994; 4:25-34; and de Haas et al., J. Lab. Clin. Med. 1995; 126:330-41. Other FcRs, including those to be identified in the future, are encompassed by the term “Fc receptor” herein. The term “Fc receptor” also includes the neonatal receptor, FcRn, which is responsible for the transfer of maternal IgGs to the fetus (Guyer et al., J. Immunol. 1976; 117:587 and Kim et al., J. Immunol. 1994; 24:249) and regulation of homeostasis of immunoglobulins. Methods of measuring binding to FcRn are known (see, e.g., Ghetie and Ward., Immunol. Today 1997; 18(12):592-598; Ghetie et al., Nature Biotechnology, 1997; 15(7):637-640; Hinton et al., J. Biol. Chem. 2004; 279(8):6213-6216; WO 2004/92219).

An “effector cell” refers to a leukocyte which express one or more FcRs and performs effector functions. In certain embodiments, effector cells express at least FcgRIII and perform ADCC effector function(s). Examples of leukocytes which mediate ADCC include peripheral blood mononuclear cells (PBMC), natural killer (NK) cells, monocytes, macrophages, cytotoxic T cells, and neutrophils. Effector cells may be isolated from a native source, e.g., from blood.

The term “antibody-dependent cell-mediated cytotoxicity” or “ADCC” refers to a form of cytotoxicity in which secreted Ig bound onto Fc receptors (FcRs) present on certain cytotoxic cells (e.g., NK cells, neutrophils, and macrophages) enable these cytotoxic effector cells to bind specifically to an antigen-bearing target cell and subsequently kill the target cell with cytotoxins. The primary cells for mediating ADCC, NK cells, express FcgRIII only, whereas monocytes express FcgRI, FcgRII, and FcgRIII. To assess ADCC activity of a molecule of interest, an in vitro ADCC assay, such as that described in U.S. Pat. Nos. 5,500,362, 5,821,337 or 6,737,056, may be performed. Useful effector cells for such assays include PBMC and NK cells. Alternatively, or additionally, ADCC activity of the molecule of interest may be assessed in vivo, e.g., in an animal model such as that disclosed in Clynes et al., Proc. Natl. Acad. Sci. (USA) 1998; 95:652-656. Additional antibodies with altered Fc region amino acid sequences and increased or decreased ADCC activity are described, e.g., in U.S. Pat. Nos. 7,923,538, and 7,994,290.

The term “enhanced ADCC activity” refers to an antibody that is more effective at mediating ADCC in vitro or in vivo compared to the parent antibody, wherein the antibody and the parent antibody differ in at least one structural aspect, and when the amounts of such antibody and parent antibody used in the assay are essentially the same. In some embodiments, the antibody and the parent antibody have the same amino acid sequence, but the antibody is afucosylated while the parent antibody is fucosylated. In some embodiments, ADCC activity will be determined using an in vitro ADCC assay, but other assays or methods for determining ADCC activity, e.g. in an animal model etc., are contemplated. In some embodiments, an antibody with enhanced ADCC activity has enhanced affinity for FcgRIIIA.

The term “altered” FcR binding affinity or ADCC activity refers to an antibody which has either enhanced or diminished activity for one or more of FcR binding activity or ADCC activity compared to a parent antibody, wherein the antibody and the parent antibody differ in at least one structural aspect. An antibody that “displays increased binding” to an FcR binds at least one FcR with better affinity than the parent antibody. An antibody that “displays decreased binding” to an FcR, binds at least one FcR with lower affinity than a parent antibody. Such antibodies that display decreased binding to an FcR may possess little or no appreciable binding to an FcR, e.g., 0-20 percent binding to the FcR compared to a native sequence IgG Fc region.

The term “Complement dependent cytotoxicity” or “CDC” refers to the lysis of a target cell in the presence of complement. Activation of the classical complement pathway is initiated by the binding of the first component of the complement system (C1q) to antibodies (of the appropriate subclass), which are bound to their cognate antigen. To assess complement activation, a CDC assay, e.g., as described in Gazzano-Santoro et al., J. Immunol. Methods 1996; 202: 163, may be performed. Antibodies with altered Fc region amino acid sequences and increased or decreased C1q binding capability are described, e.g., in U.S. Pat. Nos. 6,194,551, 7,923,538, 7,994,290 and WO 1999/51642.

A “host cell” refers to an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this invention.

A “vector” refers to a construct, which is capable of delivering, and, preferably, expressing, one or more gene(s) or sequence(s) of interest (e.g. an antibody-encoding gene) in a host cell. Examples of vectors include, but are not limited to plasmids and viral vectors, and may include naked nucleic acids, or may include nucleic acids associated with delivery-aiding materials (e.g. cationic condensing agents, liposomes, etc.). Vectors may include DNA or RNA. An “expression vector” as used herein refers to a vector that includes at least one polypeptide-encoding gene, at least one regulatory element (e.g. promoter sequence, poly(A) sequence) relating to the transcription or translation of the gene. Typically, a vector used herein contains at least one antibody-encoding gene, as well as one or more of regulatory elements or selectable markers. Vector components may include, for example, one or more of the following: a signal sequence; an origin of replication; one or more marker genes; suitable transcriptional controlling elements (such as promoters, enhancers and terminator). For translation, one or more translational controlling elements may also be included such as ribosome binding sites, translation initiation sites, and stop codons.

An “isolated” molecule (e.g. antibody) refers to a molecule that by virtue of its origin or source of derivation (1) is not associated with naturally associated components that accompany it in its native state, (2) is substantially free of other molecules from the same source, e.g., species, cell from which it is expressed, library, etc., (3) is expressed by a cell from a different species, or (4) does not occur in nature. Thus, a molecule that is chemically synthesized, or expressed in a cellular system different from the system from which it naturally originates, will be “isolated” from its naturally associated components. A molecule also may be rendered substantially free of naturally associated components by isolation, using purification techniques well known in the art. In some embodiments, an isolated antibody is an antibody that does not comprise any peptides disclosed herein.

A “polypeptide” or “protein” (used interchangeably herein) refers to a chain of amino acids of any length. The chain may be linear or branched. The chain may comprise one or more of modified amino acids. The terms also encompass an amino acid chain that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that the polypeptides can occur as single chains or associated chains.

A “polynucleotide” or “nucleic acid,” (used interchangeably herein) refers to a chain of nucleotides of any length, and includes DNA and RNA. The nucleotides can be deoxyribonucleotides, ribonucleotides, modified nucleotides or bases or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside.

A “conservative substitution” refers to replacement of one amino acid by a biologically, chemically or structurally similar residue. Biologically similar means that the substitution does not destroy a biological activity. Structurally similar means that the amino acids have side chains with similar length, such as alanine, glycine and serine or a similar size. Chemical similarity means that the residues have the same charge or are both hydrophilic or hydrophobic. Particular examples include the substitution of a hydrophobic residue, such as isoleucine, valine, leucine or methionine with another, or the substitution of one polar residue for another, such as the substitution of arginine for lysine, glutamic acid for aspartic acid or glutamine for asparagine, serine for threonine, and the like. Particular examples of conservative substitutions include the substitution of a hydrophobic residue such as isoleucine, valine, leucine or methionine for one another, the substitution of a polar residue for another, such as the substitution of arginine for lysine, glutamic acid for aspartic acid, or glutamine for asparagine, and the like. Conservative amino acid substitutions typically include, for example, substitutions within the following groups: glycine, alanine, valine, isoleucine, leucine; aspartic acid, glutamic acid; asparagine, glutamine; serine, threonine; lysine, arginine; and phenylalanine, tyrosine.

The term “identity” or “identical to” refers to the overall relatedness between polymeric molecules, e.g., between nucleic acid molecules (e.g., DNA molecules or RNA molecules) or between polypeptide molecules. “Identity” measures the percent of identical matches between two or more sequences with gap alignments addressed by a particular mathematical model of computer programs (e.g. algorithms), which are well known in the art.

Calculation of the percent identity of two nucleic acid or polypeptide sequences, for example, can be performed by aligning the two sequences for optimal comparison purposes (e.g., gaps can be introduced in one or both of a first and a second sequence for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, considering the number of gaps, and the length of each gap, which need to be introduced for optimal alignment of the two sequences. The comparison of sequences and determination of percent identity between two sequences can be accomplished using a mathematical algorithm.

To determine percent identity, sequences can be aligned using the methods and computer programs, including BLAST, available over the world wide web at ncbi.nlm.nih.gov/BLAST/. Other alignment programs include MegAlign® program in the Lasergene® suite of bioinformatics software (DNASTAR®, Inc., Madison, WI). Another alignment algorithm is FASTA, available in the Genetics Computing Group (GCG) package, from Madison, Wis., USA. Other techniques for alignment are described in in ABELSON, J. N., et al. (eds.), Computer Methods for Macromolecular Sequence Analysis (Volume 266), Methods in Enzymology. 1^{st} Ed. Academic Press, Inc., 1996. Of particular interest are alignment programs that permit gaps in the sequence. Smith-Waterman is one type of algorithm that permits gaps in sequence alignments. See SHPAER, E. G., “GeneAssist. Smith-Waterman and other database similarity searches and identification of motifs,” Methods in Molecular Biology, 1997, 70:173-187. Also, the GAP program using the Needleman and Wunsch alignment method can be utilized to align sequences. See NEEDLEMAN, S. B. and WUNSCH, C. D., “A General Method Applicable to the Search for Similarities in the Amino Acid Sequences of Two Proteins,” Journal of Molecular Biology, 1970, 48:443-453.

Also, of interest is the BestFit program using the local homology algorithm of Smith and Waterman (SMITH, T. S. and WATERMAN, M. S., “Comparison of biosequences,” Advances in Applied Mathematics, 1981, 2(4):482) to determine sequence identity. The gap generation penalty will generally range from 1 to 5, usually 2 to 4 and in some embodiments will be 3. The gap extension penalty will generally range from about 0.01 to 0.20 and in some instances will be 0.10. The program has default parameters determined by the sequences inputted to be compared. Preferably, the sequence identity is determined using the default parameters determined by the program. This program is available also from GCG package, from Madison, WI, USA.

Another program of interest is the FastDB algorithm. FastDB is described in SCHLESINGER, D. H., “Current Methods in Sequence Comparison and Analysis.” Macromolecule Sequencing and Synthesis, Selected Methods and Applications, edited by David H. Schlesinger, Alan R. Liss, Inc., 1988, 127-149 Percent sequence identity is calculated by FastDB based upon the following parameters: Mismatch Penalty: 1.00; Gap Penalty: 1.00; Gap Size Penalty: 0.33; and Joining Penalty: 30.0.

The terms “increase,” improve,” “decrease” or “reduce” refer to values that are relative to a baseline measurement, such as a measurement in the same individual prior to initiation of treatment described herein, or a measurement in a control individual or subject (or multiple control individuals or subjects) in the absence of the treatment described herein. In some embodiments, a “control individual” is an individual afflicted with the same form of disease or injury as an individual being treated. In some embodiments, a “control individual” is an individual that is not afflicted with the same form of disease or injury as an individual being treated.

The term ‘excipient’ refers to any material which, which combined with an active ingredient of interest (e.g. antibody), allow the active ingredient to retain biological activity. The choice of excipient will to a large extent depend on factors such as the mode of administration, the effect of the excipient on solubility and stability, and the nature of the dosage form. As used herein, “excipient”” includes any and all solvents, dispersion media, coatings, antibacterial and antifungal agents, isotonic and absorption delaying agents, carriers, diluents and the like that are physiologically compatible. Examples of an excipient include one or more of water, saline, phosphate buffered saline, dextrose, glycerol, ethanol and the like, as well as combinations thereof, and may include isotonic agents, for example, sugars, sodium chloride, or polyalcohols such as mannitol, or sorbitol in the composition.

The terms “treating”, “treat” or “treatment” refer to any type of treatment, e.g. such as to relieve, alleviate, or slow the progression of the patient's disease, disorder or condition or any tissue damage associated with the disease. In some embodiments, the disease, disorder or condition is a metabolic disease, such as obesity or diabetes.

The terms “prevent” or “prevention” refer to one or more of delay of onset, reduction in frequency, or reduction in severity of at least one sign or symptom. In some embodiments, prevention is assessed on a population basis such that an agent is considered to “prevent” a particular disease, disorder or condition if a statistically significant decrease in the development, frequency or intensity of one or more symptoms of the disease, disorder or condition is observed in a population susceptible to the disease, disorder or condition. Prevention may be considered complete when onset of disease, disorder or condition has been delayed for a predefined period of time.

The terms “subject, “individual” or “patient,” (used interchangeably herein), refer to any animal, including mammals. Mammals according to the invention include canine, feline, bovine, caprine, equine, ovine, porcine, rodents, lagomorphs, primates, humans and the like, and encompass mammals in utero. In an embodiment, humans are suitable subjects. Human subjects may be of any gender and at any stage of development.

The term “therapeutically effective amount” refers to the amount of active ingredient that elicits the biological or medicinal response in a tissue, system, animal, individual or human that is being sought by a researcher, veterinarian, medical doctor or other clinician, which may include one or more of the following:
(1) preventing the disease; for example, preventing a disease, condition or disorder in an individual that may be predisposed to the disease, condition or disorder but does not yet experience or display the pathology or symptomatology of the disease;
(2) inhibiting the disease; for example, inhibiting a disease, condition or disorder in an individual that is experiencing or displaying the pathology or symptomatology of the disease, condition or disorder (i.e., arresting or slowing further development of the pathology or symptomatology); and
(3) ameliorating the disease; for example, ameliorating a disease, condition or disorder in an individual that is experiencing or displaying the pathology or symptomatology of the disease, condition or disorder (i.e., reversing the pathology or symptomatology).

### Anti-GIPR Antibodies

GIPR belongs to the glucagon subfamily of class 1 G protein-coupled receptors (GPCRs) characterized by an extracellular N-terminal domain, seven transmembrane domains and an intracellular C-terminus (See e.g. -Zhao et al. Nat Commun. 2022, 13:1057). The N-terminal extracellular domain forms the primary peptide recognition and binding site of the receptor.

As used herein, the term GIPR, includes variants, isoforms, homologs, orthologs and paralogs of GIPR. In some embodiments, an antibody disclosed herein cross-reacts with GIPR, respectively, from species other than human, such as GIPR of cynomolgus monkey, as well as different forms of GIPR, respectively. In some embodiments, an antibody may be completely specific for human GIPR and may not exhibit species cross-reactivity (e.g., does not bind mouse GIPR) or other types of cross-reactivity. As used herein the term GIPR refers to naturally occurring human GIPR, respectively, unless contextually dictated otherwise. Therefore, an “GIPR antibody”, “anti-GIPR antibody”, or other similar designation means any antibody (as defined herein) that binds or reacts with GIPR, an isoform, fragment or derivative thereof. Without wishing to be bound by any particular theory, blockade of GIP and GIPR interaction inhibits GIPR signaling.

A neutralizing or “blocking” antibody or bispecific antibody refers to an antibody or bispecific antibody whose binding to GIPR, (i) interferes with, limits, or inhibits the interaction between GIPR and a GIPR ligand or (ii) results in inhibition of at least one biological function of GIPR signaling. Assays to determine neutralization by an antibody of the disclosure are well-known in the art.

“Biological function” or “biological activity” of GIPR, respectively, is meant to include the binding between GIPR and its ligand. The biological function or biological activity of GIPR can, but need not be, mediated by the interaction between GIPR and its ligands.

Table 1 shows sequences of novel anti-GIPR antibodies.

TABLE 1
Amino Acid and Nucleotide Sequences of anti-GIPR Antibodies

| | | SEQ |
|---|---|---|
| Peptide/ | | ID |
| Nucleotide | Sequence | NO. |
| GIPR-0107 | QVQLVQSGAE VKKPGSSVKV SCKASGYTFS | 1 |
| Heavy chain | SYAINWVRQA PGQGLEWMGG IVPIFGIANY | |
| variable | AQKFQGRVTI TADESTSTAY MELSSLRSED | |
| region | TAVYYCARGS DYVVPHFDYW GQGTLVTVSS | |
| (VH) | | |
| GIPR-0107 | GYTFSSY | 2 |
| CDR-H1 | | |
| (Chothia) | | |
| GIPR-0107 | | 3 |
| CDR-H1 | SYAIN | |
| (Kabat) | | |
| GIPR-0107 | GYTFSSYAIN | 4 |
| CDR-H1 | | |
| (extended) | | |
| GIPR-0107 | GIVPIFGI | 5 |
| CDR-H2 | | |
| (Chothia) | | |
| GIPR-0107 | GIVPIFGIANYAQKFQG | 6 |
| CDR-H2 | | |
| (Kabat) | | |
| GIPR-0107 | GSDYVVPHFDY | 7 |
| CDR-H3 | | |
| GIPR-0107 | QVQLVQSGAE VKKPGSSVKV SCKASGYTFS | 8 |
| Heavy chain | SYAINWVRQA PGQGLEWMGG IVPIFGIANY | |
| | AQKFQGRVTI TADESTSTAY MELSSLRSED | |
| | TAVYYCARGS DYVVPHFDYW GQGTLVTVSS | |
| | ASTKGPSVFP LAPSSKSTSG GTAALGCLVK | |
| | DYFPEPVTVS WNSGALTSGV HTFPAVLQSS | |
| | GLYSLSSVVT VPSSSLGTQT YICNVNHKPS | |
| | NTKVDKKVEP KSCDKTHTCP PCPAPEAAGA | |
| | PSVFLFPPKP KDTLMISRTP EVTCVVVDVS | |
| | HEDPEVKFNW YVDGVEVHNA KTKPREEQYN | |
| | STYRVVSVLT VLHQDWLNGK EYKCKVSNKA | |
| | LPAPIEKTIS KAKGQPREPQ VYTLPPSREE | |
| | MTKNQVSLTC LVKGFYPSDI AVEWESNGQP | |
| | ENNYKTTPPV LDSDGSFFLY SKLTVDKSRW | |
| | QQGNVFSCSV MHEALHNHYT QKSLSLSPGK | |
| GIPR-0107 | QSVLTQPPSA SGTPGQRVTI SCSGSSSNIG | 9 |
| Light chain | SNYVYWYQQL PGTAPKLLIY RNNQRPSGVP | |
| variable region | DRFSGSKSGT SASLAISGLR SEDEADYYCA | |
| (VL) | AWDDSLSGVV FGGGTKLTVL | |
| GIPR-0107 | SGSSSNIGSNYVY | 10 |
| CDR-L1 | | |
| GIPR-0107 | RNNQRPS | 11 |
| CDR-L2 | | |
| GIPR-0107 | AAWDDSLSGVV | 12 |
| CDR-L3 | | |
| GIPR-0107 | QSVLTQPPSA SGTPGQRVTI SCSGSSSNIG | 13 |
| Light chain | SNYVYWYQQL PGTAPKLLIY RNNQRPSGVP | |
| (Lambda) | DRFSGSKSGT SASLAISGLR SEDEADYYCA | |
| | AWDDSLSGVV FGGGTKLTVL GQPKAAPSVT | |
| | LFPPSSEELQ ANKATLVCLI SDFYPGAVTV | |
| | AWKADSSPVK AGVETTTPSK QSNNKYAASS | |
| | YLSLTPEQWK SHRSYSCQVT HEGSTVEKTV | |
| | APTECS | |
| GIPR-2767 | EVQLVESGGG LVKPGGSLRL SCAASGFPFS | 14 |
| Heavy chain | SAWMSWVRQA PGKGLEWVGR IKSKADAGTT | |
| variable region | DYGAPVKGRF TISRDDSKNT LYLQMNSLKT | |
| (VH) | EDTAVYYCTT EDIVGVPFKG YYQYMDAWGK | |
| | GTTVTVSS | |
| GIPR-2767 | GFPFSSA | 15 |
| CDR-H1 | | |
| (Chothia) | | |
| GIPR-2767 | SAWMS | 16 |
| CDR-H1 | | |
| (Kabat) | | |
| GIPR-2767 | GFPFSSAWMS | 17 |
| CDR-H1 | | |
| (Extended) | | |
| GIPR-2767 | RIKSKADAGT | 18 |
| CDR-H2 | | |
| (Chothia) | | |
| GIPR-2767 | RIKSKADAGTTDYGAPVKG | 19 |
| CDR-H2 | | |
| (Kabat) | | |
| GIPR-2767 | EDIVGVPFKGYYQYMDA | 20 |
| CDR-H3 | | |
| GIPR-2767 | EVQLVESGGG LVKPGGSLRL SCAASGFPFS | 21 |
| Heavy chain | SAWMSWVRQA PGKGLEWVGR IKSKADAGTT | |
| | DYGAPVKGRF TISRDDSKNT LYLQMNSLKT | |
| | EDTAVYYCTT EDIVGVPFKG YYQYMDAWGK | |
| | GTTVTVSSAS TKGPSVFPLA PSSKSTSGGT | |
| | AALGCLVKDY FPEPVTVSWN SGALTSGVHT | |
| | FPAVLQSSGL YSLSSVVTVP SSSLGTQTYI | |
| | CNVNHKPSNT KVDKKVEPKS CDKTHTCPPC | |
| | PAPEAAGAPS VFLFPPKPKD TLMISRTPEV | |
| | TCVVVDVSHE DPEVKFNWYV DGVEVHNAKT | |
| | KPREEQYNST YRVVSVLTVL HQDWLNGKEY | |
| | KCKVSNKALP APIEKTISKA KGQPREPQVY | |
| | TLPPSREEMT KNQVSLTCLV KGFYPSDIAV | |
| | EWESNGQPEN NYKTTPPVLD SDGSFFLYSK | |
| | LTVDKSRWQQ GNVFSCSVMH EALHNHYTQK | |
| | SLSLSPGK | |
| GIPR-2767 | DIQMTQSPSS LSASVGDRVT ITCRASQSIS | 22 |
| Light chain | SYLNWYQQKP GKAPKLLIYA ASSLQSGVPS | |
| variable region | RFSGSGSGTD FTLTISSLQP EDFATYYCQQ | |
| (VL) | SYSTPLTFGQ GTKVEIK | |
| GIPR-2767 | RASQSISSYLN | 23 |
| CDR-L1 | | |
| GIPR-2767 | AASSLQS | 24 |
| CDR-L2 | | |
| GIPR-2767 | QQSYSTPLT | 25 |
| CDR-L3 | | |
| GIPR-2767 | DIQMTQSPSS LSASVGDRVT ITCRASQSIS | 26 |
| Light chain | SYLNWYQQKP GKAPKLLIYA ASSLQSGVPS | |
| (Kappa) | RFSGSGSGTD FTLTISSLQP EDFATYYCQQ | |
| | SYSTPLTFGQ GTKVEIKRTV AAPSVFIFPP | |
| | SDEQLKSGTA SVVCLLNNFY PREAKVQWKV | |
| | DNALQSGNSQ ESVTEQDSKD STYSLSSTLT | |
| | LSKADYEKHK VYACEVTHQG LSSPVTKSEN | |
| | RGEC | |
| GIPR-0107 | QVQLVQSGAE VKKPGSSVKV SCKASGYTFS | 27 |
| heavy chain | SYAINWVRQA PGQGLEWMGG IVPIFGIANY | |
| (HC) K290C | AQKFQGRVTI TADESTSTAY MELSSLRSED | |
| | TAVYYCARGS DYVVPHFDYW GQGTLVTVSS | |
| | ASTKGPSVFP LAPSSKSTSG GTAALGCLVK | |
| | DYFPEPVTVS WNSGALTSGV HTFPAVLQSS | |
| | GLYSLSSVVT VPSSSLGTQT YICNVNHKPS | |
| | NTKVDKKVEP KSCDKTHTCP PCPAPEAAGA | |
| | PSVFLFPPKP KDTLMISRTP EVTCVVVDVS | |
| | HEDPEVKFNW YVDGVEVHNA KTCPREEQYN | |
| | STYRVVSVLT VLHQDWLNGK EYKCKVSNKA | |
| | LPAPIEKTIS KAKGQPREPQ VYTLPPSREE | |
| | MTKNQVSLTC LVKGFYPSDI AVEWESNGQP | |
| | ENNYKTTPPV LDSDGSFFLY SKLTVDKSRW | |
| | QQGNVFSCSV MHEALHNHYT QKSLSLSPGK | |
| GIPR-0107 | QVQLVQSGAE VKKPGSSVKV SCKASGYTFS | 28 |
| heavy chain | SYAINWVRQA PGQGLEWMGG IVPIFGIANY | |
| (HC) E272C | AQKFQGRVTI TADESTSTAY MELSSLRSED | |
| | TAVYYCARGS DYVVPHFDYW GQGTLVTVSS | |
| | ASTKGPSVFP LAPSSKSTSG GTAALGCLVK | |
| | DYFPEPVTVS WNSGALTSGV HTFPAVLQSS | |
| | GLYSLSSVVT VPSSSLGTQT YICNVNHKPS | |
| | NTKVDKKVEP KSCDKTHTCP PCPAPEAAGA | |
| | PSVFLFPPKP KDTLMISRTP EVTCVVVDVS | |
| | HEDPCVKFNW YVDGVEVHNA KTKPREEQYN | |
| | STYRVVSVLT VLHQDWLNGK EYKCKVSNKA | |
| | LPAPIEKTIS KAKGQPREPQ VYTLPPSREE | |
| | MTKNQVSLTC LVKGFYPSDI AVEWESNGQP | |
| | ENNYKTTPPV LDSDGSFFLY SKLTVDKSRW | |
| | QQGNVFSCSV MHEALHNHYT QKSLSLSPGK | |
| GIPR-0107 light | QSVLTQPPSA SGTPGQRVTI SCSGSSSNIG | 29 |
| chain (Lambda) | SNYVYWYQQL PGTAPKLLIY RNNQRPSGVP | |
| (LC) V205C | DRFSGSKSGT SASLAISGLR SEDEADYYCA | |
| | AWDDSLSGVV FGGGTKLTVL GQPKAAPSVT | |
| | LFPPSSEELQ ANKATLVCLI SDFYPGAVTV | |
| | AWKADSSPVK AGVETTTPSK QSNNKYAASS | |
| | YLSLTPEQWK SHRSYSCQVT HEGSTCEKTV | |
| | APTECS | |
| GIPR-2767 | EVQLVESGGG LVKPGGSLRL SCAASGFPFS | 30 |
| heavy chain | SAWMSWVRQA PGKGLEWVGR IKSKADAGTT | |
| (HC) K290C | DYGAPVKGRF TISRDDSKNT LYLQMNSLKT | |
| | EDTAVYYCTT EDIVGVPFKG YYQYMDAWGK | |
| | GTTVTVSSAS TKGPSVFPLA PSSKSTSGGT | |
| | AALGCLVKDY FPEPVTVSWN SGALTSGVHT | |
| | FPAVLQSSGL YSLSSVVTVP SSSLGTQTYI | |
| | CNVNHKPSNT KVDKKVEPKS CDKTHTCPPC | |
| | PAPEAAGAPS VFLFPPKPKD TLMISRTPEV | |
| | TCVVVDVSHE DPEVKFNWYV DGVEVHNAKT | |
| | CPREEQYNST YRVVSVLTVL HQDWLNGKEY | |
| | KCKVSNKALP APIEKTISKA KGQPREPQVY | |
| | TLPPSREEMT KNQVSLTCLV KGFYPSDIAV | |
| | EWESNGQPEN NYKTTPPVLD SDGSFFLYSK | |
| | LTVDKSRWQQ GNVFSCSVMH EALHNHYTQK | |
| | SLSLSPGK | |
| GIPR-2767 light | DIQMTQSPSS LSASVGDRVT ITCRASQSIS | 31 |
| chain | SYLNWYQQKP GKAPKLLIYA ASSLQSGVPS | |
| (Kappa) | RFSGSGSGTD FTLTISSLQP EDFATYYCQQ | |
| (LC) K183C | SYSTPLTFGQ GTKVEIKRTV AAPSVFIFPP | |
| | SDEQLKSGTA SVVCLLNNFY PREAKVQWKV | |
| | DNALQSGNSQ ESVTEQDSKD STYSLSSTLT | |
| | LSCADYEKHK VYACEVTHQG LSSPVTKSEN | |
| | RGEC | |
| GIPR-2767 | EVQLVESGGG LVKPGGSLRL SCAASGFPFS | 32 |
| heavy chain | SAWMSWVRQA PGKGLEWVGR IKSKADAGTT | |
| (HC) E272C | DYGAPVKGRF TISRDDSKNT LYLQMNSLKT | |
| | EDTAVYYCTT EDIVGVPFKG YYQYMDAWGK | |
| | GTTVTVSSAS TKGPSVFPLA PSSKSTSGGT | |
| | AALGCLVKDY FPEPVTVSWN SGALTSGVHT | |
| | FPAVLQSSGL YSLSSVVTVP SSSLGTQTYI | |
| | CNVNHKPSNT KVDKKVEPKS CDKTHTCPPC | |
| | PAPEAAGAPS VFLFPPKPKD TLMISRTPEV | |
| | TCVVVDVSHE DPCVKFNWYV DGVEVHNAKT | |
| | KPREEQYNST YRVVSVLTVL HQDWLNGKEY | |
| | KCKVSNKALP APIEKTISKA KGQPREPQVY | |
| | TLPPSREEMT KNQVSLTCLV KGFYPSDIAV | |
| | EWESNGQPEN NYKTTPPVLD SDGSFFLYSK | |
| | LTVDKSRWQQ GNVFSCSVMH EALHNHYTQK | |
| | SLSLSPGK | |
| GIPR extra | RAETGSKGQT AGELYQRWER YRRECQETLA | 33 |
| cellular domain | AAEPPSGLAC NGSFDMYVCW DYAAPNATAR | |
| (ECD) | ASCPWYLPWH HHVAAGFVLR QCGSDGQWGL | |
| | WRDHTQCENP EKNEAFLDQR LILERLQLEV | |
| | LFQGPGLNDI FEAQKIEWHE GSENLYFQ | |
| Full length | MTTSPILQLL LRLSLCGLLL QRAETGSKGQ | 148 |
| GIPR | TAGELYQRWE RYRRECQETL AAAEPPSGLA | |
| | CNGSFDMYVC WDYAAPNATA RASCPWYLPW | |
| | HHHVAAGFVL RQCGSDGQWG LWRDHTQCEN | |
| | PEKNEAFLDQ RLILERLQVM YTVGYSLSLA | |
| | TLLLALLILS LFRRLHCTRN YIHINLFTSF | |
| | MLRAAAILSR DRLLPRPGPY LGDQALALWN | |
| | QALAACRTAQ IVTQYCVGAN YTWLLVEGVY | |
| | LHSLLVLVGG SEEGHFRYYL LLGWGAPALF | |
| | VIPWVIVRYL YENTQCWERN EVKAIWWIIR | |
| | TPILMTILIN FLIFIRILGI LLSKLRTRQM | |
| | RCRDYRLRLA RSTLTLVPLL GVHEVVFAPV | |
| | TEEQARGALR FAKLGFEIFL SSFQGFLVSV | |
| | LYCFINKEVQ SEIRRGWHHC RLRRSLGEEQ | |
| | RQLPERAFRA LPSGSGPGEV PTSRGLSSGT | |
| | LPGPGNEASR ELESYC | |
| GIPR-0107 | QVQLVQSGAE VKKPGSSVKV SCKASGYTFS | 34 |
| Heavy chain | SYAINWVRQA PGQGLEWMGG IVPIFGIANY | |
| A118C | AQKFQGRVTI TADESTSTAY MELSSLRSED | |
| | TAVYYCARGS DYVVPHFDYW GQGTLVTVSS | |
| | CSTKGPSVFP LAPSSKSTSG GTAALGCLVK | |
| | DYFPEPVTVS WNSGALTSGV HTFPAVLQSS | |
| | GLYSLSSVVT VPSSSLGTQT YICNVNHKPS | |
| | NTKVDKKVEP KSCDKTHTCP PCPAPEAAGA | |
| | PSVFLFPPKP KDTLMISRTP EVTCVVVDVS | |
| | HEDPEVKFNW YVDGVEVHNA KTKPREEQYN | |
| | STYRVVSVLT VLHQDWLNGK EYKCKVSNKA | |
| | LPAPIEKTIS KAKGQPREPQ VYTLPPSREE | |
| | MTKNQVSLTC LVKGFYPSDI AVEWESNGQP | |
| | ENNYKTTPPV LDSDGSFFLY SKLTVDKSRW | |
| | QQGNVFSCSV MHEALHNHYT QKSLSLSPGK | |
| GIPR-0107 | QVQLVQSGAE VKKPGSSVKV SCKASGYTFS | 35 |
| Heavy chain | SYAINWVRQA PGQGLEWMGG IVPIFGIANY | |
| E380C | AQKFQGRVTI TADESTSTAY MELSSLRSED | |
| | TAVYYCARGS DYVVPHFDYW GQGTLVTVSS | |
| | ASTKGPSVFP LAPSSKSTSG GTAALGCLVK | |
| | DYFPEPVTVS WNSGALTSGV HTFPAVLQSS | |
| | GLYSLSSVVT VPSSSLGTQT YICNVNHKPS | |
| | NTKVDKKVEP KSCDKTHTCP PCPAPEAAGA | |
| | PSVFLFPPKP KDTLMISRTP EVTCVVVDVS | |
| | HEDPEVKFNW YVDGVEVHNA KTKPREEQYN | |
| | STYRVVSVLT VLHQDWLNGK EYKCKVSNKA | |
| | LPAPIEKTIS KAKGQPREPQ VYTLPPSREE | |
| | MTKNQVSLTC LVKGFYPSDI AVCWESNGQP | |
| | ENNYKTTPPV LDSDGSFFLY SKLTVDKSRW | |
| | QQGNVFSCSV MHEALHNHYT QKSLSLSPGK | |
| GIPR-0107 | QVQLVQSGAE VKKPGSSVKV SCKASGYTFS | 36 |
| Heavy chain | SYAINWVRQA PGQGLEWMGG IVPIFGIANY | |
| L443C | AQKFQGRVTI TADESTSTAY MELSSLRSED | |
| | TAVYYCARGS DYVVPHFDYW GQGTLVTVSS | |
| | ASTKGPSVFP LAPSSKSTSG GTAALGCLVK | |
| | DYFPEPVTVS WNSGALTSGV HTFPAVLQSS | |
| | GLYSLSSVVT VPSSSLGTQT YICNVNHKPS | |
| | NTKVDKKVEP KSCDKTHTCP PCPAPEAAGA | |
| | PSVFLFPPKP KDTLMISRTP EVTCVVVDVS | |
| | HEDPEVKFNW YVDGVEVHNA KTKPREEQYN | |
| | STYRVVSVLT VLHQDWLNGK EYKCKVSNKA | |
| | LPAPIEKTIS KAKGQPREPQ VYTLPPSREE | |
| | MTKNQVSLTC LVKGFYPSDI AVEWESNGQP | |
| | ENNYKTTPPV LDSDGSFFLY SKLTVDKSRW | |
| | QQGNVFSCSV MHEALHNHYT QKSLSCSPGK | |
| GIPR-2767 | EVQLVESGGG LVKPGGSLRL SCAASGFPFS | 37 |
| heavy chain | SAWMSWVRQA PGKGLEWVGR IKSKADAGTT | |
| (HC) A118C | DYGAPVKGRF TISRDDSKNT LYLQMNSLKT | |
| | EDTAVYYCTT EDIVGVPFKG YYQYMDAWGK | |
| | GTTVTVSSCS TKGPSVFPLA PSSKSTSGGT | |
| | AALGCLVKDY FPEPVTVSWN SGALTSGVHT | |
| | FPAVLQSSGL YSLSSVVTVP SSSLGTQTYI | |
| | CNVNHKPSNT KVDKKVEPKS CDKTHTCPPC | |
| | PAPEAAGAPS VFLFPPKPKD TLMISRTPEV | |
| | TCVVVDVSHE DPEVKFNWYV DGVEVHNAKT | |
| | KPREEQYNST YRVVSVLTVL HQDWLNGKEY | |
| | KCKVSNKALP APIEKTISKA KGQPREPQVY | |
| | TLPPSREEMT KNQVSLTCLV KGFYPSDIAV | |
| | EWESNGQPEN NYKTTPPVLD SDGSFFLYSK | |
| | LTVDKSRWQQ GNVFSCSVMH EALHNHYTQK | |
| | SLSLSPGK | |
| GIPR-2767 | EVQLVESGGG LVKPGGSLRL SCAASGFPFS | 38 |
| heavy chain | SAWMSWVRQA PGKGLEWVGR IKSKADAGTT | |
| (HC) E380C | DYGAPVKGRF TISRDDSKNT LYLQMNSLKT | |
| | EDTAVYYCTT EDIVGVPFKG YYQYMDAWGK | |
| | GTTVTVSSAS TKGPSVFPLA PSSKSTSGGT | |
| | AALGCLVKDY FPEPVTVSWN SGALTSGVHT | |
| | FPAVLQSSGL YSLSSVVTVP SSSLGTQTYI | |
| | CNVNHKPSNT KVDKKVEPKS CDKTHTCPPC | |
| | PAPEAAGAPS VFLFPPKPKD TLMISRTPEV | |
| | TCVVVDVSHE DPEVKENWYV DGVEVHNAKT | |
| | KPREEQYNST YRVVSVLTVL HQDWLNGKEY | |
| | KCKVSNKALP APIEKTISKA KGQPREPQVY | |
| | TLPPSREEMT KNQVSLTCLV KGFYPSDIAV | |
| | CWESNGQPEN NYKTTPPVLD SDGSFFLYSK | |
| | LTVDKSRWQQ GNVFSCSVMH EALHNHYTQK | |
| | SLSLSPGK | |
| GIPR-2767 | EVQLVESGGG LVKPGGSLRL SCAASGFPFS | 39 |
| heavy chain | SAWMSWVRQA PGKGLEWVGR IKSKADAGTT | |
| (HC) L443C | DYGAPVKGRF TISRDDSKNT LYLQMNSLKT | |
| | EDTAVYYCTT EDIVGVPFKG YYQYMDAWGK | |
| | GTTVTVSSAS TKGPSVFPLA PSSKSTSGGT | |
| | AALGCLVKDY FPEPVTVSWN SGALTSGVHT | |
| | FPAVLQSSGL YSLSSVVTVP SSSLGTQTYI | |
| | CNVNHKPSNT KVDKKVEPKS CDKTHTCPPC | |
| | PAPEAAGAPS VFLFPPKPKD TLMISRTPEV | |
| | TCVVVDVSHE DPEVKFNWYV DGVEVHNAKT | |
| | KPREEQYNST YRVVSVLTVL HQDWLNGKEY | |
| | KCKVSNKALP APIEKTISKA KGQPREPQVY | |
| | TLPPSREEMT KNQVSLTCLV KGFYPSDIAV | |
| | EWESNGQPEN NYKTTPPVLD SDGSFFLYSK | |
| | LTVDKSRWQQ GNVFSCSVMH EALHNHYTQK | |
| | SLSCSPGK | |
| GIPR-2767 | GAGGTGCAGCTCGTGGAAAGCGGAGGAGGCCTG | 155 |
| VH DNA | GTGAAACCTGGCGGCTCCCTGCGGCTGAGCTGC | |
| | GCCGCTTCTGGCTTCCCCTTCAGCAGCGCCTGG | |
| | ATGAGCTGGGTCAGACAGGCCCCAGGCAAAGGC | |
| | CTGGAATGGGTGGGCCGGATCAAGTCTAAGGCC | |
| | GACGCCGGCACAACAGACTACGGCGCCCCTGTG | |
| | AAGGGAAGATTCACCATCAGCAGAGATGACTCT | |
| | AAGAACACCCTGTACCTGCAAATGAACAGCCTG | |
| | AAGACCGAGGACACCGCCGTGTACTATTGTACC | |
| | ACAGAGGATATCGTGGGCGTGCCTTTTAAGGGC | |
| | TACTACCAGTACATGGACGCTTGGGGCAAGGGC | |
| | ACCACCGTGACAGTGTCCAGC | |
| GIPR-2767 | GAGGTGCAGCTCGTGGAAAGCGGAGGAGGCCTG | 156 |
| HC DNA | GTGAAACCTGGCGGCTCCCTGCGGCTGAGCTGC | |
| | GCCGCTTCTGGCTTCCCCTTCAGCAGCGCCTGG | |
| | ATGAGCTGGGTCAGACAGGCCCCAGGCAAAGGC | |
| | CTGGAATGGGTGGGCCGGATCAAGTCTAAGGCC | |
| | GACGCCGGCACAACAGACTACGGCGCCCCTGTG | |
| | AAGGGAAGATTCACCATCAGCAGAGATGACTCT | |
| | AAGAACACCCTGTACCTGCAAATGAACAGCCTG | |
| | AAGACCGAGGACACCGCCGTGTACTATTGTACC | |
| | ACAGAGGATATCGTGGGCGTGCCTTTTAAGGGC | |
| | TACTACCAGTACATGGACGCTTGGGGCAAGGGC | |
| | ACCACCGTGACAGTGTCCAGCGCGTCGACCAAG | |
| | GGCCCATCGGTCTTCCCCCTGGCACCCTCCTCC | |
| | AAGAGCACCTCTGGGGGCACAGCGGCCCTGGGC | |
| | TGCCTGGTCAAGGACTACTTCCCCGAACCGGTG | |
| | ACGGTGTCGTGGAACTCAGGCGCCCTGACCAGC | |
| | GGCGTGCACACCTTCCCGGCTGTCCTACAGTC | |
| | CTCAGGACTCTACTCCCTCAGCAGCGTGGTGAC | |
| | CGTGCCCTCCAGCAGCTTGGGCACCCAGACCTA | |
| | CATCTGCAACGTGAATCACAAGCCCAGCAACAC | |
| | CAAGGTGGACAAGAAAGTTGAGCCCAAATCTTG | |
| | TGACAAAACTCACACATGCCCACCGTGCCCAGC | |
| | ACCTGAAGCCGCTGGGGCACCGTCAGTCTTCCT | |
| | CTTCCCCCCAAAACCCAAGGACACCCTCATGAT | |
| | CTCCCGGACCCCTGAGGTCACATGCGTGGTGGT | |
| | GGACGTGAGCCACGAAGACCCTGAGGTCAAGTT | |
| | CAACTGGTACGTGGACGGCGTGGAGGTGCATAA | |
| | TGCCAAGACAAAGCCGCGGGAGGAGCAGTACAA | |
| | CAGCACGTACCGTGTGGTCAGCGTCCTCACCGT | |
| | CCTGCACCAGGACTGGCTGAATGGCAAGGAGTA | |
| | CAAGTGCAAGGTCTCCAACAAAGCCCTCCCAGC | |
| | CCCCATCGAGAAAACCATCTCCAAAGCCAAAGG | |
| | GCAGCCCCGAGAACCACAGGTGTACACCCTGCC | |
| | CCCATCCCGGGAGGAGATGACCAAGAACCAGGT | |
| | CAGCCTGACCTGCCTGGTCAAAGGCTTCTATCC | |
| | CAGCGACATCGCCGTGGAGTGGGAGAGCAATGG | |
| | GCAGCCGGAGAACAACTACAAGACCACGCCTCC | |
| | CGTGCTGGACTCCGACGGCTCCTTCTTCCTCTA | |
| | TAGCAAGCTCACCGTGGACAAGAGCAGGTGGCA | |
| | GCAGGGGAACGTCTTCTCATGCTCCGTGATGCA | |
| | TGAGGCTCTGCACAACCACTACACGCAGAAGAG | |
| | CCTCTCCCTGTCCCCGGGAAAA | |
| GIPR-2767 VL | GACATCCAGATGACCCAGAGCCCCAGCAGCCTG | 157 |
| DNA | TCTGCCAGCGTGGGCGACAGAGTGACCATCACC | |
| | TGTAGAGCCAGCCAGAGCATCAGCAGCTACCTG | |
| | AACTGGTATCAGCAGAAGCCCGGCAAGGCCCCC | |
| | AAACTGCTGATCTACGCCGCCAGCTCTCTGCAG | |
| | TCTGGCGTGCCCAGCAGATTTTCCGGCAGCGGC | |
| | TCTGGCACCGACTTCACCCTGACCATCAGCTCC | |
| | CTGCAGCCCGAGGACTTCGCCACCTACTACTGC | |
| | CAGCAGAGCTACAGCACCCCCCTGACCTTTGGC | |
| | CAGGGCACCAAGGTGGAAATCAAA | |
| GIPR-2767 LC | GACATCCAGATGACCCAGAGCCCCAGCAGCCTG | 158 |
| DNA | TCTGCCAGCGTGGGCGACAGAGTGACCATCACC | |
| | TGTAGAGCCAGCCAGAGCATCAGCAGCTACCTG | |
| | AACTGGTATCAGCAGAAGCCCGGCAAGGCCCCC | |
| | AAACTGCTGATCTACGCCGCCAGCTCTCTGCAG | |
| | TCTGGCGTGCCCAGCAGATTTTCCGGCAGCGGC | |
| | TCTGGCACCGACTTCACCCTGACCATCAGCTCC | |
| | CTGCAGCCCGAGGACTTCGCCACCTACTACTGC | |
| | CAGCAGAGCTACAGCACCCCCCTGACCTTTGGC | |
| | CAGGGCACCAAGGTGGAAATCAAACGTACGGTG | |
| | GCTGCACCATCTGTCTTCATCTTCCCGCCATCT | |
| | GATGAGCAGTTGAAATCTGGAACTGCCTCTGTT | |
| | GTGTGCCTGCTGAATAACTTCTATCCCAGAGAG | |
| | GCCAAAGTACAGTGGAAGGTGGATAACGCCCTC | |
| | CAATCGGGTAACTCCCAGGAGAGTGTCACAGAG | |
| | CAGGACAGCAAGGACAGCACCTACAGCCTCAGC | |
| | AGCACCCTGACGCTGAGCAAAGCAGACTACGAG | |
| | AAACACAAAGTCTACGCCTGCGAAGTCACCCAT | |
| | CAGGGCCTGAGCTCGCCCGTCACAAAGAGCTTC | |
| | AACAGGGGAGAGTGT | |
| GIPR-0107 | CAGGTTCAGCTGGTGCAGAGCGGTGCAGAAGTG | 159 |
| VH DNA | AAAAAACCTGGTAGCAGCGTGAAAGTGTCATGC | |
| | AAGGCGTCAGGCTATACATTCTCATCATATGCA | |
| | ATCAACTGGGTCAGACAGGCGCCCGGACAGGGA | |
| | CTGGAATGGATGGGGGGCATTGTGCCGATTTTC | |
| | GGAATTGCAAACTATGCGCAGAAATTTCAGGGT | |
| | CGTGTGACCATCACTGCAGATGAATCAACCAGC | |
| | ACCGCCTATATGGAACTGAGCAGCCTGCGTAGC | |
| | GAAGATACCGCAGTATACTATTGCGCCCGTGGA | |
| | AGTGATTATGTGGTCCCCCACTTCGACTATTGG | |
| | GGCCAAGGTACACTGGTCACCGTCAGCAGC | |
| GIPR-0107 | CAGGTTCAGCTGGTGCAGAGCGGTGCAGAAGTG | 160 |
| HC DNA | AAAAAACCTGGTAGCAGCGTGAAAGTGTCATGC | |
| | AAGGCGTCAGGCTATACATTCTCATCATATGCA | |
| | ATCAACTGGGTCAGACAGGCGCCCGGACAGGGA | |
| | CTGGAATGGATGGGGGGCATTGTGCCGATTTTC | |
| | GGAATTGCAAACTATGCGCAGAAATTTCAGGGT | |
| | CGTGTGACCATCACTGCAGATGAATCAACCAGC | |
| | ACCGCCTATATGGAACTGAGCAGCCTGCGTAGC | |
| | GAAGATACCGCAGTATACTATTGCGCCCGTGGA | |
| | AGTGATTATGTGGTCCCCCACTTCGACTATTGG | |
| | GGCCAAGGTACACTGGTCACCGTCAGCAGCGCG | |
| | TCGACCAAGGGCCCATCGGTCTTCCCCCTGGCA | |
| | CCCTCCTCCAAGAGCACCTCTGGGGGCACAGCG | |
| | GCCCTGGGCTGCCTGGTCAAGGACTACTTCCCC | |
| | GAACCGGTGACGGTGTCGTGGAACTCAGGCGCC | |
| | CTGACCAGCGGCGTGCACACCTTCCCGGCTGTC | |
| | CTACAGTCCTCAGGACTCTACTCCCTCAGCAGC | |
| | GTGGTGACCGTGCCCTCCAGCAGCTTGGGCACC | |
| | CAGACCTACATCTGCAACGTGAATCACAAGCCC | |
| | AGCAACACCAAGGTGGACAAGAAAGTTGAGCCC | |
| | AAATCTTGTGACAAAACTCACACATGCCCACCG | |
| | TGCCCAGCACCTGAAGCCGCTGGGGCACCGTCA | |
| | GTCTTCCTCTTCCCCCCAAAACCCAAGGACACC | |
| | CTCATGATCTCCCGGACCCCTGAGGTCACATGC | |
| | GTGGTGGTGGACGTGAGCCACGAAGACCCTGAG | |
| | GTCAAGTTCAACTGGTACGTGGACGGCGTGGAG | |
| | GTGCATAATGCCAAGACAAAGCCGCGGGAGGAG | |
| | CAGTACAACAGCACGTACCGTGTGGTCAGCGTC | |
| | CTCACCGTCCTGCACCAGGACTGGCTGAATGGC | |
| | AAGGAGTACAAGTGCAAGGTCTCCAACAAAGCC | |
| | CTCCCAGCCCCCATCGAGAAAACCATCTCCAAA | |
| | GCCAAAGGGCAGCCCCGAGAACCACAGGTGTAC | |
| | ACCCTGCCCCCATCCCGGGAGGAGATGACCAAG | |
| | AACCAGGTCAGCCTGACCTGCCTGGTCAAAGGC | |
| | TTCTATCCCAGCGACATCGCCGTGGAGTGGGAG | |
| | AGCAATGGGCAGCCGGAGAACAACTACAAGACC | |
| | ACGCCTCCCGTGCTGGACTCCGACGGCTCCTTC | |
| | TTCCTCTATAGCAAGCTCACCGTGGACAAGAGC | |
| | AGGTGGCAGCAGGGGAACGTCTTCTCATGCTCC | |
| | GTGATGCATGAGGCTCTGCACAACCACTACACG | |
| | CAGAAGAGCCTCTCCCTGTCCCCGGGAAAA | |
| GIPR-0107 VL | CAGAGCGTGCTGACACAGCCTCCCAGCGCCTCT | 16 |
| DNA | GGCACCCCTGGCCAGAGAGTGACCATCAGCTGC | |
| | AGCGGATCCAGCAGCAACATCGGCAGCAACTAC | |
| | GTGTACTGGTATCAGCAATTGCCCGGCACCGCC | |
| | CCCAAGCTGCTGATCTACCGGAACAACCAGCGG | |
| | CCCAGCGGCGTGCCCGATCGGTTTAGCGGCAGC | |
| | AAGAGCGGCACAAGCGCCAGCCTGGCCATCAGC | |
| | GGCCTGAGAAGCGAGGACGAGGCGGATTACTAC | |
| | TGCGCCGCCTGGGACGACAGCCTGAGCGGCGTG | |
| | GTGTTTGGAGGCGGCACCAAGCTGACCGTGCTA | |
| GIPR-0107 LC | CAGAGCGTGCTGACACAGCCTCCCAGCGCCTCT | 162 |
| DNA | GGCACCCCTGGCCAGAGAGTGACCATCAGCTGC | |
| | AGCGGATCCAGCAGCAACATCGGCAGCAACTAC | |
| | GTGTACTGGTATCAGCAATTGCCCGGCACCGCC | |
| | CCCAAGCTGCTGATCTACCGGAACAACCAGCGG | |
| | CCCAGCGGCGTGCCCGATCGGTTTAGCGGCAGC | |
| | AAGAGCGGCACAAGCGCCAGCCTGGCCATCAGC | |
| | GGCCTGAGAAGCGAGGACGAGGCGGATTACTAC | |
| | TGCGCCGCCTGGGACGACAGCCTGAGCGGCGTG | |
| | GTGTTTGGAGGCGGCACCAAGCTGACCGTGCTA | |
| | GGTCAGCCCAAGGCTGCCCCCTCGGTCACTCTG | |
| | TTCCCGCCCTCCTCTGAGGAGCTTCAAGCCAAC | |
| | AAGGCCACACTGGTGTGTCTCATAAGTGACTTC | |
| | TACCCGGGAGCCGTGACAGTGGCCTGGAAGGCA | |
| | GATAGCAGCCCCGTCAAGGCGGGAGTGGAGACC | |
| | ACCACACCCTCCAAACAAAGCAACAACAAGTAC | |
| | GCGGCCAGCAGCTATCTGAGCCTGACGCCTGAG | |
| | CAGTGGAAGTCCCACAGAAGCTACAGCTGCCAG | |
| | GTCACGCATGAAGGGAGCACCGTGGAGAAGACA | |
| | GTGGCCCCTACAGAATGTTCA | |

In some embodiments, an anti-GIPR antibody of the disclosure encompasses an antibody that competes for binding to human GIPR with GIP, or binds the same epitope on GIPR, with and as an anti-GIPR antibody comprising (i) a heavy chain variable region having the amino acid sequence set forth in SEQ ID NO: 1, and light chain variable region having the amino acid sequence set forth in SEQ ID NO: 9; (ii) a heavy chain variable region having the amino acid sequence set forth in SEQ ID NO: 14, and light chain variable region having the amino acid sequence set forth in SEQ ID NO: 22; (iii) a heavy chain having the amino acid sequence set forth in SEQ ID NO: 8, and light chain having the amino acid sequence set forth in SEQ ID NO: 13; or (iv) a heavy chain having the amino acid sequence set forth in SEQ ID NO: 21, and light chain having the amino acid sequence set forth in SEQ ID NO: 26.

An anti-GIPR antibody of the present disclosure can encompass monoclonal antibodies, polyclonal antibodies, antibody fragments (e.g., Fab, Fab′, F(ab′)₂, Fv, Fc, etc.), chimeric antibodies, bispecific antibodies, heteroconjugate antibodies, single chain (ScFv), mutants thereof, fusion proteins comprising an antibody fragment (e.g., a domain antibody), humanized antibodies, and any other modified configuration of the immunoglobulin molecule that comprises an antigen binding site of the required specificity, including glycosylation variants of antibodies, amino acid sequence variants of antibodies, and covalently modified antibodies. The antibodies may be murine, rat, human, or any other origin (including chimeric or humanized antibodies). In some embodiments, the anti-GIPR antibody is a monoclonal antibody. In some embodiments, the anti-GIPR antibody is a human or humanized antibody. In some embodiments, anti-GIPR antibody is a chimeric antibody.

The invention also provides CDR portions of anti-GIPR antibody. Determination of CDR regions is well within the skill of the art. It is understood that in some embodiments, CDRs can be a combination of the Kabat and Chothia CDR (also termed “combined CDRs” or “extended CDRs”). In another approach, referred to herein as the “conformational definition” of CDRs, the positions of the CDRs may be identified as the residues that make enthalpic contributions to antigen binding. See, e.g., Makabe et al., 2008, Journal of Biological Chemistry, 283:1156-1166. In general, “conformational CDRs” include the residue positions in the Kabat CDRs and Vernier zones which are constrained in order to maintain proper loop structure for the antibody to bind a specific antigen. Determination of conformational CDRs is well within the skill of the art. In some embodiments, the CDRs are the Kabat CDRs. In other embodiments, the CDRs are the Chothia CDRs. In other embodiments, the CDRs are the extended, AbM, conformational, or contact CDRs. In other words, in embodiments with more than one CDR, the CDRs may be any of Kabat, Chothia, extended, AbM, conformational, contact CDRs or combinations thereof.

In some embodiments, the anti-GIPR antibody described herein comprises an Fc domain. The Fc domain can be derived from IgA (e.g., IgA₁ or IgA₂), IgG, IgE, or IgG (e.g., IgG₁, IgG₂, IgG₃, or IgG₄). In some embodiments, the Fc domain is a human IgG1 Fc domain.

The invention encompasses modifications to the variable regions, the CDRs and the heavy chain and light chain sequences shown in Table 1. For example, the invention includes antibodies comprising functionally equivalent variable regions and CDRs which do not significantly affect their properties as well as variants which have enhanced or decreased activity or affinity. For example, the amino acid sequence may be mutated to obtain an antibody with the desired binding affinity to GIPR. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs.

A modification or mutation may also be made in a framework region or constant region to increase the half-life of an antibody provided herein. See, e.g., PCT Publication No. WO 00/09560. A mutation in a framework region or constant region can also be made to alter the immunogenicity of the antibody, to provide a site for covalent or non-covalent binding to another molecule, or to alter such properties as complement fixation, FcR binding and antibody-dependent cell-mediated cytotoxicity. In some embodiments, no more than one to five conservative amino acid substitutions are made within the framework region or constant region. In other embodiments, no more than one to three conservative amino acid substitutions are made within the framework region or constant region. According to the invention, a single antibody may have mutations in any one or more of the CDRs or framework regions of the variable domain or in the constant region.

In some embodiments, the antibody comprises a modified constant region that has increased or decreased binding affinity to a human Fc gamma receptor, is immunologically inert or partially inert, e.g., does not trigger complement mediated lysis, does not stimulate antibody-dependent cell mediated cytotoxicity (ADCC), or does not activate microglia; or has reduced activities (compared to the unmodified antibody) in any one or more of the following: triggering complement mediated lysis, stimulating ADCC, or activating microglia. Different modifications of the constant region may be used to achieve optimal level or combination of effector functions. See, for example, Morgan et al., Immunology 86:319-324, 1995; Lund et al., J. Immunology 157:4963-9 157:4963-4969, 1996; Idusogie et al., J. Immunology 164:4178-4184, 2000; Tao et al., J. Immunology 143: 2595-2601, 1989; and Jefferis et al., Immunological Reviews 163:59-76, 1998. In some embodiments, the constant region is modified as described in Eur. J. Immunol., 1999, 29:2613-2624; PCT Publication No. WO99/058572.

Modifications also include glycosylated and nonglycosylated polypeptides, as well as polypeptides with other post-translational modifications, such as, for example, glycosylation with different sugars, acetylation, and phosphorylation. Antibodies are glycosylated at conserved positions in their constant regions (Jefferis and Lund, 1997, Chem. Immunol. 65:111-128; Wright and Morrison, 1997, TibTECH 15:26-32). The oligosaccharide side chains of the immunoglobulins affect the protein's function (Boyd et al., 1996, Mol. Immunol. 32:1311-1318; Wittwe and Howard, 1990, Biochem. 29:4175-4180) and the intramolecular interaction between portions of the glycoprotein, which can affect the conformation and presented three-dimensional surface of the glycoprotein (Jefferis and Lund, supra; Wyss and Wagner, 1996, Current Opin. Biotech. 7:409-416). Oligosaccharides may also serve to target a given glycoprotein to certain molecules based upon specific recognition structures. Glycosylation of antibodies has also been reported to affect antibody-dependent cellular cytotoxicity (ADCC). In particular, antibodies produced by CHO cells with tetracycline-regulated expression of β(1,4)-N-acetylglucosaminyltransferase III (GnTIII), a glycosyltransferase catalyzing formation of bisecting GlcNAc, was reported to have improved ADCC activity (Umana et al., 1999, Nature Biotech. 17:176-180).

In some embodiments, the disclosure provides anti-GIPR antibodies containing variations of the variable regions, the CDRs, or the heavy chain and light chain sequences as shown in Table 1, wherein such variant polypeptides share at least about 70%, at least about 75%, at least about 80%, at least about 85%, at least about 87%, at least about 89%, at least about 90%, at least about 91%, at least about 92%, at least about 93%, at least about 94%, at least about 95%, at least about 96%, at least about 97%, at least about 98%, or at least about 99% amino acid sequence identity to any of the amino acid sequences disclosed in Table 1. These amounts are not meant to be limiting and increments between the recited percentages are specifically envisioned as part of the disclosure.

The invention also encompasses fusion proteins comprising one or more components of the anti-GIPR antibodies disclosed herein. In some embodiments, a fusion protein may be made that comprises all or a portion of an anti-GIPR antibody of the invention linked to another polypeptide. In another embodiment, only the variable domains of the anti-GIPR antibody are linked to the polypeptide. In another embodiment, the VH domain of an anti-GIPR antibody linked to a first polypeptide, while the VL domain of an anti-GIPR antibody is linked to a second polypeptide that associates with the first polypeptide in a manner such that the VH and VL domains can interact with one another to form an antigen binding site. In another embodiment, the VH domain is separated from the VL domain by a linker such that the VH and VL domains can interact with one another. The VH-linker-VL antibody is then linked to the polypeptide of interest. In addition, fusion antibodies can be created in which two (or more) single-chain antibodies are linked to one another. This is useful if one wants to create a divalent or polyvalent antibody on a single polypeptide chain, or if one wants to create a bispecific antibody.

### Biological Activity of the Anti-GIPR Antibodies of the Invention

In addition to binding an epitope on GIPR, the anti-GIPR antibodies, of the disclosure can mediate a biological activity. The disclosure includes an isolated antibody that specifically binds GIPR, and mediates at least one detectable activity selected from the following:
(i) binds specifically to human GIPR, and in particularly the N-terminal extra cellular domain of human GIPR;
(ii) binds specifically to cynomolgus monkey GIPR, and in particularly the N-terminal extra cellular domain of monkey GIPR
(iii) reduces, inhibits or neutralizes interaction (e.g., binding) between GIP and soluble GIPR;
(iv) reduces, inhibits or neutralizes interaction (e.g., binding) between GIP and GIPR expressed in various tissues including the pancreas, gut, adipose tissue, vasculature, heart, and brain.

### Polynucleotides Encoding Anti-GIPR Antibodies and Methods of Manufacture

The disclosure also provides polynucleotides encoding any of the antibodies disclosed herein, including antibody portions and modified antibodies described herein. The invention also provides a method of making any of the antibodies and polynucleotides described herein. Polynucleotides can be made and the proteins expressed by procedures known in the art.

If desired, an anti-GIPR antibody of interest may be sequenced and the polynucleotide sequence may then be cloned into a vector for expression or propagation. The sequence encoding the antibody of interest may be maintained in vector in a host cell and the host cell can then be expanded and frozen for future use. Production of recombinant monoclonal antibodies in cell culture can be carried out through cloning of antibody genes from B cells by means known in the art. See, e.g. Tiller et al., 2008, J. Immunol. Methods 329, 112; U.S. Pat. No. 7,314,622.

In some embodiments, provided herein is a polynucleotide comprising a sequence encoding one or both of the heavy chain or the light chain variable regions of an anti-GIPR1 antibody provided herein. The sequence encoding the antibody of interest may be maintained in a vector in a host cell and the host cell can then be expanded and frozen for future use. Vectors (including expression vectors) and host cells are further described herein.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions or substitutions of nucleotides. The resulting mRNA and protein may, but need not, have an altered structure or function. Alleles may be identified using standard techniques (such as hybridization, amplification or database sequence comparison).

In one embodiment, the VH and VL domains or full-length HC or LC, are encoded by separate polynucleotides. Alternatively, both VH and VL, or HC and LC, are encoded by a single polynucleotide.

Polynucleotides complementary to any such sequences are also encompassed by the present disclosure. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present disclosure, and a polynucleotide may, but need not, be linked to other molecules or support materials.

The polynucleotides of this invention can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome.

Suitable cloning vectors may be constructed according to standard techniques, or may be selected from a large number of cloning vectors available in the art. While the cloning vector selected may vary according to the host cell intended to be used, useful cloning vectors will generally have one or more features such as i) the ability to self-replicate, ii) a single target for a particular restriction endonuclease, or iii) may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors are further provided. Expression vectors generally are replicable polynucleotide constructs that contain a polynucleotide according to the invention. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

The invention also provides host cells comprising any of the polynucleotides described herein. Any host cells capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtillis) and yeast (such as S. cerevisae, S. pombe; or K. lactis).

Additionally, any number of commercially and non-commercially available cell lines that express polypeptides or proteins may be utilized in accordance with the present invention. One skilled in the art will appreciate that different cell lines might have different nutrition requirements or might require different culture conditions for optimal growth and polypeptide or protein expression, and will be able to modify conditions as needed.

### Anti-GIPR Antibody Conjugates

GLP-1 is the natural ligand and an agonist of its receptor GLP-1R. Exendin 4 is a naturally occurring peptide that is a GLP-1R agonist. GLP-1R agonists such as GLP-1 analogs and exendin 4 analogs including Liraglutide, Exenatide, Albiglutide, Dulaglutide and Semaglutide, have been known and used to treat metabolic diseases. (Anderson et al., Nature Review Endocrinology, 14, 390-403(2018)). Many GLP1 analogs and Exendin 4 analogs are known in the art. WO2018/136440 and WO 2017112824.

Glucagon (GCG) is a natural polypeptide, a ligand and agonist of GCGR. Glucagon plays an important role in human physiology, such as increasing hepatic glucose production and helping with hepatic lipid metabolism. (Hadersdal et al., Nature Reviews Endocrinology, 19, 321-335 (2023)).

Also provide herein is an anti-GIPR antibody conjugate comprising an anti-GIPR antibody disclosed herein and a polypeptide, wherein the anti-GIPR antibody is conjugated to the polypeptide at a conjugation site through a linker, and the conjugation site is an amino acid residue of the antibody, and the polypeptide is a GLP-1R agonist, a GCGR agonist, or both a GLP1-R agonist and a GCGR agonist, that is a dual agonist of GLP-1R and GCGR.

In some embodiments of the anti-GIPR antibody conjugate, the polypeptide is a GLP-1R agonist. In some embodiments, the GLP-1R agonist is a GLP-1 analog. An GLP-1 analog as used herein refers to a polypeptide that is an agonist of GLP-1R and comprises an amino acid sequence that is at least about 50%, at least about 60%, at least about 70%, at least about 75%, at least about 80%, at least about 85%, at least about 90%, or at least about 95% identical to the wild type GLP-1 (7-37) or GLP-1 (7-36). In some embodiments, the GLP-1R agonist is an exendin 4 analog. An exendin 4 analog, as used herein, refers to a polypeptide that is an agonist of GLP-1R and comprises an amino acid sequence that is at least about 50%, at least about 60%, at least about 70%, at least about 75%, at least about 80%, at least about 85%, at least about 90%, or at least about 95% identical to the wild type exendin 4.

In some embodiments of the anti-GIPR antibody conjugate, the polypeptide is a GLP-1R agonist comprising the amino acid sequences selected from the amino acid sequences in Table 2. Table 2 provides the amino acid sequences of wild type GLP-1, exemplified GLP-1 analogs, and exendin 4.

TABLE 2
GLP-1, Selected GLP-1 Analogs and Exendin 4 Sequences

| Peptide | Sequence | SEQ ID NO |
|---|---|---|
| GLP-1 | HAEGTFTSDVSSYLEGQAAKEFIAWLVKGRG | 40 |
| GLP-1 (7-37) | HAEGTFTSDVSSYLEGQAAKEFIAWLVKGRG | 41 |
| GLP-1 (7-36) | HAEGTFTSDVSSYLEGQAAKEFIAWLVKGRG | 42 |
| GLP1(7-37) | H-Aib-EGTFTSDYSSYLEEQAAKEFIAWLVKGRG | 43 |
| [Aib8; Tyr16; Glu22] | | |
| GLP1(7-37) | H-Aib-EGTFTSDYSSYLE-Aib-QAAKEFIAWLVKGRG | 44 |
| [Aib8; Tyr16; Aib22] | | |
| GLP1(7-37) | H-Aib-EGTFTSDVSSYLE-Aib-QAAKEFIAWLVKGRG | 45 |
| [Aib8; AiB22] | | |
| GLP1(7-37) | H-Aib-EGTFTSDVSSYLEEQAAKEFIAWLVKGRG | 46 |
| [Aib8;Glu22] | | |
| GLP1(7-35) | H-Aib-EGTFTSDYSSYLEEQAAKEFIAWLVKG | 47 |
| [Aib8;Tyr16;Glu22] | | |
| GLP1(7-35) | H-Aib-EGTFTSDYSSYLE-AIB-QAAKEFIAWLVKG | 48 |
| [Aib8; Tyr16; Aib22] | | |
| GLP1(7-36) [Aib8; | H-Aib-EGTFTSDYSSYLEEQAAKEFIAWLVKGG | 49 |
| Tyr16; Glu22; | | |
| Gly36] | | |
| GLP1(7-36) [Aib8; | H-Aib-EGTFTSDYSSYLE-AIB-QAAKEFIAWLVKGG | 50 |
| Tyr16; | | |
| Aib22; Gly36] | | |
| GLP1(7-37) [Aib8; | H-Aib-EGTFTSDYSSYLEEQAAKEFIAWLVRGGG | 51 |
| Tyr16; Glu22; | | |
| Arg34; Gly36] | | |
| GLP1(7-37) [Aib8; | H-Aib-EGTFTSDYSSYLE-Aib-QAAKEFIAWLVRGGG | 52 |
| Tyr16; AiB22; | | |
| Arg34; Gly36] | | |
| GLP1(7-37) [Aib8; | H-Aib-EGTFTSDYSSYLEEQAAKEFIAWLVRGRG | 53 |
| Tyr16; Glu22; | | |
| Arg34] | | |
| GLP1(7-37) [Aib8; | H-Aib-EGTFTSDYSSYLE-Aib-QAAKEFIAWLVRGRG | 54 |
| Tyr16; AiB22; | | |
| Arg34] | | |
| GLP1(7-37) | HGEGTFTSDYSSYLEEQAAKEFIAWLVKGGG | 55 |
| [Gly8; Tyr16; | | |
| Glu22; Gly36] | | |
| GLP1(7-37) [Gly8; | HGEGTFTSDYSSYLE-Aib-QAAKEFIAWLVKGGG | 56 |
| Tyr16; | | |
| Aib22; Gly36] | | |
| GLP1(7-37) [Aib8; | H-Aib-EGTFTSDYSSYLEAQAAKEFIAWLVKGGG | 57 |
| Tyr16; Ala22; | | |
| Gly36] | | |
| GLP1(7-37) [Aib8; | H-Aib-EGTFTSDYSSYLEEQAAKEFIAWLVKGGG | 58 |
| Tyr16; Glu22; | | |
| Gly36] | | |
| GLP1(7-37) [Alb8; | H-Aib-EGTFTSDVSSYLE-Aib-QAAKEFIAWLVKGGG | 59 |
| Aib22; Gly36] | | |
| GLP1(7-35) [Aib8; | H-Aib-EGTFTSDVSSYLE-Aib-QAAKEFIAWLVKG | 60 |
| AiB22] | | |
| Exendin 4 | HGEGTFTSDL SKQMEEEAVR LFIEWLKNGG | 61 |
| | PSSGAPPPS | |

In some embodiments of the anti-GIPR antibody conjugate, the polypeptide is a GLP-1R agonist comprising an amino acid sequence selected from the amino acid sequences in Table 2. In some embodiments, the polypeptide is a GLP-1R agonist having an acid sequence selected from the amino acid sequences in Table 2.

In some embodiments of the anti-GIPR antibody conjugate, the polypeptide is a GCGR agonist. In some embodiments, the GCGR agonist is a GCG analog. An GCG analog, used herein, refers to a polypeptide that is an agonist of GCGR and comprises an amino acid sequence that is at least about 50%, at least about 60%, at least about 70%, at least about 75%, at least about 80%, at least about 85%, at least about 90%, or at least about 95% identical to the wild type GCG.

In some embodiments of the anti-GIPR antibody conjugate, the polypeptide is a dual agonist that is both a GLP-1R agonist and a GCGR agonist. In some embodiments, the dual agonist is both (i) a GLP-1 (7-37) analog or an exendin 4 analog and (ii) a GCG analog.

Table 3 provides exemplified dual agonist of GLP-1R and GCGR that is both a GLP-1 analog and a GCG analog.

TABLE 3
Sequences of GCG, GCGR/GLP-1R Dual Agonist, GLP-1 Analogs and
GCG Analogs

| Peptide | Sequence | SEQ ID NO |
|---|---|---|
| Glucagon | HSQGTGTSDYSKYLDSRRAQDFVQWLMNT | 62 |
| mazdutide | H[Aib]QGTFTSDYSKYLDEKKAKEFVEWLLEGGPSSG | 63 |
| Survodutide | H-X1-QGTFTSDYSKYLDERAAKDFIKWLESA | 64 |
| Pemvidutide | H-Aib-QGTFTSDYSKYLDEKAAKEFIQWLLQT | 65 |
| NN1177 | H-Aib-QGTFTSDLSKYLESKRAREFVQWLLKT | 66 |
| NN1151 | H-X1-QGTFTSDLSKYLDLRRARDFVQWLLKT | 67 |
| NN1359 | X2-Aib-HGTFTSDVSKYLEEQAAREFIAWLLKT | 68 |
| OPK88003 | H-Aib-QGTFTSDYSKYLDSKKAQEFVEWLLN-Aib- | 69 |
| | GRNRNNIA-NH-X3 | |
| Peptide 15 | HSQGTFTSDYSKYLDEQAAKEFIAWLMNT | 70 |
| Retatrutide | Y-Aib-QGTFTSDYSI-(a-Me-Leu)-LDKKAQ- | 71 |
| | Aib-AFIEYLLEGGPSSGAPPPS-NH2 | |
| MK-1462 | H[dSer]QGTFTSDKSKYLDARAAQDFVQWLLDT | 72 |

In some embodiments of the anti-GIPR antibody conjugate, the polypeptide is a dual agonist of GCGR/GLP-1R, comprising an amino acid sequence selected from the amino acid sequences in Table 3. In some embodiments, the polypeptide is a dual agonist of GCGR/GLP-1R, having the amino acid sequence selected from the amino acid sequences in Table 3.

Table 4 provides the amino acid sequences of some additional GCGR/GLP-1R dual agonist.

TABLE 4
Sequences of Additional GCGR/GLP-1R Dual Agonists.

| Name | Sequence | SEQ ID NO |
|---|---|---|
| GGM1_1 | [X2][Aib]QGTFTSDYSKYLDEKKAKEFVEWLLEGGPSSG | 73 |
| GGM1_2 | [X4][Aib]QGTFTSDYSKYLDEKKAKEFVEWLLEGGPSSG | 74 |
| GGM1_3 | F[Aib]QGTFTSDYSKYLDEKKAKEFVEWLLEGGPSSG | 75 |
| GGM1_4 | Y[Aib]QGTFTSDYSKYLDEKKAKEFVEWLLEGGPSSG | 76 |
| GGM1_5 | [X5][Aib]QGTFTSDYSKYLDEKKAKEFVEWLLEGGPSSG | 77 |
| GGM1_6 | [X6][Aib]QGTFTSDYSKYLDEKKAKEFVEWLLEGGPSSG | 78 |
| GGM1_7 | [X7][Aib]QGTFTSDYSKYLDEKKAKEFVEWLLEGGPSSG | 79 |
| GGM1_8 | HAQGTFTSDYSKYLDEKKAKEFVEWLLEGGPSSG | 80 |
| GGM1_9 | HGQGTFTSDYSKYLDEKKAKEFVEWLLEGGPSSG | 81 |
| GGM1_10 | HSQGTFTSDYSKYLDEKKAKEFVEWLLEGGPSSG | 82 |
| GGM1_11 | H[X1]QGTFTSDYSKYLDEKKAKEFVEWLLEGGPSSG | 83 |
| GGM1_12 | H[X8]QGTFTSDYSKYLDEKKAKEFVEWLLEGGPSSG | 84 |
| GGM1_13 | H[dSer]QGTFTSDYSKYLDEKKAKEFVEWLLEGGPSSG | 85 |
| GGM1_14 | H[dAla]QGTFTSDYSKYLDEKKAKEFVEWLLEGGPSSG | 86 |
| GGM1_15 | H[Aib]EGTFTSDYSKYLDEKKAKEFVEWLLEGGPSSG | 87 |
| GGM1_16 | H[Aib]HGTFTSDYSKYLDEKKAKEFVEWLLEGGPSSG | 88 |
| GGM1_17 | H[Aib]Q[dAla]TFTSDYSKYLDEKKAKEFVEWLLEGGPSSG | 89 |
| GGM1_18 | H[Aib]QGT[X9]TSDYSKYLDEKKAKEFVEWLLEGGPSSG | 90 |
| GGM1_20 | H[Aib]QGTFTSDVSKYLDEKKAKEFVEWLLEGGPSSG | 91 |
| GGM1_21 | H[Aib]QGTFTSDKSKYLDEKKAKEFVEWLLEGGPSSG | 92 |
| GGM1_22 | H[Aib]QGTFTSDLSKYLDEKKAKEFVEWLLEGGPSSG | 93 |
| GGM1_23 | H[Aib]QGTFTSDASKYLDEKKAKEFVEWLLEGGPSSG | 94 |
| GGM1_24 | H[Aib]QGTFTSDY[X10]KYLDEKKAKEFVEWLLEGGPSSG | 95 |
| GGM1_25 | H[Aib]QGTFTSDYAKYLDEKKAKEFVEWLLEGGPSSG | 96 |
| GGM1_26 | H[Aib]QGTFTSDYSSYLDEKKAKEFVEWLLEGGPSSG | 97 |
| GGM1_27 | H[Aib]QGTFTSDYSEYLDEKKAKEFVEWLLEGGPSSG | 98 |
| GGM1_28 | H[Aib]QGTFTSDYSIYLDEKKAKEFVEWLLEGGPSSG | 99 |
| GGM1_29 | H[Aib]QGTFTSDYSAYLDEKKAKEFVEWLLEGGPSSG | 100 |
| GGM1_30 | H[Aib]QGTFTSDYSKQLDEKKAKEFVEWLLEGGPSSG | 101 |
| GGM1_31 | H[Aib]QGTFTSDYSK[X11]LDEKKAKEFVEWLLEGGPSSG | 102 |
| GGM1_32 | H[Aib]QGTFTSDYSK[X12]LDEKKAKEFVEWLLEGGPSSG | 103 |
| GGM1_33 | H[Aib]QGTFTSDYSKYMDEKKAKEFVEWLLEGGPSSG | 104 |
| GGM1_34 | H[Aib]QGTFTSDYSKYLEEKKAKEFVEWLLEGGPSSG | 105 |
| GGM1_35 | H[Aib]QGTFTSDYSKYLDGKKAKEFVEWLLEGGPSSG | 106 |
| GGM1_36 | H[Aib]QGTFTSDYSKYLDSKKAKEFVEWLLEGGPSSG | 107 |
| GGM1_37 | H[Aib]QGTFTSDYSKYLD[Aib]KKAKEFVEWLLEGGPSSG | 108 |
| GGM1_38 | H[Aib]QGTFTSDYSKYLDAKKAKEFVEWLLEGGPSSG | 109 |
| GGM1_39 | H[Aib]QGTFTSDYSKYLD[X13]KKAKEFVEWLLEGGPSSG | 110 |
| GGM1_40 | H[Aib]QGTFTSDYSKYLDEQKAKEFVEWLLEGGPSSG | 111 |
| GGM1_41 | H[Aib]QGTFTSDYSKYLDERKAKEFVEWLLEGGPSSG | 112 |
| GGM1_42 | H[Aib]QGTFTSDYSKYLDEEKAKEFVEWLLEGGPSSG | 113 |
| GGM1_43 | H[Aib]QGTFTSDYSKYLDEAKAKEFVEWLLEGGPSSG | 114 |
| GGM1_44 | H[Aib]QGTFTSDYSKYLDEKAAKEFVEWLLEGGPSSG | 115 |
| GGM1_45 | H[Aib]QGTFTSDYSKYLDEKRAKEFVEWLLEGGPSSG | 116 |
| GGM1_46 | H[Aib]QGTFTSDYSKYLDEKSAKEFVEWLLEGGPSSG | 117 |
| GGM1_47 | H[Aib]QGTFTSDYSKYLDEKKVKEFVEWLLEGGPSSG | 118 |
| GGM1_48 | H[Aib]QGTFTSDYSKYLDEKKLKEFVEWLLEGGPSSG | 119 |
| GGM1_49 | H[Aib]QGTFTSDYSKYLDEKK[Aib]KEFVEWLLEGGPSSG | 120 |
| GGM1_50 | H[Aib]QGTFTSDYSKYLDEKKAREFVEWLLEGGPSSG | 12″ |
| GGM1_51 | H[Aib]QGTFTSDYSKYLDEKKAQEFVEWLLEGGPSSG | 122 |
| GGM1_52 | H[Aib]QGTFTSDYSKYLDEKKANEFVEWLLEGGPSSG | 123 |
| GGM1_53 | H[Aib]QGTFTSDYSKYLDEKKA[Aib]EFVEWLLEGGPSSG | 124 |
| GGM1_54 | H[Aib]QGTFTSDYSKYLDEKKAKDFVEWLLEGGPSSG | 125 |
| GGM1_55 | H[Aib]QGTFTSDYSKYLDEKKAKLFVEWLLEGGPSSG | 126 |
| GGM1_56 | H[Aib]QGTFTSDYSKYLDEKKAKQFVEWLLEGGPSSG | 127 |
| GGM1_57 | H[Aib]QGTFTSDYSKYLDEKKAKEFIEWLLEGGPSSG | 128 |
| GGM1_58 | H[Aib]QGTFTSDYSKYLDEKKAKEFAEWLLEGGPSSG | 129 |
| GGM1_59 | H[Aib]QGTFTSDYSKYLDEKKAKEFVAWLLEGGPSSG | 130 |
| GGM1_60 | H[Aib]QGTFTSDYSKYLDEKKAKEFVKWLLEGGPSSG | 131 |
| GGM1_61 | H[Aib]QGTFTSDYSKYLDEKKAKEFVQWLLEGGPSSG | 132 |
| GGM1_62 | H[Aib]QGTFTSDYSKYLDEKKAKEFVEWLMNT | 133 |
| GGM1_63 | H[Aib]QGTFTSDYSKYLDEKKAKEFVEWLESA | 134 |
| GGM1_64 | H[Aib]QGTFTSDYSKYLDEKKAKEFVEWLEAGG | 135 |
| GGM1_65 | H[Aib]QGTFTSDYSKYLDEKKAKEFVEWLLQT | 136 |
| GGM1_66 | H[Aib]QGTFTSDYSKYLDEKKAKEFVEWLLKT | 137 |
| GGM1_67 | H[Aib]QGTFTSDYSKYLDEKKAKEFVEWLLNTGRNRNIA | 138 |
| GGM1_68 | H[Aib]QGTFTSDYSKYLDEKKAKEFVEWLVKGRG | 139 |
| GGM1_69 | H[Aib]QGTFTSDYSKYLDEKKAKEFVEWLLNT | 140 |
| GGM1_70 | H[Aib]QGTFTSDYSKYLDEKKAKEFVEWLMNTKRNRNIA | 141 |
| GGM1_71 | H[Aib]QGTFTSDYSKYLDEKKAKEFVEWLLDT | 142 |

In some embodiments of the anti-GIPR antibody conjugate, the polypeptide is a dual agonist of GCGR/GLP-1R, comprising an amino acid sequence selected from the amino acid sequences in Table 4. In some embodiments, the polypeptide is a dual agonist of GCGR/GLP-1R, having the amino acid sequence selected from any one of the amino acid sequences set forth in Table 4.

Also provided in the disclosure is a polypeptide that is a dual agonist of GLP-1R and GCGR. In some embodiments, the polypeptide comprises any one of the amino acid sequences set forth in Table 4. In some embodiments, the polypeptide has the amino acid sequence as any one of the amino acid sequences set forth in Table 4.

Some unnatural amino acids are indicated in Table 3 and Table 4 as the following:
Aib: 2-Aminoisobutyric Acid
dAla: D-Alanine
dSer: D-Serine
X1: 1-amino-1-cyclobutanecarboxylic acid
X2: Deamino-histidine
X3: 4-(((1S)-2-amino-1-((2,5-dioxo-1-(13-oxo-2,5,8-trioxa-12-azatetradecan-14-yl)pyrrolidin-3-yl)thio)-2-oxoethyl)amino)-1-(2,5-dioxo-1-(13-oxo-2,5,8-trioxa-12-azapentadecan-15-yl)pyrrolidin-3-yl)-4-oxobutan-2-ide
X4: 3-(1H-Benzimidazol-5-yl)propanoic acid
X5: 3-(1H-Pyrazol-4-yl)propanoic acid
X6: 3-(1H-Pyrazol-4-yl)-L-alanine
X7: 3-(1H-Benzimidazole-5-yl)-L-alanine
X8: 2-Methyl-L-proline
X9: alpha methyl L-phenylalanine
X10: 5-Phenyl-L-norvaline
X11: alpha methyl L-2-flurophenylalanine
X12: alpha methyl L-Leucine
X13: 2-Methyl-L-glutamic acid

In some embodiments of the anti-GIPR antibody conjugate, an amino acid residue of an anti-GIPR antibody is a cysteine residue or mutated to a cysteine residue to form the conjugation site. In some embodiments, the conjugation site is 118C, 272C, 290C, 380C or 443C of the antibody heavy chain. In some embodiments, the conjugation site is 183C of the antibody light chain, if the light chain is a Kappa light chain. In some embodiments, the conjugation site is 205C of the antibody light chain, if the light chain is a Lambda light chain. All positions are based on EU numbering. See Tumey et al. AAPS J 2017 July; 19(4):1123-1135, WO 2012/125973 A2, and Junutula, J., Raab, H., Clark, S. et al.. Nat Biotechnol 26, 925-932 (2008).

In some embodiments of the anti-GIPR antibody conjugate, the polypeptide is connected to the linker from the N terminus of the polypeptide. In some embodiments, the polypeptide is connected to the linker from the C terminus of the polypeptide.

In some embodiments of the anti-GIPR antibody conjugate, the linker is a 1-30 amino acid peptidyl linker, optionally pegylated, or a PEG₃₋₂₀-K(AcBr)—NH₂ linker. In some embodiments, the linker is selected from the group consisting of (GGGGS)₃, PEG12-K(AcBr)—NH₂ and PEG24-K(AcBr)—NH₂.

### Biological Activity of GIPR Antibody Conjugates of the Invention

In vitro activities of the anti-GIPR antibody -GLP-1R agonist conjugates are primarily being assessed using (1) functional antagonist mode assays measuring the ability of the conjugate to block GIP-induced cAMP production in cells expressing human GIPR, and (2) functional agonist mode assays measuring the ability of conjugates to stimulate cAMP production, beta-arrestin recruitment or insulin secretion in cells expressing human GLP1R and/or human GIPR.

In vitro activities the anti-GIPR antibody—GLP1R/GCGR dual agonist conjugate will primarily be assessed using: (1) functional antagonist mode assays measuring the ability of conjugates to block GIP-induced cAMP production in CHO cells expressing human GIPR; and (2) functional agonist mode assay measuring the ability of conjugates to stimulate cAMP production, beta-arrestin recruitment or insulin secretion in cells expressing human GLP1R or GCGR in the presence or absence of human GIPR.

### Pharmaceutical Compositions

In another embodiment, the invention comprises pharmaceutical compositions. A “pharmaceutical composition” refers to a mixture of an antibody the invention and one or excipient. Pharmaceutical compositions of this invention may be in a variety of forms. These include, for example, liquid, semi-solid and solid dosage forms, such as liquid solutions (e.g., injectable and infusible solutions), dispersions or suspensions, and lyophilized powders. The form depends on the intended mode of administration and therapeutic application.

Other excipients and modes of administration known in the pharmaceutical art may also be used. Pharmaceutical compositions of the invention may be prepared by any of the well-known techniques of pharmacy, such as effective formulation and administration procedures. The above considerations in regard to effective formulations and administration procedures are well known in the art and are described in standard textbooks. Formulation of drugs is discussed in, for example, Hoover, John E., Remington's Pharmaceutical Sciences, Mack Publishing Co., Easton, Pennsylvania, 1975; Liberman et al., Eds., Pharmaceutical Dosage Forms, Marcel Decker, New York, N.Y., 1980; and Kibbe et al., Eds., Handbook of Pharmaceutical Excipients (3^{rd} Ed.), American Pharmaceutical Association, Washington, 1999.

Acceptable excipients are nontoxic to recipients at the dosages and concentrations employed, and may comprise buffers such as phosphate, citrate, and other organic acids; salts such as sodium chloride; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens, such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); or non-ionic surfactants such as TWEEN™, PLURONICS™ or polyethylene glycol (PEG).

### Therapeutic, Diagnostic, and Other Methods

The antibodies and the antibody conjugates of the present invention are useful in various applications including, but are not limited to, therapeutic treatment methods and diagnostic treatment methods. In one embodiment, the antibodies and antibody conjugates disclosed herein are selective toward GIPR. In some embodiments, the antibodies and antibody conjugates disclosed herein do not have functional activity at human GLP-1R. In some embodiments, the antibodies and antibody conjugates disclosed herein do not have functional activity at human GCGR.

In one aspect, the invention provides a method for treating a GIPR-related condition, disease, or disorder, such as a metabolic disease, especially obesity and diabetes. In some embodiments, the method comprises administering to the subject in need thereof an effective amount of a pharmaceutical composition comprising an anti-GIPR antibody or an anti-GIPR antibody conjugate as described herein.

The GIPR-related condition, disease, or disorder includes one selected from diabetes [e.g. Type 1 diabetes mellitus (T1D), Type 2 diabetes mellitus (T2DM), including pre-diabetes], idiopathic T1D (Type 1b), latent autoimmune diabetes in adults (LADA), early-onset T2DM (EOD), youth-onset atypical diabetes (YOAD), maturity onset diabetes of the young (MODY), malnutrition-related diabetes, gestational diabetes, hyperglycemia, insulin resistance, hepatic insulin resistance, impaired glucose tolerance, diabetic neuropathy, diabetic nephropathy, kidney disease [e.g., acute kidney disorder, tubular dysfunction, proinflammatory changes to the proximal tubules, or chronic kidney disease (CKD)], diabetic retinopathy, adipocyte dysfunction, visceral adipose deposition, sleep apnea [e.g. obstructive sleep apnea (OSA)], obesity (including hypothalamic obesity and monogenic obesity) and related comorbidities (e.g., osteoarthritis and urine incontinence), eating disorders (including binge eating syndrome, bulimia nervosa, and syndromic obesity such as Prader-Willi and Bardet-Biedl syndromes), weight gain such as weight gain caused by use of other agents (e.g., caused by use of steroids and/or antipsychotics, or caused by treatment of depression, or caused by use of agents on cognitive function), excessive sugar craving, dyslipidemia [including hyperlipidemia, hypertriglyceridemia, increased total cholesterol, high LDL (low-density lipoprotein) cholesterol, and low HDL (high-density lipoprotein) cholesterol], hyperinsulinemia, nonalcoholic fatty liver disease [NAFLD, including related diseases such as steatosis, nonalcoholic steatohepatitis (NASH), fibrosis, cirrhosis, and hepatocellular carcinoma], cardiovascular disease, atherosclerosis (including coronary artery disease), peripheral vascular disease, hypertension, endothelial dysfunction, impaired vascular compliance, heart failure [e.g. congestive heart failure, heart failure with preserved ejection fraction (HFpEF), heart failure with reduced ejection fraction (HFrEF)], myocardial infarction (e.g. necrosis and apoptosis), stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease, macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, polycystic ovary syndrome (PCOS), and addiction (e.g., addition to alcohol, nicotine, and/or drug).

In another aspect, the invention further provides anti-GIPR antibody, an anti-GIPR antibody conjugate, or pharmaceutical composition thereof as described herein for use in the described method of treating a GIPR-related condition, disease, or disorder, such as a metabolic disease, especially obesity and diabetes. The invention also provides the use of the anti-GIPR antibody or an anti-GIPR antibody conjugate as described herein in the manufacture of a medicament for treating such condition, disease or disorder.

In another aspect, provided is a method of one or more of detecting, diagnosing, or monitoring a GIPR-related condition, disease, or disorder, such as a metabolic disease, especially obesity and diabetes. For example, the anti-GIPR antibody or the anti-GIPR antibody conjugate as described herein can be labeled with a detectable moiety such as an imaging agent and an enzyme-substrate label. The antibodies as described herein can also be used for in vivo in vitro and ex vivo diagnostic assays, prognosis assays such as in vivo imaging (e.g., PET or SPECT), or a staining reagent.

In some embodiments, an antibody or antibody conjugate disclosed herein can be used to treat diabetes. In one embodiment, the diabetes is selected from the group consisting of: type 1 diabetes, type 2 diabetes, pre-diabetes, idiopathic type 1b diabetes, latent autoimmune diabetes in adults (LADA), early-onset type 2 diabetes (EOD), youth-onset atypical diabetes (YOAD), maturity onset diabetes of the young (MODY), malnutrition-related diabetes, and gestational diabetes.

In some embodiments, an antibody or antibody conjugate disclosed herein can be used to treat obesity. In some embodiments, an antibody or antibody conjugate disclosed herein can be used to treat a condition associated with obesity, for example, type 2 diabetes, heart disease, high blood pressure, osteoarthritis, sleep apnea, stroke, fatty liver disease, metabolic syndrome, high cholesterol, kidney disease, liver disease, depression, acute pancreatitis, and cardiovascular disease. In some embodiments, a pharmaceutical composition disclosed herein can be used to treat hyperlipidemia, hypertriglyceridemia, increased total cholesterol, high LDL cholesterol, low HDL cholesterol, and hyperinsulinemia. In some embodiments, an antibody or antibody conjugate disclosed herein can be used to treat a weight-related comorbidity. In one embodiment, the weight-related comorbidity is hypertension, type 2 diabetes, or dyslipidemia.

In one embodiment, an antibody or antibody conjugate disclosed herein can be administered to a subject to reduce the body weight of the subject by at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, or at least about 20%. In one embodiment, an antibody or antibody conjugate disclosed herein can be administered to a subject to reduce the body weight of the subject by at least about 5%. In one embodiment, a pharmaceutical composition disclosed herein can be administered to a subject to reduce the body weight of the subject by at least about 10%. In one embodiment, an antibody or antibody conjugate disclosed herein can be administered to a subject to reduce the body weight of the subject by at least about 15%.

In one embodiment, an antibody or antibody conjugate disclosed herein can be administered to a subject to reduce the BMI of the subject by at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 16%, at least about 17%, at least about 18%, at least about 19%, or at least about 20%. In one embodiment, an antibody or antibody conjugate disclosed herein can be administered to a subject to reduce the BMI of the subject by at least about 5%. In one embodiment, an antibody or antibody conjugate disclosed herein can be administered to a subject to reduce the BMI of the subject by at least about 10%. In one embodiment, an antibody or antibody conjugate disclosed herein can be administered to a subject to reduce the BMI of the subject by at least about 15%.

In some embodiments, an antibody or antibody conjugate disclosed herein can be used for weight management of a subject. In some embodiments, the weight management is chronic weight management. In some embodiments, the subject to be treated is overweight [i.e. having a body mass index (BMI) of 27 kg/m² or greater] when the weight management treatment is initiated. In some further embodiments, the subject to be treated is adult. In some yet further embodiments, the adult subject to be treated has an initial BMI of 27 kg/m² or greater in the presence of at least one weight-related comorbid condition (e.g., hypertension, T2DM, or dyslipidemia).

In some embodiments, the subject to be treated is obese (i.e. having an BMI of 30 kg/m² or greater) when the weight management treatment is initiated. In some further embodiments, the subject to be treated is adult. In some embodiments, the subject to be treated in a pediatric patient. In some further embodiments, the pediatric patient to be treated is aged 12 years or older with an initial BMI at the 95^{th} percentile or greater for age and sex (obese pediatric patient).

With respect to all methods described herein, reference to anti-GIPR antibody also includes pharmaceutical compositions comprising the anti-GIPR antibody and one or more additional agents.

### Administration and Dosing

Typically, an antibody or antibody conjugate of the invention is administered in an amount effective to treat a condition as described herein. In some embodiments, the antibodies of the invention can be administered as an antibody per se, or alternatively, as a pharmaceutical composition containing the antibody. In some embodiments, the antibody conjugates of the invention can be administered as an antibody conjugate per se, or alternatively, as a pharmaceutical composition containing the antibody conjugate.

The antibodies or antibody conjugates of the invention are administered by any suitable route in the form of a pharmaceutical composition adapted to such a route, and in a dose effective for the treatment intended.

In some embodiments, the antibodies or antibody conjugates of the disclosure may be administered parenterally, for example directly into the bloodstream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors, and infusion techniques.

In another embodiment, the antibodies or antibody conjugates of the invention may also be administered topically to the skin or mucosa, that is, dermally or transdermally. In another embodiment, the antibodies or antibody conjugates of the invention can also be administered intranasally or by inhalation. In another embodiment, the antibodies or antibody conjugates of the invention may be administered rectally or vaginally. In another embodiment, the antibodies or antibody conjugates of the invention may also be administered directly to the eye or ear.

The dosage regimen for the antibodies or antibody conjugates of the invention or compositions containing said antibodies or antibody conjugates is based on a variety of factors, including the type, age, weight, sex and medical condition of the subject; the severity of the condition; the route of administration; and the activity of the particular antibody employed. Thus, the dosage regimen may vary widely.

In some embodiments, the initial body mass index (BMI) of a subject administered with a pharmaceutical composition comprising an antibody or antibody conjugate disclosed herein is at least about 24 kg/m². In some embodiments, the initial BMI of a subject administered with a pharmaceutical composition comprising an antibody or antibody conjugate disclosed herein is from about 24 kg/m² to about 30 kg/m². In some embodiments, the initial BMI of a subject administered with a pharmaceutical composition comprising an antibody or antibody conjugate disclosed herein is at least about 27 kg/m². In some embodiments, the initial BMI of a subject administered with a pharmaceutical composition comprising an antibody or antibody conjugate disclosed herein is at least about 30 kg/m². In some embodiments, the initial BMI of a subject administered with a pharmaceutical composition comprising an antibody or antibody conjugate disclosed herein is from about 30.0 kg/m² to about 45 kg/m². As used herein, a human with a BMI of 30 kg/m² or greater is considered obese, and a human with a BMI of from about 25.0 to about 29.9 kg/m² is considered overweight.

In one embodiment, a dose of an antibody or antibody conjugate of the invention is typically from about 0.01 to about 100 mg/kg (i.e., mg antibody of the invention per kg body weight) for the treatment of the indicated conditions discussed herein. In another embodiment, a dose of the antibody or antibody conjugate of the invention is from about 0.1 to about 50 mg/kg, and in another embodiment, from about 0.5 to about 30 mg/kg.

In some embodiments, a pharmaceutical composition comprising an antibody or antibody conjugate disclosed herein can be administered in an amount of from about 1 mg to about 500 mg, from about 1 mg to about 50 mg, from about 50 mg to about 100 mg, from about 100 mg to about 150 mg, from about 150 mg to about 200 mg, from about 200 mg to about 250 mg, or from about 250 mg to about 500 mg. In some embodiments, a pharmaceutical composition comprising an antibody or antibody conjugate disclosed herein can be administered in an amount of from about 1 mg to about 50 mg. In some embodiments, a pharmaceutical composition comprising an antibody or antibody conjugate disclosed herein can be administered in an amount of from about 50 mg to about 100 mg. In some embodiments, a pharmaceutical composition comprising an antibody or antibody conjugate disclosed herein can be administered in an amount of from about 100 mg to about 150 mg. In some embodiments, a pharmaceutical composition comprising an antibody or antibody conjugate disclosed herein can be administered in an amount of from about 150 mg to about 200 mg.

In some embodiments, a pharmaceutical composition comprising an antibody or antibody conjugate disclosed herein can be administered in an amount of about 1 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, or about 500 mg. In some embodiments, a pharmaceutical composition comprising an antibody or antibody conjugate disclosed herein can be administered in an amount of about 20 mg. In some embodiments, a pharmaceutical composition comprising an antibody or antibody conjugate disclosed herein can be administered in an amount of about 50 mg. In some embodiments, a pharmaceutical composition comprising an antibody or antibody conjugate disclosed herein can be administered in an amount of about 80 mg. In some embodiments, a pharmaceutical composition comprising an antibody or antibody conjugate disclosed herein can be administered in an amount of about 100 mg. In some embodiments, a pharmaceutical composition comprising an antibody or antibody conjugate disclosed herein can be administered in an amount of about 120 mg. In some embodiments, a pharmaceutical composition comprising an antibody or antibody conjugate disclosed herein can be administered in an amount of about 150 mg.

In some embodiments, a pharmaceutical composition can comprise an antibody or antibody conjugate disclosed herein in a concentration of from about 0.1 mg/mL to about 100 mg/mL, from about 0.1 mg/mL to about 25 mg/mL, from about 25 mg/mL to about 50 mg/mL, from about 50 mg/mL to about 75 mg/mL, or from about 75 mg/mL to about 100 mg/mL. In some embodiments, a pharmaceutical composition can comprise an antibody or antibody conjugate disclosed herein in a concentration of from about 0.1 mg/mL to about 25 mg/mL. In some embodiments, a pharmaceutical composition can comprise an antibody or antibody conjugate disclosed herein in a concentration of from about 25 mg/mL to about 50 mg/mL. In some embodiments, a pharmaceutical composition can comprise an antibody or antibody conjugate disclosed herein in a concentration of from about 50 mg/mL to about 75 mg/mL. In some embodiments, a pharmaceutical composition can comprise an antibody or antibody conjugate disclosed herein in a concentration of from about 75 mg/mL to about 100 mg/mL.

In some embodiments, a pharmaceutical composition can comprise an antibody or antibody conjugate disclosed herein in a concentration of about 0.5 mg/mL, about 1 mg/mL, about 5 mg/mL, about 10 mg/mL, about 15 mg/mL, about 20 mg/mL, about 25 mg/mL, about 30 mg/mL, about 35 mg/mL, about 40 mg/mL, about 45 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, or about 100 mg/mL. In some embodiments, a pharmaceutical composition can comprise an antibody or antibody conjugate disclosed herein in a concentration of about 1 mg/mL. In some embodiments, a pharmaceutical composition can comprise an antibody or antibody conjugate disclosed herein in a concentration of about 10 mg/mL. In some embodiments, a pharmaceutical composition can comprise an antibody or antibody conjugate disclosed herein in a concentration of about 20 mg/mL.

In some embodiments, the projected volume per injection is from about 0.1 mL to about 1.5 mL, from about 0.1 mL to about 0.5 mL, from about 0.5 mL to about 1 mL, or from about 1 mL to about 1.5 mL. In some embodiments, the projected volume per injection is from about 0.1 mL to about 0.5 mL. In some embodiments, the projected volume per injection is from about 0.5 mL to about 1 mL. In some embodiments, the projected volume per injection is about 0.1 mL, about 0.2 mL, about 0.3 mL, about 0.4 mL, about 0.5 mL, about 0.6 mL, about 0.7 mL, about 0.8 mL, about 0.9 mL, about 1.0 mL, about 1.1 mL, about 1.2 mL, about 1.3 mL, about 1.4 mL, or about 1.5 mL. In some embodiments, the projected volume per injection is about 0.1 mL. In some embodiments, the projected volume per injection is about 0.2 mL. In some embodiments, the projected volume per injection is about 0.3 mL. In some embodiments, the projected volume per injection is about 0.4 mL. In some embodiments, the projected volume per injection is about 0.5 mL. In some embodiments, the projected volume per injection is about 0.6 mL. In some embodiments, the projected volume per injection is about 0.7 mL. In some embodiments, the projected volume per injection is about 0.8 mL. In some embodiments, the projected volume per injection is about 0.9 mL. In some embodiments, the projected volume per injection is about 1 mL.

In some embodiments, a pharmaceutical composition comprising an antibody or antibody conjugate disclosed herein can be administered once a week. In some embodiments, a pharmaceutical composition comprising an antibody or antibody conjugate disclosed herein can be administered once every two weeks. In some embodiments, a pharmaceutical composition comprising an antibody or antibody conjugate disclosed herein can be administered once a month. In some embodiments, a pharmaceutical composition comprising an antibody or antibody conjugate disclosed herein can be administered once every 6 weeks. In some embodiments, a pharmaceutical composition comprising an antibody or antibody conjugate disclosed herein can be administered once every two months.

In some embodiments, a pharmaceutical composition comprising an antibody or antibody conjugate disclosed herein can be administered subcutaneously. In some embodiments, a pharmaceutical composition comprising an antibody or antibody conjugate disclosed herein can be administered subcutaneously using an injectable pen.

### Co-Administration

The pharmaceutical compositions comprising an antibody or antibody conjugate of the disclosure may be used alone, or in combination with one or more other therapeutic agents. The disclosure provides any of the uses, methods or compositions as defined herein wherein the pharmaceutical compositions of the disclosure comprising an antibody or antibody conjugate disclosed herein, is used in combination with one or more other therapeutic agent discussed herein.

The administration of two or more compounds or pharmaceutical compositions “in combination” means that all of the compounds or pharmaceutical compositions are administered closely enough in time to affect treatment of the subject. The two or more compounds or pharmaceutical compositions may be administered simultaneously or sequentially, via the same or different routes of administration, on same or different administration schedules and with or without specific time limits depending on the treatment regimen. Additionally, simultaneous administration may be carried out by mixing the compounds or pharmaceutical compositions prior to administration or by administering the compounds or pharmaceutical compositions at the same point in time but as separate dosage forms at the same or different site of administration. Examples of “in combination” include, but are not limited to, “concurrent administration,” “co-administration,” “simultaneous administration,” “sequential administration” and “administered simultaneously”.

A pharmaceutical compositions comprising an antibody or antibody conjugate of the disclosure and the one or more other therapeutic agents may be administered as a fixed or non-fixed combination of the active ingredients. The term “fixed combination” means a pharmaceutical compositions of the disclosure comprising an antibody or antibody conjugate disclosed herein, and the one or more therapeutic agents, are both administered to a subject simultaneously in a single composition or dosage. The term “non-fixed combination” means that a pharmaceutical compositions and the one or more therapeutic agents are formulated as separate compositions or dosages such that they may be administered to a subject in need thereof simultaneously or at different times with variable intervening time limits, wherein such administration provides effective levels of the two or more compounds or active ingredients in the body of the subject.

In one embodiment, the pharmaceutical compositions of this disclosure are administered in combination with the specifically named agents including the pharmaceutically acceptable salts of the specifically named agents and the pharmaceutically acceptable solvates of said agents and salts. In another embodiment, the disclosure provides methods of treatment that include administering pharmaceutical compositions of the present disclosure in combination with one or more other pharmaceutical agents, wherein the one or more other pharmaceutical agents may be selected from the agents discussed herein.

The anti-GIPR antibodies and anti-GIPR antibody conjugates of the invention can be used alone, or in combination with one or more other therapeutic agents. In some embodiments, an anti-GIPR antibody of the disclosure is administered with a GLP-1R agonist. In some embodiments, an anti-GIPR antibody of the disclosure is administered with a dual agonist of GLP-1R and GCGR. In some embodiments, the GLP-1R agonist or the dual agonist of GLP-1R and GCGR is a small molecule or a polypeptide.

In some embodiments, the GLP-1R agonists include liraglutide, albiglutide, exenatide, lixisenatide, dulaglutide, semaglutide, danuglipron, orforglipron, lotiglipron, PF-06954522, HM15211, LY3298176, Medi-0382, NN-9924, TTP-054, TTP-273, efpeglenatide, CT-996, ECC5004, XW004, XW014, MDR-001, ZT002, KN-056, GL0034, GSBR-1290, noiiglutide, RGT-075, TTP-273, HRS-7535, GMA-105, TG103, GZR-18, GX-G6, ecnoglutide, PB-119, QLG2065, beinaglutide, those described in WO2018109607, those described in WO2019239319 (PCT/IB2019/054867 filed Jun. 11, 2019), and those described in WO2019239371 (PCT/IB2019/054961 filed Jun. 13, 2019). In some embodiments, the GLP-1R agonist is Liraglutide, Exenatide, Albiglutide, Dulaglutide and Semaglutide. In some embodiments, the GLP1R agonist or dual agonist of GLP-1R and GCGR is administered orally, intramuscularly, subcutaneously or intravenously. The invention provides any of the uses, methods or compositions as defined herein wherein an anti-GIPR antibody or antibody conjugates of the invention is used in combination with one or more other therapeutic agent discussed herein.

In one embodiment, the pharmaceutical compositions comprising an antibody or antibody conjugate of this disclosure are administered with an anti-diabetic agent including but not limited to a biguanide (e.g., metformin), a sulfonylurea (e.g., tolbutamide, glibenclamide, gliclazide, chlorpropamide, tolazamide, acetohexamide, glyclopyramide, glimepiride, or glipizide), a thiazolidinedione (e.g., pioglitazone, rosiglitazone, or lobeglitazone), a glitazar (e.g., saroglitazar, aleglitazar, muraglitazar or tesaglitazar), a meglitinide (e.g., nateglinide, repaglinide), a dipeptidyl peptidase 4 (DPP-4) inhibitor (e.g., sitagliptin, vildagliptin, saxagliptin, linagliptin, gemigliptin, anagliptin, teneligliptin, alogliptin, trelagliptin, dutogliptin, or omarigliptin), a glitazone (e.g., pioglitazone, rosiglitazone, balaglitazone, rivoglitazone, or lobeglitazone), a sodium-glucose linked transporter 2 (SGLT2) inhibitor (e.g., empagliflozin, canagliflozin, dapagliflozin, ipragliflozin, Ipragliflozin, tofogliflozin, sergliflozin etabonate, remogliflozin etabonate, or ertugliflozin), an SGLTL1 inhibitor, a GPR40 agonist (FFAR1/FFA1 agonist, e.g. fasiglifam), GIP and analogues thereof, an alpha glucosidase inhibitor (e.g. voglibose, acarbose, or miglitol), or an insulin or an insulin analogue, including the pharmaceutically acceptable salts of the specifically named agents and the pharmaceutically acceptable solvates of said agents and salts.

### Kits

Another aspect of the invention provides kits comprising the anti-GIPR antibody or the anti-GIPR antibody conjugates of the invention or pharmaceutical compositions thereof. A kit may include, in addition to the antibody or antibody conjugates of the invention or pharmaceutical composition thereof, diagnostic or therapeutic agents. A kit may also include instructions for use in a diagnostic or therapeutic method. In some embodiments, the kit includes the antibody or a pharmaceutical composition thereof and a diagnostic agent. In other embodiments, the kit includes the antibody or a pharmaceutical composition thereof and one or more therapeutic agents.

In yet another embodiment, the invention comprises kits that are suitable for use in performing the methods of treatment described herein. In one embodiment, the kit contains a first dosage form comprising one or more of the anti-GIPR antibodies or anti-GIPR antibody conjugates of the invention in quantities sufficient to carry out the methods of the invention. In another embodiment, the kit comprises one or more antibodies of the invention in quantities sufficient to carry out the methods of the invention and at least a first container for a first dosage and a second container for a second dosage.

### EXAMPLES

The following examples of specific aspects for carrying out the present invention are offered for illustrative purposes only and are not intended to limit the scope of the present invention in any way.

The foregoing description and following Examples detail certain specific embodiments of the disclosure and describes the best mode contemplated by the inventors. It will be appreciated, however, that no matter how detailed the foregoing may appear in text, the disclosure may be practiced in many ways and the disclosure should be construed in accordance with the appended claims and any equivalents thereof.

Although the disclosed teachings have been described with reference to various applications, methods, kits, and compositions, it will be appreciated that various changes and modifications can be made without departing from the teachings herein and the claimed disclosure below. The following examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

### Example 1: Kinetic Evaluation of Anti-GIPR Antibody Using Surface Plasmon Resonance (SPR)

Human GIPR ECD (amino acid sequence of SEQ ID NO: 33) was prepared. An anti-human Fc sensor chip was prepared by amine coupling of anti-human IgG antibody (catalog number BR-1008-39, Cytiva) to all flow cells of a carboxymethylated dextran coated sensor chip (CM5) (catalog number BR100530, Cytiva) according to the manufacturer's protocols. The flow cells were activated by injecting a 1:1 mixture of 400 mM 1-Ethyl-3-(3-dimethylaminopropyl) carbodiimide hydrochloride (EDC) and 100 mM N-hydroxysuccinimide (NHS) for 7 minutes at a flow rate of 10 μL/minute. Anti-human IgG antibody was diluted to ˜12.5 μg/mL in 10 mM sodium acetate pH 5.0 and injected over all flow cells for 7 minutes at 10 μL/minute. All flow cells were blocked with 1M ethanolamine-HCl (ETH) for 7 minutes at 10 μL/minute. Final immobilization levels of the capture antibody were approximately 12,000 resonance units (RU). The running buffer for immobilization and kinetics was HBS-EP+ (10 mM 4-(2-hydroxyethyl)-1-piperazineethanesulfonic acid (HEPES) pH 7.4, 150 mM sodium chloride, 3 mM ethylenediaminetetraacetic acid (EDTA), 0.05% (v/v) Tween-20).

To characterize the kinetic binding affinity of antibodies to human GIPR ECD, antibodies were diluted to ˜0.5 μg/mL in HBS-EP+ and captured by the anti-human IgG immobilized on the flow cells for ˜60 seconds at a flow rate of 10 μL/minute. Flow cell 1 was used as a reference surface. After antibody capture, the flow rate was increased to 50 μL/minute and buffer or human GIPR ECD proteins ranging in concentration from 900 to 3.7 nM in HBS-EP+ were injected over all flow cells for a 60 second association period and then allowed to dissociate for 420 seconds. HBS-EP+ buffer cycles collected for each captured antibody were used for double-referencing (Myszka D G. J. Mol. Recognition 1999; 12(5):279-84). At the end of each cycle, the entire anti-IgG surface was regenerated with 3 60 second pulses of 3 M MgCl₂. Kinetic assays were conducted at 37° C. using a collection rate of 10 Hertz (Hz) on a BIAcore™ 8K instrument (Cytiva). Rate constants and affinities were determined by fitting the data to a 1:1 binding model in BIAcore™ Insight Evaluation software version 3.0.12 (GE Healthcare).

Kinetic binding affinities were determined for human GIPR ECD for anti-GIPR antibodies GIPR-0107 and GIPR-2767 and benchmark antibody 2G10 by first binding the antibody to immobilized anti-human IgG. 2G10 is the fully human monoclonal anti-GIPR antagonist antibody portion of AMG 133 (maridebart cafraglutide), a bispecific molecule including two GLP-1 analogue agonist peptides via amino acid linkers. After antibody capture, dilutions of the human GIPR ECD proteins were flowed over the sensor chip surface and the association rate constant (ka), dissociation rate constant (kd), and K_{D} values were determined for binding to human GIPR ECD (Table 5). In this SPR analysis the affinity (K_{D}) of antibodies GIPR-0107 and GIPR-2767 to human GIPR ECD was 13.78, 1.91, and 19.2, respectively.

TABLE 5
Binding Affinities by SPR Against human GIPR ECD

| Ligand | | | | |
|---|---|---|---|---|
| Number | Analyte Name | ka (1/Ms) | kd (1/s) | K_{D} (nM) |
| GIPR-0107 | Human GIPR | 3.63E+05 | 5.01E−03 | 13.78 ± 0.14 |
| | ECD | | | |
| GIPR-2767 | Human GIPR | 6.84E+05 | 1.30E−03 | 1.91 ± 0.14 |
| | ECD | | | |
| 2G10 | Human GIPR | 6.34E+05 | 1.22E−02 | 19.2 ± 1.6 |
| | ECD | | | |

### Example 2. Functional In Vitro GIPR Antagonist Potency Assay

The functional in vitro antagonist potency for anti-GIPR mAbs was determined by monitoring intracellular cyclic adenosine monophosphate (cAMP) levels in Chinese hamster ovary (CHO)-K1 cells stably expressing the human GIPR (hGIPR). Following agonist activation, hGIPR associates with the G-protein complex causing the Gas subunit to exchange bound Guanosine diphosphate (GDP) for guanosine triphosphate (GTP), followed by dissociation of the Gas-GTP complex. The activated Gas subunit can couple to downstream effectors to regulate the levels of second messengers or cAMP within the cell. Thereby, determination of intracellular cAMP levels allows for pharmacological characterization. Intracellular cAMP levels were quantified using a homogenous assay utilizing the Homogeneous Time Resolved Fluorescence (HTRF) technology from Perkin Elmer. The method was a competitive immunoassay between native cAMP produced by the cells and cAMP labelled with the acceptor dye, d2. The two entities competed for binding to a monoclonal anti-cAMP antibody labeled with cryptate. The specific signal was inversely proportional to the concentration of cAMP in the cells.

Anti-GIPR mAbs were diluted to a concentration of 3 mM in cAMP assay base buffer consisting of HBSS (Lonza Cat No. 10-527) containing 20 mM HEPES (Lonza, Cat No. 17-737E). A 14-point dilution series using 1 in 2-fold serial dilutions was created in cAMP assay base buffer with a top concentration of 3 mM. The serially diluted compound was spotted into a 384-well assay plate at 5 mL/well with duplicate/triplicate points at a 3× final assay concentration (FAC). The final compound concentration range in the assay was 1 μM to 100 pM.

Frozen assay-ready vials of CHO-K1 cells stably expressing the Gs-coupled human GIPR receptor (Eurofins, DiscoverX, Cat No. 95-0146C2) were thawed and resuspended in 2× cAMP assay buffer consisting of cAMP assay base buffer+0.2% bovine serum albumin (BSA, Sigma, Cat No. A7979), and 400 μM IBMX (Sigma, Cat No. 15879) at a density of 0.4×10⁶ cells/mL. Cells were added to assay plate (5 μL/well of 0.4×10⁶ cells/mL stock for 2,000 cells/well final) and incubated at 37° C. for 30 minutes. Following the 30-minute cell and compound incubation, hGIPR agonist human glucose-dependent insulinotropic polypeptide (hGIP, full length, Sigma Cat No. G2269) in 1× cAMP assay buffer was added to the assay plate (5 μL/well) at 4.5 nM (final concentration of 1.5 nM) and incubated for another 30 minutes at 37° C., after which intracellular cAMP levels were quantified with Cisbio cAMP-GS Dynamic kit (#62AM4PEC) with dilution of 20× of D2 and Eu in lysis buffer, mixed 1:1 by volume, and added 15 ul/well into plate, with a quick spin. The plate was covered with aluminum foil film and kept on a shaker at room temperature for 1 hr. The plate was then read on an Envision plate reader. Data were analyzed using the ratio of fluorescence intensity at 620 and 665 nm for each well. Data expressed as ratio were normalized and reported as percentage of effect (Zero percentage effect was defined as ratio generated from no hGIP stimulation, 100% effect was defined as data ratio generated from 1.5 nM hGIP stimulation). The concentration and % effect values for each compound were plotted by GraphPad Prism using a four-parameter non-linear fit, and the concentration required for 50% inhibition (IC₅₀) was determined. The Human GIPR-CHO cAMP antagonist activity of anti-GIPR antibodies is shown in Table 6. The IC50 for the antagonist activity in the human GIPR-CHO cAMP assay is 14.4 nM, 28.0 nM, and 30.4 nM for GIPR-107, GIPR-2767, and 2G10, respectively.

TABLE 6
Human GIPR-CHO cAMP antagonist activity
of anti-GIPR antibodies

| | Human GIPR-CHO cAMP Antagonist |
|---|---|
| Antibody | Activity IC50 (nM) |
| GIPR-0107 | 14.4 ± 4.8 (N = 4) |
| GIPR-2767 | 28.0 ± 2.6 (N = 3) |
| 2G10 | 30.4 ± 6.3 (N = 7) |

### Example 3: Biophysical Characterization of Humanized Anti-GIPR Antibodies

Thermal stability: Differential scanning calorimetry was used to determine the stability of the anti-GIPR antibodies GIPR-0107 and GIPR-2767. For this analysis, proteins were diluted in 20 mM histidine, 8.5% sucrose, 0.05 mg/mL EDTA pH 5.8 to 0.3 mg/mL in a volume of 400 μL. Buffer blank was used in the reference cell. Samples were dispensed into the sample tray of a MicroCal PEAQ DSC with Autosampler (Malvern Panalytical). Samples were equilibrated for 5 minutes at 10° C. and then scanned up to 110° C. at a rate of 100° C. per hour. Raw data was baseline corrected and the protein concentration was normalized. The software was used to fit the data to an MN2-State Model with an appropriate number of transitions. Table 7 below shows the melting temperatures (Tₘ1-Tₘ3 and FAB) of the molecule in His/Suc buffer. These molecules showed good stability, with the first transition in the CH2 domain (Tₘ1) of greater than 65° C.

TABLE 7
Thermal stability of anti-GIPR antibodies determined
by differential scanning calorimetry.

| | | | | | apparent |
|---|---|---|---|---|---|
| anti- | | | | | Tm of |
| body | formulation | Tm1(° C.) | Tm2(° C.) | Tm3(° C.) | Fab(° C.) |
| GIPR- | His/Suc/EDTA, | 68.33 ± | 84.93 ± | 88.82 ± | 88.1 |
| 0107 | pH 5.8 | 0.03 | 0.05 | 0.02 | |
| GIPR- | His/Suc/EDTA, | 68.82 ± | 80.93 ± | 82.87 ± | 82.4 |
| 2767 | pH 5.8 | 0.01 | 0.05 | 0.02 | |

Forced Degradation: Anti-GIPR antibodies GIPR-0107 and GIPR-2767 were prepared for a 4 week forced degradation study at 40° C. by extensive dialysis into 20 mM Tris pH 7.5, 20 mM histidine pH 5.8, and 20 mM glutamic acid pH 4.5 using membrane cassette devices 10K MWCO (Thermo Scientific). The protein concentration was adjusted to approximately 5 mg/mL and measured using the Stunner Instrument (Unchained labs). Samples were transferred to a screw cap vial and stored at 40° C. At designated time points (T0, and 2 weeks), aliquots were removed for iCE and stored at −20° C. until thawed for analysis in Imaged capillary isoelectric focusing (iCE) and SPR analysis.

Imaged capillary isoelectric focusing (iCE) was used to detect the charge-based heterogeneity of the forced degradation samples. iCE is used to separate protein charge species under high voltage and detection at absorbance 280 nm. Carrier ampholytes produce a pH gradient and proteins migrate until their net charge is zero. Electropherograms were analyzed to determine pl values and peak areas for acidic, main, and basic species. A protein Simple iCE3 instrument with PrinCE Autosampler was used to analyze samples. Proteins were diluted to 2 mg/mL in water. The sample diluent contained 0.01 mg/mL pl marker 7.55, 0.01 mg/mL pl marker 10.1, 1.0% Pharmalyte pH 5-8, 3.0% Pharmalyte pH 8-10.5, 0.25% methyl cellulose, 2.0 M urea, 4.25 mM arginine. The samples contained 15 μL protein at 2 mg/mL and 85 μL sample diluent. Samples were focused for 1 minute at 1500 Volts and then 9 min at 3000 Volts. Analysis of the samples Tis, His and Glu buffers showed typical increases in the acidic and basic species (Table 8).

TABLE 8
iCE Analysis of forced degradation samples
of optimized anti-GIPR antibodies.

| | | | % acidic | | % basic |
|---|---|---|---|---|---|
| | 40° C. | iCE pl | species | % main | species |
| Antibody | | | | | |
| GIPR-0107 | | | | | |
| Tris, pH 7.5 | T0W | 8.7 | 17.8 | 71.1 | 11.1 |
| | T2W | 8.7 | 35.8 | 53.6 | 10.5 |
| His, pH 5.8 | T0W | 8.7 | 17.4 | 71.4 | 11.2 |
| | T2W | 8.7 | 29.3 | 58.3 | 12.4 |
| Glu, pH 4.5 | T0W | 8.7 | 17.5 | 70.0 | 12.5 |
| | T2W | 8.7 | 31.7 | 55.5 | 12.8 |
| Antibody | | | | | |
| GIPR-2767 | | | | | |
| Tris, pH 7.5 | T0W | 9.2 | 24.7 | 69.7 | 5.6 |
| | T2W | 9.2 | 37.6 | 56.0 | 6.5 |
| His, pH 5.8 | T0W | 9.2 | 21.9 | 73.3 | 4.8 |
| | T2W | 9.2 | 29.1 | 65.1 | 5.8 |
| Glu, pH 4.5 | T0W | 9.2 | 22.7 | 72.0 | 5.3 |
| | T2W | 9.2 | 34.6 | 57.2 | 8.3 |

High Concentration Stability: Anti-GIPR antibodies GIPR-0107 and GIPR-2767 were dialyzed extensively into His/Suc (20 mM histidine, 8.5% sucrose, 0.005% EDTA, at pH 5.8). After dialysis, the samples were concentrated to 150 mg/mL using spin filter concentrator and store at 25° C. At time zero (T0), two weeks (T2W), four weeks (T4W) and six weeks (T6W) samples were aliquoted for real time aSEC analysis, with iCE and CGE analysis at TO, T2W and T6W performed at the end of study.

The aggregation state of the high concentration samples was determined by aSEC. Samples were injected onto a YMC-Pack Diol-200 column (300×8.0 mm, pore size 200 Å) connected to the Agilent 1260 HPLC system (Agilent Technologies, Santa Clara, CA) with 20 mM sodium phosphate, 400 mM arginine at pH 7.2 or 20 mM sodium phosphate, 400 mM NaCl at pH 7.2 as the mobile phase for GIPR-2767 and GIPR-0107 respectively. An isocratic program at a flow rate of 0.75 mL/minute for 20 minutes was applied to the aSEC column for sample elution and the data was analyzed using the Agilent OpenLAB Data Analysis software to integrate and quantify peak area of aggregate, protein of interest and low molecular weight species. Samples at 25° C. showed good high concentration stability at all time points with little increase in HMMS or LMMS (<5% of the total) (Table 9).

TABLE 9
aSEC analysis of 25° C. high concentration
samples of optimized anti-GIPR antibodies.
antibody GIPR-0107

| time | % HMMS | % LMMS |
|---|---|---|
| T0 | 0.61 | 0.02 |
| T2W | 0.70 | 0.05 |
| T4W | 0.72 | 0.07 |
| T6W | 0.77 | 0.12 |

antibody GIPR-2767

| | % acidic | |
|---|---|---|
| time | species | % main |
| T0 | 0.13 | 0.03 |
| T2W | 0.23 | 0.08 |
| T4W | 0.28 | 0.06 |
| T6W | 0.37 | 0.10 |

iCE was used to detect the charge-based heterogeneity of the high concentration samples using a method similar to the method described above for the forced degradation study. Minimal change in the % acidic and the % basic species was seen in antibodies stored at 25° C. in His/Suc buffer over the 6 week study (Table 10).

TABLE 10
iCE analysis of 25° C. high concentration
samples of anti-GIPR antibodies.

| | | | |
|---|---|---|---|
| | % acidic | | |
| buffer/time | species | % main | % basic species |

antibody GIPR-0107

| | | | |
|---|---|---|---|
| His/Suc T0 | 19.4 | 68.3 | 12.3 |
| His/Suc T2W | 20.7 | 67.5 | 11.8 |
| His/Suc T6W | 23.6 | 64.2 | 12.3 |

antibody GIPR-2767

| | | | |
|---|---|---|---|
| His/Suc T0 | 21.3 | 73.2 | 5.5 |
| His/Suc T2W | 22.7 | 70 | 7.3 |
| His/Suc T6W | 25.8 | 68 | 6.2 |

CGE analysis was conducted to determine the fragmentation of the high concentration samples of anti-GIPR antibodies GIPR-0107 and GIPR-2767. Detection and quantitation of sample impurities was performed by CGE using a LabChip GXII instrument from Perkin Elmer. The protein express chip and reagents were purchased from the manufacturer. Proteins were diluted to 1 mg/mL in water. Samples were run under reducing conditions by adding OTT to the commercial sample buffer. Samples were prepared by mixing 5 μL protein with 35 μl sample buffer in a 96 well plate. The plate was covered with foil and samples were denatured for 10 minutes at 7000C on a heat block. The plate was briefly centrifuged and 70 μL of water was added to the samples before loading on the instrument. A virtual gel and electropherogram was generated for each sample and analyzed using LabChip GXII software. The quantification of the % fragmentation and % HMMS demonstrated good high concentration stability for antibodies GIPR-0107 and GIPR-2767 the His/Suc at 25° C. (Table 11).

TABLE 11
CGE analysis of 25° C. high concentration
samples of anti-GIPR antibodies.

| | | | % non HLC | |
|---|---|---|---|---|
| GIPR-0107 | | | % fragment | % other |
| buffer/time | sum of % HLC | % non HLC | (LMMS) | (HMMS) |
| His/Suc, T0 | 97.0 | 3.0 | 2.7 | 0.3 |
| His/Suc, T2 W | 97.2 | 2.8 | 2.5 | 0.3 |
| His/Suc, T6 W | 96.6 | 3.4 | 3.1 | 0.3 |
| | | | % non HLC | |
| GIPR-2767 | | | % fragment | % other |
| buffer/time | sum of % HLC | % non HLC | (LMMS) | (HMMS) |
| His/Suc, T0 | 99.0 | 1.0 | 0.8 | 0.2 |
| His/Suc, T2 W | 98.7 | 1.3 | 1.0 | 0.3 |
| His/Suc, T6 W | 98.6 | 1.4 | 1.1 | 0.3 |

Viscosity: Viscosity of antibodies GIPR-0107 and GIPR-2767 were measured using the following DLS bead-based method. Proteins were extensively dialyzed against 20 mM histidine, 85 mg/ml sucrose, 0.05 mg/ml EDTA pH 5.8 using membrane cassette devices 10K MWCO (Thermo Scientific). Proteins were harvested from dialysis and filtered using a 0.2 micron syringe filter. Proteins were concentrated using Vivaspin centrifugal concentrators 10K MWCO (GE Healthcare). Sample aliquots (12 μL) were removed from the concentrator retentate as the protein volume was reduced and the protein concentration increased. 300 nm beads (Nanosphere, Thermo Scientific) were added to the protein samples and buffer blank. The beads were diluted 1:10 in 20 mM histidine, 85 mg/ml sucrose, 0.05 mg/ml EDTA pH 5.8 and 0.75 μl diluted beads were spiked into the protein sample. The protein/bead and buffer/bead samples were mixed by gently vortexing. 8 μl sample was transferred to 1536 well plate (SensoPlate, glass bottom, Greiner Bio-One) for analysis by DLS. The plate was sealed with optically clear tape and centrifuged at 2000 RPM for 2 minutes to remove bubbles.

The dynamic light scattering measurements were made using a DynaPro Plate Reader (Wyatt Technology, Santa Barbara, CA). Samples were incubated at 25° C. and measured with 15 consecutive 25 second acquisitions. Radius of the bead was averaged for data acquisitions that had acceptable decay curves. The viscosity was calculated based on the Stokes-Einstein equation. Sample viscosity was calculated as the measured apparent radius divided by the nominal bead radius times 0.893 cP, the viscosity of water at 25° C.

High concentration formulation is a desirable property for antibodies, allowing higher and less frequent dosing. Both anti-GIPR antibodies GIPR-0107 and GIPR-2767 showed good viscosity profiles with their viscosity not reaching 20 cP until >150 and >178 mg/mL respectively.

TABLE 12
Viscosity measurements of anti-GIPR antibodies
GIPR-0107 and GIPR-2767 using DLS method.

| GIPR-0107 | | GIPR-2767 | |
|---|---|---|---|
| Concentration | | Concentration | |
| (mg/ml) | Viscosity (cP) | (mg/ml) | Viscosity (cP) |
| 0.0 | 0.9 | 0.0 | 0.9 |
| 50.0 | 2.4 | 50.0 | 2.2 |
| 75.0 | 3.5 | 75.0 | 2.5 |
| 100.0 | 4.6 | 100.0 | 3.7 |
| 125.0 | 7.8 | 125.0 | 5.3 |
| 150.0 | 12.8 | 150.0 | 9.8 |
| 180.8 | 25.2 | 178.4 | 17.4 |

### Example 4: Immunogenicity Risk and T-Cell Epitope Prediction of Anti-GIPR Antibodies

The immunogenicity of the amino acids of anti-GIPR antibodies GIPR-0107 and GIPR-2767 were analyzed using the two methods described below to identify T-cell epitopes. Any sequences flagged using the described protocols was considered an epitope. Sequences were analyzed using EpiMatrix analysis in the ISPRI software package (ISPRI v 1.8.0., EpiVax Inc., Providence RI; Schafer et al., 1998). The raw results ranked the likelihood of binding of each 9-mer amino acid peptide against 8 different HLA types. 9-mer amino acid peptides that were identified as natural T regulatory cell epitopes (T-regitopes) that bind different HLA types were excluded as EpiVax hits. Sequences were also analyzed using the MHC-II binding consensus method (Wang et al. 2010; Wang et al, 2008) in IEDB (IEDB MHC-II Binding Predictions, Vita et al., 2015). The raw results ranked the likelihood of binding of each 9-mer amino acid peptide against the same 8 different HLA types. If either method identified a 9-mer amino acid peptide as a predicted epitope it was classified as an epitope. Each epitope was classified as a germline or non-germline epitope. For antibodies, each epitope was further classified on its location within the antibody complementarity determining region (CDR) or non-CDR location. Two non-germline epitopes were identified in the VH and VL sequences of GIPR-0107 (H32: YAINWVRQA (SEQ ID NO: 143), H63: IFGIANYAQ (SEQ ID NO: 144)) and one non-germline epitope was identified in the VH and VL sequences of GIPR-2767 (L88: CQQSYSTPL (SEQ ID NO: 145) (Table 13).

TABLE 13
Immunogenicity analysis of optimized anti-GIPR antibodies.

| | VH | VL | epitopes | | | |
|---|---|---|---|---|---|---|
| | Seq ID | Seq ID | | non- | | non- |
| antibody | NO | NO | all | germline | CDR | germline CDR |
| GIPR-0107 | 1 | 9 | 10 | 2 | 7 | 2 |
| GIPR-2767 | 14 | 22 | 10 | 1 | 7 | 1 |

### Example 5: Self-Interaction and Polyreactivity of Anti-GIPR Antibodies

The anti-GIPR antibodies GIPR-0107 and GIPR-2767 were tested for non-specific binding to DNA and insulin, tested for non-specificity and self-interaction in an AC-SINS assay (affinity-capture self-interaction nanoparticle spectroscopy; Liu et al. (2014) mAbs 6:483-92), and tested for non-specificity in a Human FcRn interaction assay.

For the DNA and Insulin binding Elisa, 384-well ELISA plates (Nunc Maxisorp) were coated overnight at 4° C. with DNA (10 μg/mL) and insulin (5 μg/mL) in PBS pH 7.2. The ELISA, adapted from assays described in Tiller et al., J. Immunol. Methods 329, 112, 2008; U.S. Pat. No. 7,314,622, was carried out on a PerkinElmer Janus liquid handling robot. Wells were washed with water, blocked with 50 μL of Polyreactivity ELISA Buffer (PEB; PBS pH 7.2 containing 0.05% Tween-20, 1 mM EDTA) for 1 hour at room temperature, and rinsed once with 80 μL of water. Test samples at 10 μg/mL (in PBS pH 7.2, 0.05% Tween-20, 1 mM EDTA) were added in quadruplicate to the wells and incubated for 1 h at room temperature. Plates were washed 3 times with 80 μL of water, and 25 μL of 32 ng/ml goat anti-human IgG (Fcγ fragment specific) conjugated to horseradish peroxidase (Jackson ImmunoResearch) in PBS pH 7.2, 0.05% Tween-20, 1 mM EDTA, was added to each well. Plates were incubated for 1 h at room temperature, washed 3 times with 80 μL of water, and 25 μL of TMB substrate (Sigma Aldrich) was added to each well. Reactions were stopped after 6 minutes 45 seconds by adding 25 μL of 0.18 M ortho-phosphoric acid to each well and absorbance was read at 450 nm. DNA- and insulin-binding scores were calculated as the ratio of the ELISA signal of the antibody at 10 μg/mL to the signal of a well containing buffer.

For the AC-Sins assay, proteins had the potential to interact with themselves, particularly at increased concentrations. This self-interaction could lead to viscosity challenges associated with formulation during drug development, as well as increased risk of clearance. (Avery et al. MAbs. 2018; 10(2): 244-255). The AC-SINS assay measures self-interaction and were used to help predict high viscosity and the potential for poor pharmacokinetic properties. The AC-SINS assay was standardized in a 384-well format on a Perkin-Elmer Janus liquid handling robot. 20 nm gold nanoparticles (Ted Pella, Inc., #15705) were coated with a mixture of 80% goat anti-human Fc (Jackson ImmunoResearch Laboratories, Inc. #109-005-098) and 20% non-specific goat polyclonal antibodies (Jackson ImmunoResearch Laboratories, Inc. #005-000-003) that were buffer exchanged into 20 mM sodium acetate pH 4.3 and diluted to 0.4 mg/mL. After one hour incubation at room temperature, sites unoccupied on the gold nanoparticles were blocked with thiolated polyethylene glycol (2 kD). The coated nanoparticles were then concentrated 10-fold using a syringe filter and 10 μL were added to 100 μL of test sample at 0.05 mg/mL in PBS pH 7.2. The coated nanoparticles were incubated with the test sample for 2 hrs in a 96-well polypropylene plate and then transferred to a 384-well polystyrene plate and read on a Tecan spectrophotometer. The absorbance was read from 450-650 nm in 2 nm increments, and a Microsoft Excel macro was used to identify the max absorbance, smooth the data, and fit the data using a second-order polynomial. The smoothed max absorbance of the average blank (PBS buffer alone) was subtracted from the smoothed max absorbance of the sample to determine the AC-SINS score.

For the human FcRn interaction assay, a human FcRn affinity column was prepared as previously described in Koch et al (mAbs 5:576-586, 2013). To determine the human FcRn column retention time, 50 μg of test sample adjusted to pH 5.5 was injected onto a GE High Performance Streptavidin Sepharose column (cat #17-5113-01) coated with in-house expressed and purified biotinylated hFcRn, and a linear pH gradient from pH 5.5 (MES) to 8.8 (Tris) in the presence of 150 mM NaCl was applied for elution of sample. The FcRn column data are reported as a relative retention time where the elution time of sample is subtracted from that of an assay performance control, mAb-A, to normalize variability within assay.

High scores each of these assays been suggested to correlate with low or poor solubility and nonspecific membrane Interactions (Liu et al., mAbs 2014; 6:483-92). Additionally, high scores in each of these assays have been suggested to correlate with poor in vivo properties and faster clearance. (Avery et al., mAbs 2018; 10:244-255). Antibodies GIPR-0107 and GIPR-2767 have low AC-SINS scores, DNA/Insulin scores and FcRn column relative retention times, comparable to those of the negative control (Table 14). These data indicate that these antibodies are at low-risk for polyreactivity, similar to that of the negative control.

TABLE 14
Non-specific binding and self-interaction
of anti-GIPR Antibodies (N = 2).

| | AC-SINS | | | |
|---|---|---|---|---|
| | (wavelength of | | | |
| | maximum | | insulin | hFcRN |
| | absorbance | DNA binding | binding | Relative |
| | relative | normalized | normalized | Retention |
| antibody | to blank, nm) | to blank | to blank | Time |
| GIPR-0107 | 1.5 | 1 | 1 | 1.3 |
| GIPR-2767 | 0 | 3.5 | 2.5 | 0.9 |
| negative | 0 | 1.5 | 1.5 | 0.8 |
| control | | | | |
| positive | 23.5 | 37 | 37.5 | 4.15 |
| control | | | | |

### Example 6. Comparative Data

Two GIPR antagonist mAb/GLP-1 agonist peptide conjugates are in clinical development, AMG133 and GMA106. Table 15 shows a comparison of GIPR-2767, AMG133 (2G10) and GMA106 (L7H6) hGIPR functional antagonist potency, and the types of antagonism. The K_{b} values were obtained using functional cAMP antagonist mode assays (<0.133 μM). The data show that GIPR-2767 is a potent inhibitor of GIPR activation and cAMP production.

TABLE 15
Comparison of hGIPR functional antagonist potency

| Molecule Name | GIPR-2767 | AMG133(2G10) | GMA106(L7H6) |
|---|---|---|---|
| hGIPR functional | K_{b}: 0.147 nM | K_{b}: 0.133 nM | IC₅₀: 0.454 nM |
| antagonist | (competitive) | (competitive) | (non-competitive) |
| potency (type | | | |
| of antagonism) | | | |

### Example 7. Oral Glucose Tolerance Test (OGTT) in Mice of Anti-GIPR Antibodies

To test the efficacy of anti-GIPR antibodies GIPR-0107 and GIPR-2767 in vivo, humanized mice expressing hGIPR were made in which human GIPR is expressed under the control of the endogenous mouse Gipr locus while knocking out the mouse Gipr gene. Human GIPR cDNA-SV40 pA was inserted in-frame with mouse Gipr at the end of exon 2 (1^{st} coding exon) utilizing CRISPR/Cas9 technology. Insertion of human GIPR cDNA and disruption of mouse Gipr was validated by genotyping, and human GIPR mRNA expression was confirmed by quantitative PCR. These hGIPR mice were used to measure the in vivo efficacy of GIPR-0107 and GIPR-2767 using oral glucose tolerance test (OGTT) and weight gain prevention studies.

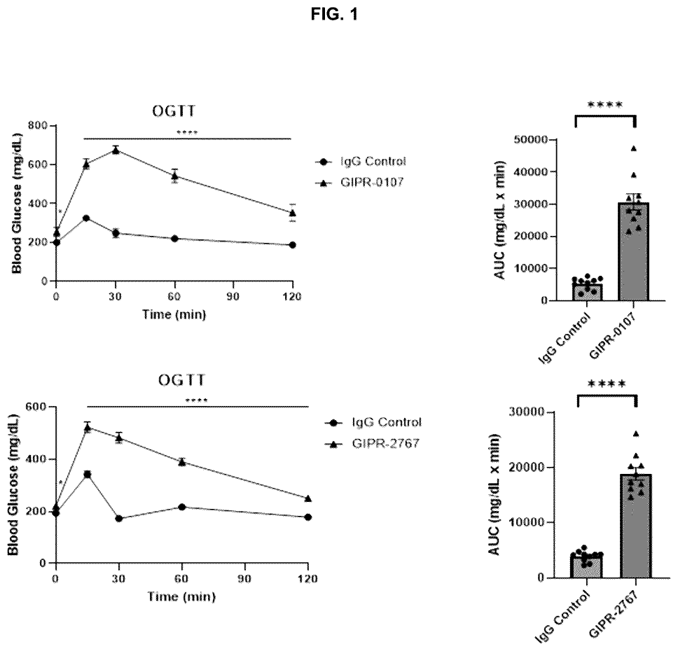

To measure in vivo efficacy of GIPR-0107 and GIPR-2767, an oral glucose tolerance test (OGTT) was performed in diet induced obese (DIO) male hGIPR mice. For GIPR-0107, male hGIPR mice between 14-16 weeks of age were utilized that had been on 60% high fat diet (D12492, Research Diets) for 8 weeks prior to the start of the study. For GIPR-2767, male hGIPR mice between 13-14 weeks of age were utilized that had been on 60% high fat diet (D12492, Research Diets) for 7 weeks prior to the start of the study. Mice were singly housed, maintained on a 12-hour light/12-hour dark cycle, and received food and wr ad libitum. Prior to the start of the study, mice were weighed and randomized into groups based on body weight. The animals were dosed subcutaneously with IgG control, GIPR-0107, or GIPR-2767 at 3 mg/kg in the afternoon before the OGTT. On the morning of the experiment, mice were fasted for 5.5 hours. Before dosing, mice were bled vial tail snip to obtain a basal blood glucose reading by AlphaTRAK handheld glucometer, and at time 0, mice were dosed with 1.5 g/kg glucose by oral gavage. Blood glucose was measured by glucometer at 15 min, 30 min, 60 min, and 120 min after glucose administration. Blood glucose values during the OGTT were used to generate glucose AUC₀₋₁₂₀ min normalized to starting glucose values for each mouse is shown in FIG. 1. A mixed effects model with appropriate fixed, random, correlation, and variance structure is used for the OGTT. If some features are not needed, the model is reduced. A transformation may be needed for violations of the normality assumption. ANOVA with appropriate adjustments for violations of homoscedasticity or normality is used for the OGTT AUC. All residuals were evaluated for meeting the normality assumption with the Shapiro-Wilks Test and Q-Q Plots and the sphericity and homoscedasticity assumption by the residual plots and Bartlett's test. If multiple comparisons are done, the appropriate multiple comparisons method is applied. All analyses performed with R version 4.1.0.

Example 8 demonstrates that GIPR-0107 and GIPR-2767 increase glucose excursion during an OGTT (FIG. 1). During an OGTT, an oral bolus of glucose is given, and blood glucose values are measured over time. After oral glucose administration, GIP is secreted by intestinal cells, which then binds to GIPR and acts to induce insulin secretion by the pancreas into the bloodstream. Insulin secretion results in glucose uptake from circulation by peripheral tissues, resulting in blood glucose lowering. This glucose response curve is shown in FIG. 1 in the IgG control condition. In FIG. 1, Number of animals per group is 10, data are presented as the mean and error bars are SEM. * indicates p<0.05, **** indicates p<0.0001 versus IgG administered animals.

The AUC value is used to quantify the increase in blood glucose over time. Results from FIG. 1 show that anti-GIPR antibodies GIPR-0107 and GIPR-2767 antagonize GIP-induced lowering of blood glucose.

### Example 8. Body Weight Gain Prevention of Mice by Anti-GIPR Antibodies

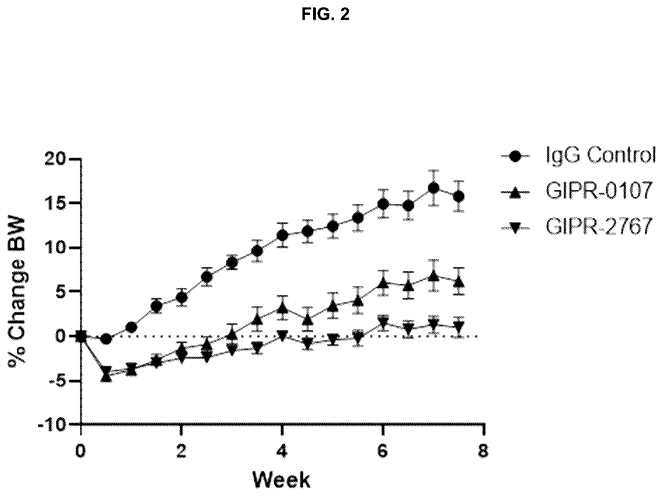

Diet induced obese (DIO) hGIPR mice were also used to assess the in vivo activity of GIPR-0107 and GIPR-2767 following repeat administration of GIPR-0107 and GIPR-2767. Male hGIPR mice between 18-21 weeks of age were utilized which had been on 60% high fat diet (D12492, Research Diets) for 14 weeks prior to the start of the study and were singly housed, maintained on a 12-hour light/12-hour dark cycle, and received food and water ad libitum. Prior to the start of the study, mice were randomized into each group based on body weight. Twice a week, mouse body weight was measured and mice were dosed subcutaneously with IgG control, GIPR-0107, or GIPR-2767 at 1 mg/kg. Mice were continued on 60% high fat diet for the duration of the study. Percent change in body weight at each time point was calculated based on difference from pre-dose weight is summarized in FIG. 2. In FIG. 2, number of animals per group=10; Data are presented as the mean and error bars are SEM.

Example 8 demonstrates that chronic administration of GIPR-0107 and GIPR-2767 reduces body weight gain in a high fat diet weight gain prevention model (FIG. 2).

### Example 9. Oral Glucose Tolerance Test and Body Weight Gain Prevention, Multiple Dose Levels

To measure acute in vivo pharmacology of GIPR-2767 following single dose administration, an oral glucose tolerance test (OGTT) was performed in DIO male hGIPR mice at multiple dose levels. Male hGIPR mice between 22-23 weeks of age were utilized that had been on 60% high fat diet (D12492, Research Diets) for 15 weeks prior to the start of the study. Mice were singly housed, maintained on a 12-hour light/12-hour dark cycle, and received food and water ad libitum. Prior to the start of the study, mice were weighed and randomized into groups based on body weight. The animals were dosed subcutaneously with IgG control at 3 mg/kg or GIPR-2767 at 0.1, 0.3, 1, or 3 mg/kg in the afternoon before the OGTT. On the morning of the experiment, mice were fasted for 5.5 hours. Before dosing, mice were bled via tail snip to obtain a basal blood glucose reading by AlphaTRAK handheld glucometer, and at time 0, mice were dosed with 1.5 g/kg glucose by oral gavage. Blood glucose was measured by glucometer at 15 min, 30 min, 60 min, and 120 min after glucose administration. Blood glucose values during the OGTT were used to generate glucose area under the curve (AUC)₀₋₁₂₀ₘᵢₙ values normalized to starting glucose values for each mouse, shown in Table 16. A mixed effects model with appropriate fixed, random, correlation, and variance structure was used to analyze glucose data from the OGTT. ANOVA with appropriate adjustments for violations of homoscedasticity or normality was used for the OGTT AUC. All residuals were evaluated for meeting the normality assumption with the Shapiro-Wilks Test and Q-Q Plots and the sphericity and homoscedasticity assumption by the residual plots and Bartlett's test. All analyses were performed with R version 4.1.0.

Following the OGTT, mice continued subcutaneous dose administration with either IgG control or GIPR-2767 (0.1, 0.3, 1, or 3 mg/kg) twice weekly for 6 weeks. To determine statistical significance for the body weight endpoint, a generalized least squares linear model with appropriate fixed effects and correlation and variance structure are used for BW outcomes. All residuals were evaluated for meeting the normality assumption with the Shapiro-Wilks Test and Q-Q Plots and the sphericity and homoscedasticity assumption by the residual plots and Bartlett's test. FDR adjustment method is used for selected multiple comparisons. All analyses performed with R version 4.1.0.

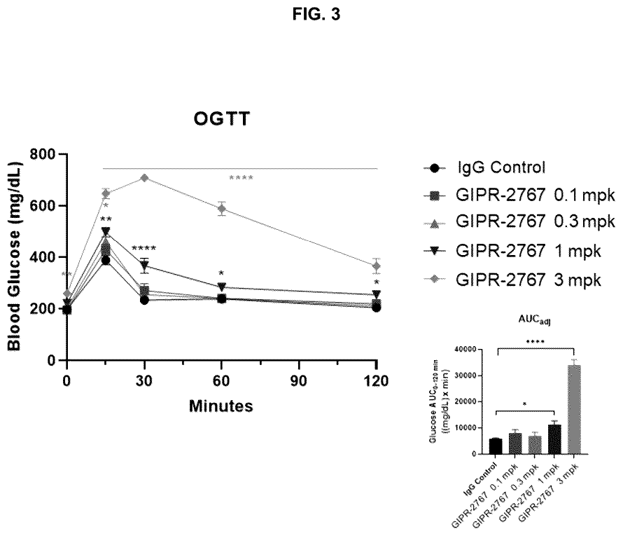

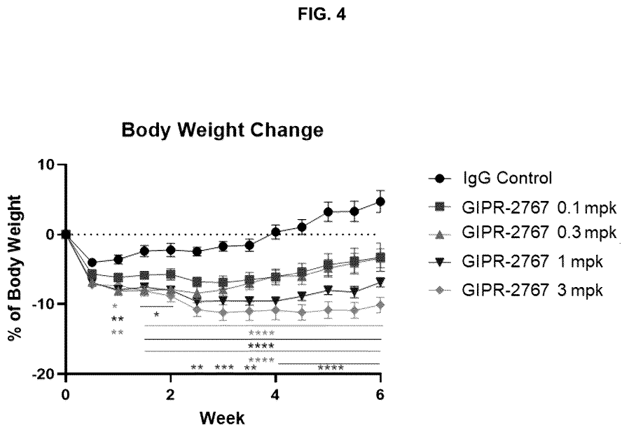

Example 9 demonstrates that GIPR-2767 increases glucose excursion during an OGTT following a single dose of 1 and 3 mg/kg compared to IgG control, and chronic administration of GIPR-2767 reduced body weight gain compared to IgG control treated hGIPR mice on a high fat diet. (Table 16). FIG. 3 depicts the effects of anti-GIPR antibody GIPR-2767 at 0.1 mpk, 0.3 mpk, 1 mpk, and 3 mpk dose levels on blood glucose during an Oral Glucose Tolerance Test (OGTT). FIG. 4 depicts the effect of anti-GIPR antibody GIPR-2767 at 0.1 mpk, 0.3 mpk, 1 mpk, and 3 mpk dose levels body weight shown as percentage change from baseline over a 6 week period.

TABLE 16
Mean Blood Glucose AUC_{0-120 min} During the acute OGTT, multiple dose levels

| | | Glucose | Glucose | | Adjusted |
|---|---|---|---|---|---|
| | Dose | AUC_{0-120 min} | AUC_{0-120 min} | | P Value |
| Compound | (mg/kg) | (mg × min/dL) | 95% CI | n | (Versus IgG) |
| IgG Control | 3 | 5670.9 | 4580.19, 6761.61 | 10 | — |
| GIPR-2767 | 0.1 | 7938.3 | 4577.084, 11299.52 | 10 | 0.2186 |
| GIPR-2767 | 0.3 | 7042 | 3875.205, 10208.8 | 10 | 0.4154 |
| GIPR-2767 | 1 | 11196.3 | 7776.414, 14616.19 | 10 | 0.0105 |
| GIPR-2767 | 3 | 33968.5 | 28806.57, 39130.43 | 10 | 9.99E−07 |

### Example 10. Comparison of GIPR-0107 and GIPR-2767 Against 2G10 in Body Weight Gain Prevention at 1 Mpk

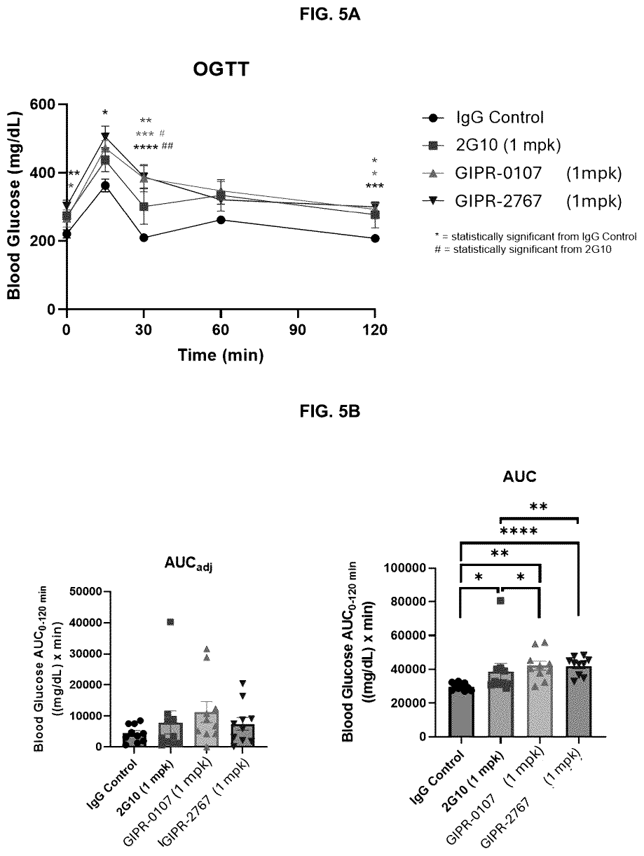

To measure acute in vivo pharmacology of GIPR-0107 and GIPR-2767 compared to 2G10 following single dose administration, an oral glucose tolerance test (OGTT) was performed in DIO male hGIPR mice at 1 mpk using the method described above. FIG. 5A depicts the effect of anti-GIPR antibodies 2G10, GIPR-0107, and GIPR-2767 at 1 mpk on blood glucose during an Oral Glucose Tolerance Test (OGTT). FIG. 5B depicts the effect of anti-GIPR antibodies 2G10, GIPR-0107, and GIPR-2767 at 1 mpk on blood glucose AUC₀₋₁₂₀ min. The baseline glucose levels were higher in some of the groups during the OGTT, so baseline glucose adjusted AUCs were used to correct for baseline glucose differences. In this study, the adjusted AUCs were not significantly different than IgG.

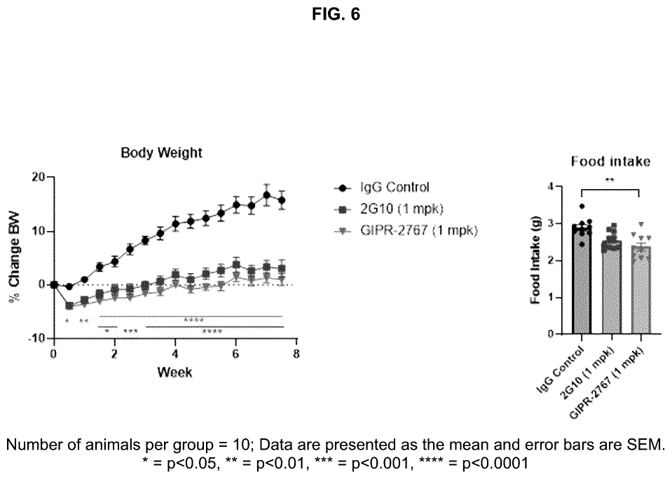

DIO hGIPR mice were also used to assess the in vivo activity of GIPR-2767 following repeat administration. Male hGIPR mice between 18-21 weeks of age were utilized which had been on 60% high fat diet (D12492, Research Diets) for 14 weeks prior to the start of the study and were singly housed, maintained on a 12 hour light/12 hour dark cycle, and received food and water ad libitum. Prior to the start of the study, mice were randomized into each group based on body weight. Twice a week, mouse body weight was measured and mice were dosed subcutaneously with IgG control, 2G10, or GIPR-2767 at 1 mg/kg. Mice were continued on 60% high fat diet for the duration of the study. Percent change in body weight at each time point was calculated based on difference from pre-dose weight, and 24 hour food intake was measured during week 3. A mixed effects model with appropriate fixed, random, correlation, and variance structure was used for longitudinal outcomes. For multiple group outcomes, ANOVA with appropriate adjustments for violations of homoscedasticity or normality was used. All residuals were evaluated for meeting the normality assumption with the Shapiro-Wilks Test and Q-Q Plots and the sphericity and homoscedasticity assumption by the residual plots and Bartlett's test. Tukey's adjustment method is used for all pairwise multiple comparisons. All analyses were performed with R version 4.1.0. As shown in FIG. 6, chronic administration of GIPR-2767 or benchmark antibody 2G10 similarly reduced body weight gain compared to IgG control treated mice. GIPR-2767 administration also reduced acute food intake.

FIG. 6 depicts the effect of chronic GIPR-2767 administration on body weight (% change from baseline) and food intake.

### Example 11. Binding Epitope of Anti-GIPR Antibodies

To identify the binding epitope on GIP of the anti-GIPR antibodies GIPR-0107 and GIPR-2767, the x-ray crystal structure of these antibodies in complex with GIPR-ECD was determined. To solve the x-ray crystal structure of GIPR-0107 FAB in complex with GIPR-ECD, crystals were obtained by sitting-drop vapor-diffusion method from a condition containing 100 mM sodium acetate pH 5, 200 mM sodium chloride, 20% PEG 6000. The crystals had symmetry consistent with monoclinic space group P21 with four copies of complexes in the crystallographic asymmetric unit. A data set to 2.6 Å resolution was collected from a single frozen crystal at IMCA beamline 17-ID at the Argonne National Laboratory (APS). The data were processed and scaled using autoPROC and the structure was solved by molecular replacement with PHASER.

To solve the x-ray crystal structure of GIPR-2767 scfv (GIPR-2767_VH-SSGGGGSGGGGSGGGGS -GIPR-2767_VL; SEQ ID NO: 147) in complex with GIPR-ECD, crystals were obtained by sitting-drop vapor-diffusion method from a condition containing 100 mM HEPES pH 7.5, 1000 mM sodium acetate, 50 mM cadmium sulfate. The crystals had symmetry consistent with orthorhombic space group C222. A data set to 2.1 Å resolution was collected from a single frozen crystal at IMCA beamline 17-ID at the Argonne National Laboratory (APS). The data were processed and scaled using autoPROC and the structure was solved by molecular replacement with PHASER.

The epitope of GIPR-ECD for anti-GIPR antibodies GIPR-0107 and GIPR-2767 along with benchmark antibodies 2G10 (2G10:GIPR-ECD; PDBid: 6dkj, Killion, E. A., et al., Science Translational Medicine, 2018, 10, eaat3392) and GIPG013 (GIPG013:GIPR-ECD; PDBid 4hj0; Ravn, P. et al., The Journal of Biological Chemistry, 2013, 288(27): 19760-19772) and benchmark peptide of GIP peptide (GIP:GIPR; PDBid 7ra3; Sun et al., Proc. Natl. Acad. Sci. USA 2022:119) were determined using a structure-based descriptor (SBD) method. The SBD method utilizes the x-ray crystal structure or cryo-EM structure complex and determines which residues are significantly buried by the complex formation (>20 Angtrom\hat{ }2 buried surface area (BSA)), residues that are participating in hydrogen bonds, residues participating in water mediate hydrogen bonds and residues participating in salt bridges, and residues that are in close contact (<=3.8 angstroms). Residues that fit any of these criteria are determined to be the and epitope. The residues of the antigen and antibody are said to be hydrogen bonded if they include a hydrogen bond donor atom (bound to an electropositive hydrogen) in one molecule located within 3.2 Angstrom of a hydrogen bond acceptor atom having a lone pair of electrons in the other molecule. Residues of the antibody and antigen are said to form a salt bridge if they contain a positively charged atom in one molecule within 4 Angstrom of a negatively charged atom in the other molecule. The per-residue solvent exclusion was determined by calculating the solvent accessible surface area of each residue of the antibody and antigen in complex and subtracting this from the sum of the solvent accessible surface areas of the two components considered individually. The solvent accessible surface area was calculated according to the method of Strake and Rupley (J Mol Biol 79 (2): 351-371, 1973). The pairwise buried surface area was used to estimate the individual contributions of pairs of residues from the antibody and antigen to the overall effect of buried surface area on binding energy. Since buried surface area is not pairwise decomposable, the buried surface area of each residue in the epitope was calculated in the presence of each individual antibody residue in the absence of the rest of the antibody. These individual contributions were then normalized so that the sum of all individual contributions of all antibody residues to the buried surface area of a given epitope residue would equal the total buried surface area of that epitope residue due to the binding of the entire antibody or protein. Finally, a given residue was considered to be part of the epitope if it participates in a hydrogen bond with the antibody or with a water that is also hydrogen bonded to the antibody or if it participates in a salt bridge to a residue in the antibody or if it has a non-zero change in buried surface area due to interaction with the antibody.

The epitope on GIPR-ECD interaction with GIPR-0107, GIPR-2767, 2G10, GIPG013 and GIP, are shown in tables 15, 16, 17, 18, and 19, respectively. The residues in the GIPR-ECD epitope for GIPR-0107 are LEU35, TYR36, TRP39, ASP66, MET67, TYR68, VAL69, PRO85, TYR87, LEU88, TRP90, ARG101, GLN108, TRP109, GLY110, LEU111, ARG113, HIS115, GLU119, and LYS123. The residues in the GIPR-ECD epitope for GIPR-2767 are LEU35, TRP39, ASP66, MET67, TYR68, VAL69, TYR87, TRP90, VAL99, GLN102, GLY104, SER105, ASP106, GLN108, TRP109, GLY110, LEU11, TRP112, ARG113, HIS115, and GLU119. The residues in the GIPR-ECD epitope for 2G10 are THR31, ALA32, GLY33, LEU35, TYR36, GLN37, TRP39, ASP66, MET67, TYR68, TYR87, TRP90, ARG101, ARG113, HIS115, GLU119, ASN120, LYS123, GLU125, LEU128, ASP129, LEU132, and ILE133. The residues in the GIPR-ECD epitope for GIPG013 ALA32, GLY33, LEU35, TYR36, TRP39, GLU40, GLN47, PHE65, MET67, TYR68, TYR87, LEU88, TRP109, HIS115, CYS118, GLU119, and ASN120. The residues in the GIPR-ECD epitope for GIP are GLN30, ALA32, LEU35, TYR36, TRP39, MET67, TYR68, TRP71, TRP90, ARG101, ARG113, HIS115, GLN130, ARG131, LEU134, LEU137, and GLN138. Alignments of the epitopes of GIPR-ECD with GIPR-0107, GIPR-2767, 2G10, GIPG013 and GIP are shown in FIG. 1. GIPR-0107 shows unique epitope residues from benchmark 2G10 and GIPG013 including Val69, Pro85, Gln107, Gly110 and Leu111. GIPR-2767 shows unique epitope residues from benchmark 2G10 and GIPG013 including Val69, Va199, Gln102, Gly104, Ser105, Asp106, Gln108, Gly110, Leu111 and Trp112.

TABLE 17
Epitope sites for GIPR-0107/GIPR-
ECD complex predicted by SBD

| | | | | Close contact |
|---|---|---|---|---|
| | | | | to Antigen |
| | | BSA >20 | Electrostatic | (<=3.8 |
| Type | Site | Angstroms\hat{ }2 | Interactions | Angstroms) |
| LEU | 35 | 23.6 | | |
| TYR | 36 | 87.0 | H-Bond | X |
| TRP | 39 | 27.9 | | X |
| ASP | 66 | | | X |
| MET | 67 | 71.9 | | × |
| TYR | 68 | 147.2 | H-Bond | X |
| VAL | 69 | | | X |
| PRO | 85 | | | X |
| TYR | 87 | | | X |
| LEU | 88 | 25.4 | | |
| TRP | 90 | 30.8 | | X |
| ARG | 101 | | Salt-Bridge | |
| GLN | 108 | 34.9 | H-Bond | X |
| TRP | 109 | 22.3 | H-Bond | X |
| GLY | 110 | | | X |
| LEU | 111 | 105.6 | | X |
| ARG | 113 | 52.3 | H-Bond, Salt-Bridge | X |
| HIS | 115 | 93.3 | H-Bond | X |
| GLU | 119 | 51.6 | | X |
| LYS | 123 | 62.5 | | X |

TABLE 18
Epitope sites for GIPR-27676/GIPR-
ECD complex predicted by SBD

| | | | | Close contact |
|---|---|---|---|---|
| | | | | to Antigen |
| | | BSA >20 | Electrostatic | (<=3.8 |
| Type | Site | Angstrom\hat{ }2 | Interactions | Angstroms) |
| LEU | 35 | | | X |
| TRP | 39 | 23.2 | | X |
| ASP | 66 | 35.1 | H-bond, Water- | X |
| | | | mediated H-Bond | |
| MET | 67 | 39.6 | | X |
| TYR | 68 | 64.3 | | X |
| VAL | 69 | | | X |
| TYR | 87 | 28.8 | H-bond | X |
| TRP | 90 | 20.1 | | |
| VAL | 99 | | | X |
| GLN | 102 | 31.2 | Water-mediated H- | X |
| | | | bond | |
| GLY | 104 | | | X |
| SER | 105 | | H-bond | X |
| ASP | 106 | 40.6 | Salt-Bridge, Water- | X |
| | | | mediated H-bond | |
| GLN | 108 | 44.3 | Water-mediated H- | X |
| | | | bond | |
| TRP | 109 | | Water-mediated H- | |
| | | | bond | |
| GLY | 110 | 21.1 | | X |
| LEU | 111 | 125.3 | H-bond | X |
| TRP | 112 | 44.3 | H-bond, Water- | X |
| | | | mediated H-Bond | |
| ARG | 113 | 67.4 | H-bond, Water- | X |
| | | | mediated H-Bond | |
| HIS | 115 | 55.3 | H-bond, Water- | X |
| | | | mediated H-Bond | |
| GLU | 119 | 21.3 | H-bond | |

TABLE 19
Epitope sites for 2G10/GIPR-ECD complex predicted by SBD

| | | | | Close contact |
|---|---|---|---|---|
| | | | | to Antigen |
| | | BSA >20 | Electrostatic | (<=3.8 |
| Type | Site | Angstroms\hat{ }2 | Interactions | Angstroms) |
| THR | 31 | 52.1 | | X |
| ALA | 32 | 98.6 | H-bond | X |
| GLY | 33 | 98.7 | H-bond | X |
| LEU | 35 | 91.3 | | X |
| TYR | 36 | 89.7 | H-bond | X |
| GLN | 37 | 25.2 | H-bond | X |
| TRP | 39 | 80.5 | | X |
| ASP | 66 | 65.7 | | X |
| MET | 67 | 74.5 | | X |
| TYR | 68 | 91.0 | | X |
| TYR | 87 | | | X |
| TRP | 90 | 56.0 | | |
| ARG | 101 | 39.5 | Water-mediated H- | |
| | | | bond | |
| ARG | 113 | 63.9 | H-bond | X |
| HIS | 115 | 53.8 | H-bond, Water- | X |
| | | | mediated H-bond | |
| GLU | 119 | | Water-mediated H- | X |
| | | | bond | |
| ASN | 120 | | Water-mediated H- | |
| | | | bond | |
| LYS | 123 | 42.7 | H-bond, Salt-Bridge, | X |
| | | | Water-mediated H- | |
| | | | bond | |
| GLU | 125 | 54.6 | H-bond, Salt-Bridge, | X |
| | | | Water-mediated H- | |
| | | | bond | |
| LEU | 128 | 61.0 | | X |
| ASP | 129 | 77.4 | H-bond, Salt-Bridge, | X |
| | | | Water-mediated H- | |
| | | | bond | |
| LEU | 132 | 76.4 | | X |
| ILE | 133 | 34.7 | | X |

TABLE 20
Epitope sites for GIPG013/GIPR-ECD complex predicted by SBD

| | | | | Close contact |
|---|---|---|---|---|
| | | | | to Antigen |
| | | BSA >20 | Electrostatic | (<=3.8 |
| Type | Site | Angstroms\hat{ }2 | Interactions | Angstroms) |
| ALA | 32 | 50.7 | | X |
| GLY | 33 | 21.9 | | X |
| LEU | 35 | 38.6 | | |
| TYR | 36 | 133.6 | H-Bond | X |
| TRP | 39 | 47.6 | | X |
| GLU | 40 | 69.6 | H-Bond | X |
| GLN | 47 | 47.1 | H-Bond | X |
| PHE | 65 | 35.0 | | X |
| MET | 67 | 124.2 | | X |
| TYR | 68 | 139.0 | H-Bond | X |
| TYR | 87 | 28.4 | | X |
| LEU | 88 | 30.1 | | |
| TRP | 109 | 20.2 | | |
| HIS | 115 | 102.5 | H-Bond | X |
| CYS | 118 | | H-Bond | X |
| GLU | 119 | 25.4 | | X |
| ASN | 120 | 50.4 | H-Bond | X |

TABLE 21
Epitope sites for GIP/GIPR complex predicted by SBD

| | | | Electrostatic | Close contact to Antigen |
|---|---|---|---|---|
| Type | Site | BSA | Interactions | (<=3.8 Angstroms) |
| GLN | 30 | 37.4 | | X |
| ALA | 32 | 36.5 | | X |
| LEU | 35 | 43.4 | | |
| TYR | 36 | 41.6 | H-bond | X |
| TRP | 39 | 39.3 | | X |
| MET | 67 | 24.3 | | |
| TYR | 68 | 87.0 | H-bond | X |
| TRP | 71 | | | X |
| TRP | 90 | 52.4 | | X |
| ARG | 101 | | | X |
| ARG | 113 | 53.4 | H-bond | X |
| HIS | 115 | 21.9 | | X |
| GLN | 130 | 25.0 | | |
| ARG | 131 | 70.1 | | X |
| LEU | 134 | 63.3 | | X |
| LEU | 137 | | | X |
| GLN | 138 | 37.2 | H-bond | X |
| TYR | 141 | 39.3 | | X |
| ARG | 183 | 13.9 | H-bond, Salt-bridge | X |
| ILE | 187 | 21.3 | | X |
| ARG | 190 | 40.9 | | X |
| LEU | 194 | 36.9 | | |
| PRO | 197 | 20.6 | | X |
| GLY | 198 | | | X |
| PRO | 199 | 58.9 | | X |
| TYR | 200 | 38.8 | | |
| GLN | 224 | 20.0 | H-bond | X |
| VAL | 227 | 33.0 | | X |
| TYR | 231 | | | X |
| GLU | 288 | 53.9 | H-bond | X |
| ARG | 289 | 86.4 | H-bond, Salt-bridge | X |
| ASN | 290 | 19.3 | H-bond | X |
| GLU | 291 | 20.3 | | |
| TRP | 296 | 50.7 | | X |
| ARG | 300 | 54.0 | H-bond | X |
| ARG | 370 | 30.3 | Salt-bridge | X |
| LEU | 374 | 51.6 | | |
| GLU | 377 | 22.7 | | X |
| ILE | 378 | 50.6 | | X |
| SER | 381 | 11.3 | H-bond | X |

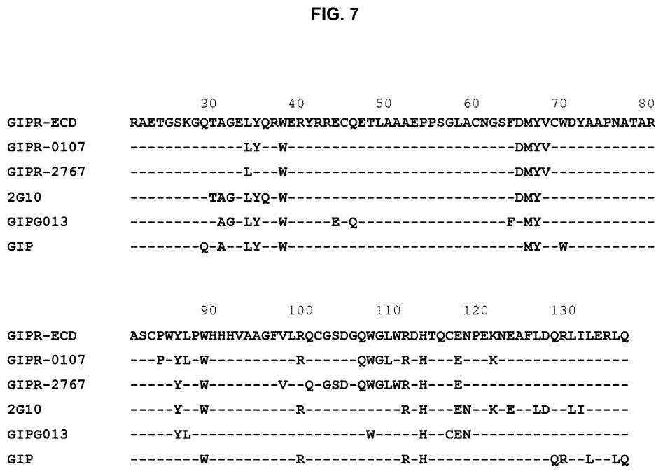

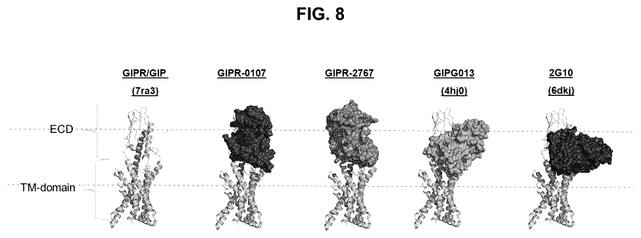

The unique binding site residues between each of GIPR-0107, GIPR-2767, 2G10, and GIPGO13 (FIG. 7) are related to special differences of the binding epitope and interaction orientation. Modeling of the crystal structure interactions onto the cryo-EM structure of GIPR/GIP (POE ID: 7ra3) demonstrates that the anti-GIPR antibodies GIPR-0107, GIPR-2767, 2G10 and GIPG013 make direct contact with the GIP binding site on GIPR and would be direct competitors for the GIPR/GIP interaction (FIG. 8). FIG. 8 shows a model of anti-GIPR antibodies blocking GIPR/GIP interactions. The GIPR/GIP interaction from 7ra3 cryo-EM structure is shown on the left with GIP in dark gray and GIPR in light gray. The ECD and TM-domains are noted and dashed lines are included for referencing. To the right are the comparison of the binding epitopes of the FV domains (space filled) for GIPR-0107, GIPR-2767, GIPG013 and 2G10. The epitope sites of the GIPR-0107 and GIPR-2767 are more distal from the TM-domain whereas the epitope for the GIPG013 and 2G10 are more proximal.

There is a difference in the location of the epitopes though, as the epitopes of GIPR-0107 and GIPR-2767 are more distal from the TM-domain and the antibodies GIPG013 and 2G10 are more proximal. The GIPR-2767 epitope is located within GIPR residues L35-E119, avoiding the GIPR regions nearer the TM domain. The proximity to the TM-domain and the additional extra-cellular loops GIPR may create additional contacts that would be different between human and other species GIPR. For these epitopes it may be difficult for an antibody to have both high antagonist activity to human GIPR/GIP while also being cross-reactive to additional species such as mouse GIPR. This is the case as 2G10 is not cross-reactive and GIPG013 has lower antagonist activity. In contrast, GIPR-2767 has high antagonist activity to human GIPR/GIP and shows significant agonist activity to mouse GIPR/GIP and rat GIPR/GIP. This increase cross-species activity permits a broader range of species utilization in pre-clinical development and toxicity studies.

The GIPR-2767 epitope is unique in that it appears to be the only epitope that allows for direct competition with the GIP peptide binding site, while maintaining spatial distance from the TM-domain and additional extra-cellular loops of GIPR.

Paratope analysis: The paratope of the anti-GIPR antibodies GIPR-0107 and GIPR-2767 along with benchmark antibodies 2G10 (2G10:GIPR-ECD; PDBid: 6dkj, Killion, E. A., et al., Science Translational Medicine, 2018, 10, eaat3392) and GIPG013 (GIPG013:GIPR-ECD; PDBid 4hj0; Ravn, P. et al., The Journal of Biological Chemistry, 2013, 288(27): 19760-19772) to the GIPR-ECD were determined using a structure-based descriptor (SBD) method as described above. Here paratope residues were determined the same way that epitope residues were determined previously. The paratope residues of the interactions of GIPR-0107, GIPR-2767, 2G10, and GIPG013 with GIPR-ECD re shown in tables 22, 23, 24, and 25, respectively. The paratope residues using Kabat notation of GIPR-0107 for GIPR-ECD are H30, H31, H50, 51, H52, H53, H54, H56, H57, H58, H59, H73, H74, H96, H97, H98, H99, H100B, L32, L50 and L95b. The paratope residues using Kabat notation of GIPR-2767 for GIPR-ECD are H30, H31, H33, H52a, H52b, H52c, H53, H95, H97, H99, H100, H100a, H100b, H100c, H100d, H100e, and L32. The paratope residues using Kabat notation of 2G10 for GIPR-ECD are H30, H31, H32, H52, H52a, H53, H54, H55, H56, H57, H59, H64, H65, H68, H71, H96, H97, H98, H99, H100a, L32, L91, L92, L93, and L94. The paratope residues using Kabat notation of GIPG013 for GIPR-ECD are H53, H54, H94, H95, H96, H97, H100a, H101, L31, L32, L34, L46, L49, L50, L53, L55, L56, L91.

The paratope for GIPR-0107 and GIPR-2767 are dominated by almost exclusively by HC interactions with only 3 and 1 LC residues respectively being part of the paratope, in contrast to 2G10, which has 5 including 4 in the LC CDR3, and GIPG013, which has 9 LC paratope residues. GIPR-0107 and GIPR-2767 have germline light-chains which are IGLV1-47*01 and IGKV1-39*01 respectively. Owing to the minimal VL involvement with the epitope, both GIPR-0107 and particularly GIPR-2767 are anticipated to maintain their functionality with a wide variety of VLs, (or absence of VL) regardless of the germline identity or specific residues in the VL CDRs. This allows for simplified pairing in a common-light chain bi-specific IgG that could allow these GIPR binding domains to be paired with other targeted binding domains, or other modalities of antibodies not requiring VH domains.

GIPR-2767-VH-CDR3 binds almost entirely to the GIPR molecule at residues shared with the other tested antibodies (GIPG013, 2G10, GIPR-1017), comprising residues L35, W39, D66, M67, Y68, R113, H115, and E119. All these residues are also bound by GIP and may be important residues for any antagonistic anti-GIPR antibody. GIPR-2767-VH-CDR3 may also bind V69, but this binding may be less critical—the interaction is limited to a BSA of <20A² with GIPR-2767-VH-CDR3-Y100e and does not involve an electrostatic interaction (Y100e's interaction with adjacent residues D66 and Y68 may be more important).

However, GIPR-VH-CDR-1 and GIPR-2767-VH-CDR-2 do not appear to bind these residues to high degree. Instead, it appears that GIPR-VH-CDR-1 and GIPR-2767-VH-CDR-2 exclusively function to anchor GIPR-2767 to GIPR in a slightly different orientation-binding to a near linear region comprising most of residues between Q102-W112 (Q102, G104, S105, D106, Q108, W109, F110, L111, and W112.). GIPR-2767 is in close contact (<3.8A) and forms electrostatic interactions with residues Q102, S105, D106, Q108, W109, L111, and W112 (G104 and G110 are within 3.8A, but are not involved in electrostatic interaction or significantly buried by immediate contact, so may not be as important for this anchor function). Further, GIPR-2767 does not bind to the region Y87, L8, P89, W90 at all, whereas each of GIPG013, 2G10, and GIPR-1017 bind this region. Unlike the epitopes of GIPG013, 2G10, and GIPR-0107, the GIPR-2767 epitope is also limited between residues L35-E119: avoiding the GIPR ECD regions proximal to the cell membrane.

The difference in binding orientation may be the cause of GIPR-2767's increase in signal in in vivo models for target engagement, such as OGTT test, compared to the other tested antibodies that bind closer to the membrane.

TABLE 22
Paratopes sites for GIPR-0107/GIPR-ECD complex predicted by SBD

| | | | | Close contact | |
|---|---|---|---|---|---|
| | | | | to Antigen | |
| | Kabat | BSA >20 | Electrostatic | (<=3.8 | Antigen |
| Type | Site | Angstroms\hat{ }2 | Interactions | Angstroms) | Contacts |
| SER | H30 | | | X | E119 |
| SER | H31 | 40.2 | | X | H115, E119 |
| GLY | H50 | | | X | Y68 |
| ILE | H51 | | | X | Y68 |
| VAL | H52 | 17.0 | H-Bond | X | Y68 |
| ILE | H53 | 91.0 | | X | L88, W90, H115, |
| | | | | | C118, E119, |
| | | | | | N120 |
| PHE | H54 | 115.9 | | X | L35, Y68, V69, |
| | | | | | P85, Y87, L88, |
| | | | | | H115 |
| ILE | H56 | 120.8 | H-Bond | X | L35, Y36, W39, |
| | | | | | M67, Y68, Y87 |
| ALA | H57 | 16.2 | H-Bond | X | Y36, Y68 |
| ASN | H58 | 63.4 | H-Bond | X | W39, M67, Y68 |
| TYR | H59 | | | X | Y36 |
| GLU | H73 | 41.4 | | X | W90, K123 |
| SER | H74 | 20.8 | | | K123 |
| SER | H96 | 30.0 | | X | L111, R113 |
| ASP | H97 | 40.0 | H-Bond, Salt- | X | R101, G110, |
| | | | Bridge | | L111, R113 |
| TYR | H98 | 114.2 | H-Bond | X | D66, Y68, V69, |
| | | | | | V99, R113, H115 |
| VAL | H99 | 21.1 | | X | D66, M67, Y68 |
| HIS | H100B | | | X | L111 |
| TYR | L32 | 49.1 | H-Bond | X | D106, Q108, |
| | | | | | W109 |
| ARG | L50 | 51.8 | H-Bond | X | Q108, W109, |
| | | | | | G110, L111 |
| SER | L95b | 20.8 | | | R43, M67 |

TABLE 23
Paratope sites for GIPR-2676/GIPR-ECD complex predicted by SBD

| | | | | Close contact | |
|---|---|---|---|---|---|
| | | | | to Antigen | |
| | Kabat | BSA >20 | Electrostatic | (<=3.8 | Antigen |
| Type | Site | Angstrom\hat{ }2 | Interactions | Angstroms) | Contacts |
| SER | H30 | 21.4 | Water-mediated H- | | D106, Q108, |
| | | | bond | | W109 |
| SER | H31 | 74.0 | Water-mediated H- | X | D106, Q108, |
| | | | bond | | W109, G110, |
| | | | | | L111 |
| TRP | H33 | 23.8 | | X | W109, L111, |
| | | | | | W112 |
| SER | H52a | 3.8 | Water-mediated H- | | W109, G110, |
| | | | bond | | L111 |
| LYS | H52b | 58.7 | H-bond, Salt-Bridge | X | G104, S105, |
| | | | | | D106 |
| ALA | H52c | 70.1 | | X | Q102, C103, |
| | | | | | G104, Q108, |
| | | | | | W109, G110 |
| ASP | H53 | 60.5 | H-bond, Water- | X | Q102, W109, |
| | | | mediated H-Bond | | G110, L111, |
| | | | | | W112 |
| GLU | H95 | | | X | L111 |
| ILE | H97 | 72.7 | H-bond, Water- | X | D66, L111, |
| | | | mediated H-Bond | | W112, R113 |
| GLY | H99 | 43.7 | Water-mediated H- | X | D66, M67, Y68 |
| | | | bond | | |
| VAL | H100 | | | X | D66, M67, Y68, |
| | | | | | Y87 |
| PRO | H100a | 59.9 | | X | W39, M67, Y68, |
| | | | | | Y87 |
| PHE | H100b | 165.7 | H-bond | X | 135, Y36, W39, |
| | | | | | Y68, Y87, L88, |
| | | | | | P89, W90 |
| LYS | H100c | 26.4 | H-bond, Water- | X | Y68, H115, R119 |
| | | | mediated H-Bond | | |
| GLY | H100d | 22.2 | | X | Y68, R113, H115 |
| TYR | H100e | 142.3 | H-bond | X | D66, Y68, V69, |
| | | | | | W71, V99, R113, |
| | | | | | H115 |
| TYR | L32 | 22.3 | | | H115, E119 |

TABLE 24
Paratope sites for 2G10/GIPR-ECD complex predicted by SBD

| | | | | Close contact | |
|---|---|---|---|---|---|
| | | | | to Antigen | |
| | | BSA >20 | Electrostatic | (<=3.8 | Antigen |
| Type | Site | Angstroms\hat{ }2 | Interactions | Angstroms) | Contacts |
| SER | H30 | 11.7 | H-Bond | X | R101, R113 |
| ASN | H31 | 71.9 | H-Bond, Water- | X | D66, M67, |
| | | | mediated H-Bond | | R101, R113 |
| TYR | H32 | 22.2 | | | M67 |
| TRP | H52 | 54.7 | | | L35, L88, L89, |
| | | | | | W90 |
| PHE | H52a | 57.1 | | X | Y68, R113, |
| | | | | | H115 |
| ASP | H53 | 42.1 | H-Bond, Salt-Bridge | X | H115, E119, |
| | | | | | N120, K123 |
| ALA | H54 | 34.1 | | | K123, E125 |
| SER | H55 | 53.1 | H-Bond, Water- | X | W90, E119, |
| | | | mediated H-Bond | | N120, K123 |
| ASP | H56 | | | X | L128 |
| LYS | H57 | 67.4 | H-Bond, Salt-Bridge, | X | E125, L128, |
| | | | Water-Mediated H- | | D129 |
| | | | Bond | | |
| TYR | H59 | 28.9 | Water-Mediated H- | X | L128, D129, |
| | | | Bond | | L132 |
| LYS | H64 | 31.8 | | | L132 |
| GLY | H65 | 56.2 | | X | L132, 1133 |
| THR | H68 | 43.6 | Water-Mediated H- | | E125, D129, |
| | | | Bond | | L132, I133 |
| ARG | H71 | | Water-Mediated H- | | E125 |
| | | | Bond | | |
| GLN | H96 | 37.6 | H-Bond | X | Y36, W39, M67 |
| ALA | H97 | 36.4 | | X | Y36, W39, M67 |
| ILE | H98 | 57.9 | | X | W39, M67, Y89, |
| | | | | | L89 |
| PHE | H99 | 76.9 | | X | L35, Y36, W39, |
| | | | | | Y87 |
| VAL | H100a | 29.1 | H-Bond | X | A32, Y36 |
| ASN | L32 | 43.4 | H-Bond | X | G33, E34, Y36, |
| | | | | | Q37 |
| TYR | L91 | 38.0 | H-Bond | X | A32, G33, Y36 |
| ASN | L92 | 35.8 | H-Bond | X | T31, A32, G33, |
| | | | | | E34 |
| ASN | L93 | 32.4 | H-Bond | X | Q30, T31, A32, |
| | | | | | G33 |
| TRP | L94 | 40.9 | | X | Q30, T31, A32, |
| | | | | | L35 |

TABLE 25
Paratope sites for GIPG013/GIPR-ECD complex predicted by SBD

| | | | | Close contact | |
|---|---|---|---|---|---|
| | | | | to Antigen | |
| | Kabat | BSA >20 | Electrostatic | (<=3.8 | Antigen |
| Type | Site | Angstroms\hat{ }2 | Interactions | Angstroms) | Contacts |
| GLY | H27 | 45.2 | H-Bond | X | D114, H115 |
| THR | H28 | 20.7 | | X | R113, D114, |
| | | | | | H115 |
| SER | H30 | 23.6 | | | H115, E119, |
| | | | | | N120 |
| SER | H31 | 76.6 | H-Bond | X | L88, W90, H115, |
| | | | | | C118, E119, |
| TYR | H32 | 62.2 | | X | Y68, H115 |
| THR | H53 | 43.6 | H-Bond | X | W90, N120, |
| | | | | | P121 |
| PHE | H54 | 24.4 | | | P89, N120 |
| GLN | H94 | | | X | Y68 |
| GLY | H95 | 5.6 | H-Bond | X | Y68 |
| PRO | H96 | 75.0 | H-Bond | X | W39, M67, Y68, |
| | | | | | Y87 |
| ILE | H97 | 86.4 | | X | L35, Y36, W39, |
| | | | | | Y87, L88 |
| PRO | H100a | 25.8 | | X | Y36, W39, M67 |
| ASP | H101 | 34.1 | | X | M67, Y68 |
| ASN | L31 | 35.3 | H-Bond | X | Y36, E40 |
| THR | L32 | 18.8 | H-Bond | X | Y36, E40 |
| HIS | L34 | 12.6 | H-Bond | X | Y36, E40 |
| LEU | L46 | | | X | M67 |
| TYR | L49 | 72.6 | H-Bond | X | W39, E40, R43, |
| | | | | | Q47, F65, M67 |
| SER | L50 | 25.5 | H-Bond | X | W39, E40 |
| GLN | L53 | 24.0 | | X | Q47 |
| PRO | L55 | 26.0 | | | F65, M67 |
| SER | L56 | 21.2 | | | W109 |
| TRP | L91 | 61.4 | | X | A32, G33, Y36 |

### Example 12. GLP1R Agonist Activity of Peptides

cAMP assay: The in vitro agonist potency of the peptides disclosed herein was evaluated using a cell-based assay measuring cAMP production. CHO cells stably expressing human GLP1R, or both human GIPR and GLP1R, were incubated with increasing concentrations of agonists for 30 minutes at 37° C. Cells were lysed and cAMP was detected using a homogenous time resolved fluorescence detection kit, as described by the manufacturer (Revvity). The percent effect at each compound concentration was calculated relative to the maximal effect achieved with human GLP-1. The EC₅₀ and maximum effect (Eₘₐₓ) values were then determined from the normalized effects and agonist concentrations using a four-parameter logistic equation (GraphPad Prism).

Peptide linker variants were prepared using the GLP-1R agonist peptide disclosed herein and a GGGGS(x3)-K(BrAc) linker. Table 26 shows the SEQ ID NOs for the peptide linker variants.

TABLE 26
Peptide linker variants

| SEQ ID | GLP1 Variant | |
|---|---|---|
| NO. | Description | Total Construct |
| 149 | GLP1(7-37) | H-Aib-EGTFTSDVSSYLEEQAAKE |
| | [Alb8; Glu22] | FIAWLVKGRGGGGSGGGGSGGGGSK |
| | | -AcBr |
| 150 | GLP1(7-37) | H-Aib-EGTFTSDVSSYLE-Aib- |
| | [Alb8; AiB22] | QAAKEFIAWLVKGRGGGGGSGGGG |
| | | SGGGGSK-AcBr |
| 151 | GLP1(7-37) | H-Aib- |
| | [Alb8; | EGTFTSDYSSYLEEQAAKEFIAWL |
| | Tyr16; Glu22] | VKGRGGGGGSGGGGSGGGGSK-AcBr |
| 152 | GLP1(7-37) | H-Aib-EGTFTSDYSSYLE-Aib- |
| | [Alb8; Tyr16; | QAAKEFIAWLVKGRGGGGGSGGGG |
| | Aib22] | SGGGGSK-AcBr |
| 153 | GLP1(7-37) | H-Aib- |
| Bench- | [Alb8; | EGTFTSDVSSYLEEQAAKEFIAWL |
| mark | Glu22; Gly36] | VKGGGGGGGSGGGGSGGGGS |
| | | K-AcBr |
| 154 | GLP1(7-37) | H-Aib- |
| Bench- | [Alb8; Tyr16; | EGTFTSDYSSYLEEQAAKEFIAW |
| mark | Glu22; Gly36] | LVKGGGGGGGSGGGGSGGGGS |
| | | K-AcBr |

Table 27 shows the GLP1R agonist mode EC50 values of the peptides disclosed herein.

TABLE 27
EC₅₀ and Eₘₐₓ values of GLP-1R peptides in GLP1R agonist mode

| | |
|---|---|
| cAMP Assay | GLP1R agonist mode |

| SEQ ID NO. | EC50 (pM) | Eₘₐₓ (%) |
|---|---|---|
| 149 | 2.0 | 106 |
| 150 | 1.9 | 104 |
| 151 | 3.5 | 109 |
| 152 | 2.1 | 107 |
| 153 | 15 | 109 |
| 154 | 8.3 | 122 |
| human GLP-1 40 | 34 | 100 |

### Embodiments

The following non-limiting embodiments provide illustrative examples of the invention, but do not limit the scope of the invention.

E1. An isolated antibody that binds to human gastric inhibitory polypeptide receptor (GIPR).

E2. The antibody of E1, comprising a heavy chain variable region (VH), comprising a CDR-H1 sequence comprising the amino acid sequence of a sequence selected from the group consisting of SEQ ID NO: 15, SEQ ID NO: 16, and SEQ ID NO: 17; a CDR-H2 sequence comprising the amino acid sequence of a sequence selected from the group consisting of SEQ ID NO: 18 and SEQ ID NO: 19; and a CDR-H3 sequence comprising the amino acid sequence of SEQ ID NO: 20.

E3. The antibody of any one of E1-E2, comprising a VH, comprising a CDR-H1 sequence comprising the amino acid sequence of a sequence selected from the group consisting of SEQ ID NO: 15, SEQ ID NO: 16, SEQ ID NO: 17, SEQ ID NO: 2, SEQ ID NO: 3, and SEQ ID NO: 4; a CDR-H2 sequence comprising the amino acid sequence SEQ ID NO: 18, SEQ ID NO: 19, SEQ ID NO: 5, and SEQ ID NO: 6; and a CDR-H3 sequence comprising the amino acid sequence of a sequence selected from the group consisting of a sequence selected from the group consisting of SEQ ID NO: 20 and SEQ ID NO: 7; and comprising a light chain variable region (VL) comprising a CDR-L1 sequence comprising the amino acid sequence of a sequence selected from the group consisting of SEQ ID NO: 23, and SEQ ID NO: 10; a CDR-L2 sequence comprising the amino acid sequence of a sequence selected from the group consisting of SEQ ID NO: 24, and SEQ ID NO: 11; and a CDR-L3 sequence comprising the amino acid sequence of a sequence selected from the group consisting of SEQ ID NO: 25, and SEQ ID NO: 12.

E4. The antibody of any one of E1-E3, comprising a VH, comprising a CDR-H1 sequence comprising the amino acid sequence of a sequence selected from the group consisting of SEQ ID NO: 15, SEQ ID NO: 16, SEQ ID NO: 17; a CDR-H2 sequence comprising the amino acid sequence SEQ ID NO: 18, and SEQ ID NO: 19; and a CDR-H3 sequence comprising the amino acid sequence of SEQ ID NO: 20; and comprising a VL comprising a CDR-L1 sequence comprising the amino acid sequence of SEQ ID NO: 23; a CDR-L2 sequence comprising the amino acid sequence of SEQ ID NO: 24; and a CDR-L3 sequence comprising the amino acid sequence of a sequence of SEQ ID NO: 25.

E5. The antibody of any one of E1-E4, comprising a heavy chain variable region (VH), comprising a CDR-H1 sequence comprising the amino acid sequence of a sequence selected from the group consisting of SEQ ID NO: 2, SEQ ID NO: 3, SEQ ID NO: 4; a CDR-H2 sequence comprising the amino acid sequence SEQ ID NO: 5, and SEQ ID NO: 6; and a CDR-H3 sequence comprising the amino acid sequence of SEQ ID NO: 7; and comprising a light chain variable region (VL) comprising a CDR-L1 sequence comprising the amino acid sequence of SEQ ID NO: 10; a CDR-L2 sequence comprising the amino acid sequence of SEQ ID NO: 11; and a CDR-L3 sequence comprising the amino acid sequence of SEQ ID NO: 12.

E6. The antibody of any one of E1-E5, comprising a heavy chain and a light chain, and the heavy chain comprises a heavy chain variable region (VH) and the light chain comprises a light chain variable region (VL), wherein
(i) the VH comprises: a complementary determining region (CDR)-H1 comprising an amino acid sequence of SEQ ID NO: 2, SEQ ID NO: 3, SEQ ID NO: 4, SEQ ID NO: 15, SEQ ID NO: 16, or SEQ ID NO: 17; a VH CDR-H2 comprising an amino acid sequence of SEQ ID NO: 5, SEQ ID NO: 6, SEQ ID NO: 18, or SEQ ID NO: 19; and a VH CDR-H3 comprising an amino acid sequence of SEQ ID NO: 7 or SEQ ID NO: 20; and
(ii) the VL comprises a CDR-L1 comprising an amino acid sequence of SEQ ID NO: 10 or SEQ ID NO: 23; a CDR-L2 comprising an amino acid sequence of SEQ ID NO: 11 or SEQ ID NO: 24; and a CDR-L3 comprising an amino acid sequence of SEQ ID NO: 12 or SEQ ID NO: 25.

E7. The antibody of any one of E1-E6, comprising CDRs selected from the one of the groups in the following list:
(i) a CDR-H1 sequence comprising the amino acid sequence of SEQ ID NO: 15; a CDR-H2 sequence comprising the amino acid sequence of SEQ ID NO: 18; a CDR-H3 sequence comprising the amino acid sequence of SEQ ID NO: 20, wherein the numbering is according to Chothia;
(ii) a CDR-H1 sequence comprising the amino acid sequence of SEQ ID NO: 16; a CDR-H2 sequence comprising the amino acid sequence of SEQ ID NO: 19; a CDR-H3 sequence comprising the amino acid sequence of SEQ ID NO: 20, wherein the numbering is according to Kabat;
(iii) a CDR-H1 sequence comprising the amino acid sequence of SEQ ID NO: 15; a CDR-H2 sequence comprising the amino acid sequence of SEQ ID NO: 18; a CDR-H3 sequence comprising the amino acid sequence of SEQ ID NO: 20; a CDR-L1 sequence comprising the amino acid sequence of SEQ ID NO: 23; a CDR-L2 sequence comprising the amino acid sequence of SEQ ID NO: 24; a CDR-L3 sequence comprising the amino acid sequence of SEQ ID NO: 25, wherein the numbering is according to Chothia; and
(iv) a CDR-H1 sequence comprising the amino acid sequence of SEQ ID NO: 16; a CDR-H2 sequence comprising the amino acid sequence of SEQ ID NO: 19; a CDR-H3 sequence comprising the amino acid sequence of SEQ ID NO: 20; a CDR-L1 sequence comprising the amino acid sequence of SEQ ID NO: 23; a CDR-L2 sequence comprising the amino acid sequence of SEQ ID NO: 24; a CDR-L3 sequence comprising the amino acid sequence of SEQ ID NO: 25, wherein the numbering is according to Kabat;
(v) a CDR-H1 sequence comprising the amino acid sequence of SEQ ID NO: 2; a CDR-H2 sequence comprising the amino acid sequence of SEQ ID NO: 5; a CDR-H3 sequence comprising the amino acid sequence of SEQ ID NO: 7, wherein the numbering is according to Chothia;
(vi) a CDR-H1 sequence comprising the amino acid sequence of SEQ ID NO: 3; a CDR-H2 sequence comprising the amino acid sequence of SEQ ID NO: 6; a CDR-H3 sequence comprising the amino acid sequence of SEQ ID NO: 7, wherein the numbering is according to Kabat;
(vii) a CDR-H1 sequence comprising the amino acid sequence of SEQ ID NO: 2; a CDR-H2 sequence comprising the amino acid sequence of SEQ ID NO: 5; a CDR-H3 sequence comprising the amino acid sequence of SEQ ID NO: 7; a CDR-L1 sequence comprising the amino acid sequence of SEQ ID NO: 10; a CDR-L2 sequence comprising the amino acid sequence of SEQ ID NO: 11; a CDR-L3 sequence comprising the amino acid sequence of SEQ ID NO: 12, wherein the numbering is according to Chothia; and
(viii) a CDR-H1 sequence comprising the amino acid sequence of SEQ ID NO: 3; a CDR-H2 sequence comprising the amino acid sequence of SEQ ID NO: 6; a CDR-H3 sequence comprising the amino acid sequence of SEQ ID NO: 7; a CDR-L1 sequence comprising the amino acid sequence of SEQ ID NO: 10; a CDR-L2 sequence comprising the amino acid sequence of SEQ ID NO: 11; a CDR-L3 sequence comprising the amino acid sequence of SEQ ID NO: 12, wherein the numbering is according to Kabat; and

E8. The antibody of any one of E1-E7, comprising a CDR-H1 sequence comprising the amino acid sequence of SEQ ID NO: 15; a CDR-H2 sequence comprising the amino acid sequence of SEQ ID NO: 18; a CDR-H3 sequence comprising the amino acid sequence of SEQ ID NO: 20; a CDR-L1 sequence comprising the amino acid sequence of SEQ ID NO: 23; a CDR-L2 sequence comprising the amino acid sequence of SEQ ID NO: 24; a CDR-L3 sequence comprising the amino acid sequence of SEQ ID NO: 25, wherein the numbering is according to Chothia.

E9. The antibody of any one of E1-E8, comprising a CDR-H1 sequence comprising the amino acid sequence of SEQ ID NO: 16; a CDR-H2 sequence comprising the amino acid sequence of SEQ ID NO: 19; a CDR-H3 sequence comprising the amino acid sequence of SEQ ID NO: 20; a CDR-L1 sequence comprising the amino acid sequence of SEQ ID NO: 23; a CDR-L2 sequence comprising the amino acid sequence of SEQ ID NO: 24; a CDR-L3 sequence comprising the amino acid sequence of SEQ ID NO: 25, wherein the numbering is according to Kabat.

E10. The antibody of any one of E1-E9, comprising a heavy chain and a light chain, and the heavy chain comprises a heavy chain variable region (VH) and the light chain comprises a light chain variable region (VL), wherein
(i) the VH comprises a complementary determining region (CDR)-H1 having an amino acid sequence of SEQ ID NO: 15, SEQ ID NO: 16, or SEQ ID NO: 17, a CDR-H2 having an amino acid sequence of SEQ ID NO: 18 or SEQ ID NO: 19, and a CDR-H3 having an amino acid sequence of SEQ ID NO: 20; and
(ii) the VL comprises a CDR-L1 having an amino acid sequence of SEQ ID NO: 23, a CDR-L2 having an amino acid sequence of SEQ ID NO: 24, and a CDR-L3 having an amino acid sequence of SEQ ID NO: 25.

E11. The antibody of any one of E1-E10, comprising a heavy chain variable region (VH) and a light chain variable region (VL), wherein the VH comprises a CDR-H1 comprising an amino acid sequence comprising 0, 1 or 2 substitutions, deletions, or additions to the amino acid sequence of SEQ ID NO: 15; a CDR-H2 comprising an amino acid sequence comprising 0, 1 or 2 substitutions, deletions, or additions to the amino acid sequence of SEQ ID NO: 18; and a CDR-H3 comprising an amino acid sequence comprising 0, 1 or 2 substitutions, deletions, or additions to the amino acid sequence of SEQ ID NO: 20; and wherein the VL comprises a CDR-L1 comprising an amino acid sequence comprising 0, 1 or 2 substitutions, deletions, or additions to the amino acid sequence of SEQ ID NO: 23; a CDR-L2 comprising an amino acid sequence comprising 0, 1 or 2 substitutions, deletions, or additions to the amino acid sequence of SEQ ID NO: 24; and a CDR-L3 comprising an amino acid sequence comprising 0, 1 or 2 substitutions, deletions, or additions to the amino acid sequence of SEQ ID NO: 25; wherein the numbering is according to Chothia.

E12. The antibody of any one of E1-E11, comprising a heavy chain variable region (VH) and a light chain variable region (VL), wherein the VH comprises a CDR-H1 comprising an amino acid sequence comprising 0, 1 or 2 substitutions, deletions, or additions to the amino acid sequence of SEQ ID NO: 16; a CDR-H2 comprising an amino acid sequence comprising 0, 1 or 2 substitutions, deletions, or additions to the amino acid sequence of SEQ ID NO: 19; and a CDR-H3 comprising an amino acid sequence comprising 0, 1 or 2 substitutions, deletions, or additions to the amino acid sequence of SEQ ID NO: 20; and wherein the VL comprises a CDR-L1 comprising an amino acid sequence comprising 0, 1 or 2 substitutions, deletions, or additions to the amino acid sequence of SEQ ID NO: 23; a CDR-L2 comprising an amino acid sequence comprising 0, 1 or 2 substitutions, deletions, or additions to the amino acid sequence of SEQ ID NO: 24; and a CDR-L3 comprising an amino acid sequence comprising 0, 1 or 2 substitutions, deletions, or additions to the amino acid sequence of SEQ ID NO: 25; wherein the numbering is according to Kabat.

E13. The antibody of any one of E1-E12, comprising a heavy chain variable region (VH) and a light chain variable region (VL), wherein the VH comprises a CDR-H1 comprising an amino acid sequence of SEQ ID NO: 15; a CDR-H2 comprising an amino acid sequence comprising of SEQ ID NO: 18; and a CDR-H3 comprising an amino acid sequence of SEQ ID NO: 20; and wherein the VL comprises a CDR-L1 comprising an amino acid sequence comprising 0, 1 or 2 substitutions, deletions, or additions to the amino acid sequence of SEQ ID NO: 23; a CDR-L2 comprising an amino acid sequence comprising 0, 1 or 2 substitutions, deletions, or additions to the amino acid sequence of SEQ ID NO: 24; and a CDR-L3 comprising an amino acid sequence comprising 0, 1 or 2 substitutions, deletions, or additions to the amino acid sequence of SEQ ID NO: 25; wherein the numbering is according to Chothia.

E14. The antibody of any one of E1-E13, comprising a heavy chain variable region (VH) and a light chain variable region (VL), wherein the VH comprises a CDR-H1 comprising an amino acid sequence comprising of SEQ ID NO: 16; a CDR-H2 comprising an amino acid sequence of SEQ ID NO: 19; and a CDR-H3 comprising an amino acid sequence of SEQ ID NO: 20; and wherein the VL comprises a CDR-L1 comprising an amino acid sequence comprising 0, 1 or 2 substitutions, deletions, or additions to the amino acid sequence of SEQ ID NO: 23; a CDR-L2 comprising an amino acid sequence comprising 0, 1 or 2 substitutions, deletions, or additions to the amino acid sequence of SEQ ID NO: 24; and a CDR-L3 comprising an amino acid sequence comprising 0, 1 or 2 substitutions, deletions, or additions to the amino acid sequence of SEQ ID NO: 25; wherein the numbering is according to Kabat.

E15. The antibody of any one of E1-E14, comprising CDRs selected from the one of the groups in the following list:
(i) comprising the CDR-H1, CDR-H2, and CDR-H3 sequences of SEQ ID NO: 14, and the CDR-L1, CDR-L2, and CDR-L3 sequences of SEQ ID NO: 22;
(ii) comprising the CDR-H1, CDR-H2, and CDR-H3 sequences of SEQ ID NO: 1, and the CDR-L1, CDR-L2, and CDR-L3 sequences of SEQ ID NO: 9.

E16. The antibody of any one of E1-E15, comprising a VH framework and a VL framework, wherein one or both of the VH framework and a VL framework is derived from a human germline VH sequence.

E17. The antibody of any one of E1-E16, comprising a VH framework sequence and a VL framework sequence wherein one or both of the VH framework sequence and the VL framework sequence is at least 90%, 95%, 98%, 99%, or 100% identical to the human germline sequence from which it was derived.

E18. The antibody of any one of E1-E19, wherein the VH comprises the amino acid sequence of SEQ ID NO: 14 or a variant of SEQ ID NO: 14 thereof comprising one to four amino acid substitutions at residues that are not within a CDR, and the VL comprises the amino acid sequence of SEQ ID NO: 22 or a variant of SEQ ID NO: 22 thereof comprising one to four amino acid substitutions at residues that are not within a CDR.

E19. The antibody of any one of E1-E18, comprising a VH comprising an amino acid sequence at least 90%, 91%, 92%, 93%, 94%, 95%, 96%, 97%, 98%, 99%, or 100% identical to SEQ ID NO: 14, and comprising a VL comprising an amino acid sequence at least 90%, 91%, 92%, 93%, 94%, 95%, 96%, 97%, 98%, 99%, or 100% identical to SEQ ID NO: 22.

E20. The antibody of any one of E1-E19, wherein the amino acid sequence of the VH is at least 90%, 91%, 92%, 93%, 94%, 95%, 96%, 97%, 98%, 99%, or 100% identical to the amino acid sequence of SEQ ID NO: 14, and amino acid sequence of the VL is at least 90%, 91%, 92%, 93%, 94%, 95%, 96%, 97%, 98%, 99%, or 100% identical to the amino acid sequence of SEQ ID NO: 22, and wherein the VH comprises a CDR-H1 sequence comprising the amino acid sequence of SEQ ID NO: 15; a CDR-H2 sequence comprising the amino acid sequence of SEQ ID NO: 18; a CDR-H3 sequence comprising the amino acid sequence of SEQ ID NO: 20; and wherein the VL comprises a CDR-L1 sequence comprising the amino acid sequence of SEQ ID NO: 23; a CDR-L2 sequence comprising the amino acid sequence of SEQ ID NO: 24, and a CDR-L3 sequence comprising the amino acid sequence of SEQ ID NO: 25, according to Chothia numbering.

E21. The antibody of any one of E1-E20, wherein the amino acid sequence of the VH is at least 90%, 91%, 92%, 93%, 94%, 95%, 96%, 97%, 98%, 99%, or 100% identical to the amino acid sequence of SEQ ID NO: 14, and amino acid sequence of the VL is at least 90%, 91%, 92%, 93%, 94%, 95%, 96%, 97%, 98%, 99%, or 100% identical to the amino acid sequence of SEQ ID NO: 22, and wherein the VH comprises a CDR-H1 sequence comprising the amino acid sequence of SEQ ID NO: 16; a CDR-H2 sequence comprising the amino acid sequence of SEQ ID NO: 19; a CDR-H3 sequence comprising the amino acid sequence of SEQ ID NO: 20; and wherein the VL comprises a CDR-L1 sequence comprising the amino acid sequence of SEQ ID NO: 23; a CDR-L2 sequence comprising the amino acid sequence of SEQ ID NO: 24, and a CDR-L3 sequence comprising the amino acid sequence of SEQ ID NO: 25, according to Kabat numbering.

E22. The antibody of any one of E1-E21, comprising a VH comprising an amino acid sequence selected from the group consisting of SEQ ID NO: 14 and SEQ ID NO: 1, and comprising a VL comprising an amino acid sequence selected from the group consisting of SEQ ID NO: 22 and SEQ ID NO: 9.

E23. The antibody of any one of E1-E22, comprising a heavy chain variable region (VH) and a light chain variable region (VL) selected from the one of the groups in the following list:
i) a VH comprising the amino acid sequence of SEQ ID NO: 14 and a VL comprising the amino acid sequence of SEQ ID NO: 22; and
ii) a VH comprising the amino acid sequence of SEQ ID NO: 1 and a VL comprising the amino acid sequence of SEQ ID NO: 9.

E24. The antibody of any one of E1-E23, wherein the VH comprises the amino acid sequence shown in SEQ ID NO: 14, and the VL comprises the amino acid sequence shown in SEQ ID NO: 22.

E25. The antibody of any one of E1-E23, wherein the VH comprises the amino acid sequence shown in SEQ ID NO: 1, and the VL comprises the amino acid sequence shown in SEQ ID NO: 9.

E26. The antibody of any one of E1-E25, comprising one or both of a VH sequence encoded by a polynucleotide comprising the nucleic acid sequence of SEQ ID NO; and a VL sequence encoded by a polynucleotide comprising the nucleic acid sequence of SEQ ID NO

E27. The antibody of any one of E1-E26, wherein the antibody is human, humanized or chimeric.

E28. The antibody of any one of E1-E27, wherein the antibody is one or more selected from the group consisting of a single chain antibody, a Fab fragment, a F(ab)2 fragment, a Fv fragment, a tetrameric antibody, a tetravalent antibody, a bi-specific antibody, a multispecific antibody, a domain-specific antibody, a single domain antibody, and a fusion protein.

E29. The antibody of any one of E1-E28, wherein the antibody is linked to a second functional moiety having a different binding specificity than said antibody.

E30. The antibody of any one of E1-E28, wherein the antibody is not linked to a second functional moiety having a different binding specificity than said antibody.

E31. The antibody of any one of E1-E30, further comprising an Fc domain, wherein the Fc domain is an isotype selected from the group consisting of IgG, IgG1, IgG2, IgG3, IgG4, IgA, IgA1, IgA2, IgD, IgE, and IgM.

E32. The antibody of any one of E1-E31, comprising a Fc domain of an isotype of IgG.

E33. The antibody of any one of E1-E32, comprising a Fc domain of an isotype of IgG1.

E34. The antibody of any one of E1-E33, comprising a HC comprising an amino acid sequence at least 90%, 91%, 92%, 93%, 94%, 95%, 96%, 97%, 98%, 99%, or 100% identical to SEQ ID NO: 21, and a LC comprising an amino acid sequence at least 90%, 91%, 92%, 93%, 94%, 95%, 96%, 97%, 98%, 99%, or 100% identical to SEQ ID NO: 26.

E35. The antibody of any one of E1-E34, comprising one or both of a HC sequence comprising an amino acid sequence selected from the group consisting of SEQ ID NO: 21, SEQ ID NO: 8, SEQ ID NO: 27, SEQ ID NO: 28, SEQ ID NO: 30, SEQ ID NO: 32, SEQ ID NO: 34, SEQ ID NO: 35, SEQ ID NO: 36, SEQ ID NO: 37, SEQ ID NO: 38, and SEQ ID NO: 39; and a LC sequence comprising an amino acid sequence selected from the group consisting of SEQ ID NO: 26, SEQ ID NO: 13, SEQ ID NO: 29, and SEQ ID NO: 31.

E36. The antibody of any one of E1-E34, comprising one or both of a HC sequence comprising an amino acid sequence selected from the group consisting of SEQ ID NO: 21, SEQ ID NO: 8, SEQ ID NO: 30, SEQ ID NO: 32, SEQ ID NO: 34, SEQ ID NO: 35, SEQ ID NO: 36, SEQ ID NO: 37, SEQ ID NO: 38, and SEQ ID NO: 39; and a LC sequence comprising an amino acid sequence selected from the group consisting of SEQ ID NO: 26, SEQ ID NO: 13, SEQ ID NO: 29, and SEQ ID NO: 31.

E37. The antibody of any one of E1-E36, comprising a heavy chain (HC) and a light chain (LC) selected from one of the groups in the following list:
i) a HC comprising the amino acid sequence of SEQ ID NO: 21 and a LC comprising the amino acid sequence of SEQ ID NO: 26;
ii) a HC comprising the amino acid sequence of SEQ ID NO: 8 and a LC comprising the amino acid sequence of SEQ ID NO: 13;
   E37. The antibody of any one of E1-E36, wherein the HC comprises an amino acid sequence of SEQ ID NO: 21, and the LC comprises an amino acid sequence of SEQ ID NO: 26.

E38. The antibody of any one of E1-E37, wherein
(i) the HC comprises an amino acid sequence of SEQ ID NO: 30, SEQ ID NO: 32, SEQ ID NO: 37, SEQ ID NO: 38, or SEQ ID NO: 39, and the LC comprises an amino acid sequence of SEQ ID NO: 26; or
(ii) the HC comprise an amino acid sequence of SEQ ID NO: 21, and the LC comprises an amino acid sequence of SEQ ID NO: 31.

E39. The antibody of any one of E1-E38, wherein the HC comprises an amino acid sequence of SEQ ID NO: 8, and the LC comprises an amino acid sequence of SEQ ID NO: 13.

E40. The antibody of any one of E1-E39, wherein the HC comprises an amino acid sequence of SEQ ID NO: 27 or SEQ ID NO: 28, and the LC comprises an amino acid sequence of SEQ ID NO:13.

E41. The antibody of any one of E1-E40, wherein the HC comprises an amino acid sequence of SEQ ID NO: 8, and the LC comprises an amino acid sequence of SEQ ID NO: 29.

E42. The antibody of any one of E1-E41, wherein
(i) the HC comprises an amino acid sequence of SEQ ID NO: 27, SEQ ID NO: 28, SEQ ID NO: 34, SEQ ID NO: 35, or SEQ ID NO: 36, and the LC comprises an amino acid sequence of SEQ ID NO: 13; or
(ii) the HC comprises an amino acid sequence of SEQ ID NO: 8, and the LC comprises an amino acid sequence of SEQ ID NO: 29.

E43. The antibody of any one of E1-E42, wherein the antibody is a full-length human or humanized IgG antibody comprising two heavy chains and two light chains, wherein the two heavy chains each have a first pair of identical amino acid sequences, and the two light chains each have a second pair of identical amino acid sequences.

E44. The antibody of any one of E1-E43, comprising a LC comprising an amino acid sequence of SEQ ID NO: 8; and a HC comprising an amino acid sequence of SEQ ID NO: 13.

E45. The antibody of any one of E1-E43, comprising a light chain comprising an amino acid sequence of SEQ ID NO: 21; and a heavy chain comprising an amino acid sequence of SEQ ID NO: 26.

E46. The antibody of any one of E1-E45, wherein the antibody binds to an epitope on human GIPR comprising one or more residues selected from the group consisting of L35, W39, D66, M67, Y68, R113, H115, and E119, wherein the numbering is of SEQ ID NO: 148.

E47. An isolated antibody that binds to human gastric inhibitory polypeptide receptor (GIPR)., wherein the antibody binds to an epitope on human GIPR comprising one or more residues selected from the group consisting of L35, W39, D66, M67, Y68, R113, H115, and E119, wherein the numbering is of SEQ ID NO: 148.

E48. The antibody of any one of E1-E47, wherein the antibody binds to an epitope on human GIPR comprising two or more residues selected from the group consisting of L35, W39, D66, M67, Y68, R113, H115, and E119, wherein the numbering is of SEQ ID NO: 148.

E49. The antibody of any one of E1-E48, wherein the antibody binds to an epitope on human GIPR comprising three or more residues selected from the group consisting of L35, W39, D66, M67, Y68, R113, H115, and E119, wherein the numbering is of SEQ ID NO: 148.

E50. The antibody of any one of E1-E49, wherein the antibody binds to an epitope on human GIPR comprising four or more residues selected from the group consisting of L35, W39, D66, M67, Y68, R113, H115, and E119, wherein the numbering is of SEQ ID NO: 148.

E51. The antibody of any one of E1-E50, wherein the antibody binds to an epitope on human GIPR comprising five or more residues selected from the group consisting of L35, W39, D66, M67, Y68, R113, H115, and E119, wherein the numbering is of SEQ ID NO: 148.

E52. The antibody of any one of E1-E51, wherein the antibody binds to an epitope on human GIPR comprising six or more residues selected from the group consisting of L35, W39, D66, M67, Y68, R113, H115, and E119, wherein the numbering is of SEQ ID NO: 148.

E53. The antibody of any one of E1-E52, wherein the antibody binds to an epitope on human GIPR comprising seven residues selected from the group consisting of L35, W39, D66, M67, Y68, R113, H115, and E119, wherein the numbering is of SEQ ID NO: 148.

E54. The antibody of any one of E1-E53, wherein the antibody binds to an epitope on human GIPR comprising L35, W39, D66, M67, Y68, R113, H115, and E119, wherein the numbering is of SEQ ID NO: 148.

E55. The antibody of any one of E1-E54, wherein the antibody binds to an epitope on human GIPR comprising V69, wherein the numbering is of SEQ ID NO: 148.

E56. The antibody of any one of E1-E55, wherein the antibody binds to an epitope on human GIPR comprising L35, W39, D66, M67, Y68, V69, R113, H115, and E119, wherein the numbering is of SEQ ID NO: 148.

E57. The antibody of any one of E1-E56, wherein the antibody binds to an epitope on human GIPR comprising one or more residues selected from the group consisting of Q102, S105, D106, Q108, W109, L111, and W112, wherein the numbering is of SEQ ID NO: 148.

E58. The antibody of any one of E1-E57, wherein the antibody binds to an epitope on human GIPR comprising two or more residues selected from the group consisting of Q102, S105, D106, Q108, W109, L111, and W112, wherein the numbering is of SEQ ID NO: 148.

E59. The antibody of any one of E1-E58, wherein the antibody binds to an epitope on human GIPR comprising three or more residues selected from the group consisting of Q102, S105, D106, Q108, W109, L111, and W112, wherein the numbering is of SEQ ID NO: 148.

E60. The antibody of any one of E1-E59, wherein the antibody binds to an epitope on human GIPR comprising four or more residues selected from the group consisting of Q102, S105, D106, Q108, W109, L111, and W112, wherein the numbering is of SEQ ID NO: 148.

E61. The antibody of any one of E1-E60, wherein the antibody binds to an epitope on human GIPR comprising five or more residues selected from the group consisting of Q102, S105, D106, Q108, W109, L111, and W112, wherein the numbering is of SEQ ID NO: 148.

E62. The antibody of any one of E1-E61, wherein the antibody binds to an epitope on human GIPR comprising six residues selected from the group consisting of Q102, S105, D106, Q108, W109, L111, and W112, wherein the numbering is of SEQ ID NO: 148.

E63. The antibody of any one of E1-E62, wherein the antibody binds to an epitope on human GIPR comprising Q102, S105, D106, Q108, W109, L111, and W112, wherein the numbering is of SEQ ID NO: 148.

E64. The antibody of any one of E1-E63, wherein the antibody binds to an epitope on human GIPR comprising G104, wherein the numbering is of SEQ ID NO: 148.

E65. The antibody of any one of E1-E64, wherein the antibody binds to an epitope on human GIPR comprising G110, wherein the numbering is of SEQ ID NO: 148.

E66. The antibody of any one of E1-E65, wherein the antibody binds to an epitope on human GIPR comprising Q102, G104, S105, D106, Q108, W109, G110, L111, and W112, wherein the numbering is of SEQ ID NO: 148.

E67. The antibody of any one of E1-E66, wherein the antibody binds to an epitope on human GIPR comprising L35, W39, D66, M67, Y68, Q102, S105, D106, Q108, W109, L111, and W112, R113, H115, and E119, wherein the numbering is of SEQ ID NO: 148.

E68. The antibody of any one of E1-E67, wherein the antibody binds to an epitope on human GIPR comprising L35, W39, D66, M67, Y68, V69, Q102, S105, D106, Q108, W109, 111, and W112, R113, H115, and E119, wherein the numbering is of SEQ ID NO: 148.

E69. The antibody of any one of E1-E68, wherein the antibody binds to an epitope on human GIPR comprising L35, W39, D66, M67, Y68, Q102, G104, S105, D106, Q108, W109, G110, 111, and W112, R113, H115, and E119, wherein the numbering is of SEQ ID NO: 148.

E70. The antibody of any one of E1-E69, wherein the antibody binds to an epitope on human GIPR comprising L35, W39, D66, M67, Y68, V69, Q102, G104, S105, D106, Q108, W109, G110, 111, and W112, R113, H115, and E119, wherein the numbering is of SEQ ID NO: 148.

E71. The antibody of any one of E1-E70, wherein the antibody does not bind one or more residues selected from the group consisting of Y87, L8, P89, and W90, wherein the numbering is of SEQ ID NO: 148.

E72. The antibody of any one of E1-E71, wherein the antibody does not bind two or more residues selected from the group consisting of Y87, L8, P89, and W90, wherein the numbering is of SEQ ID NO: 148.

E73. The antibody of any one of E1-E72, wherein the antibody does not bind three residues selected from the group consisting of Y87, L8, P89, and W90, wherein the numbering is of SEQ ID NO: 148.

E74. The antibody of any one of E1-E73, wherein the antibody does not bind any of Y87, L8, P89, and W90, wherein the numbering is of SEQ ID NO: 148.

E75. The antibody of any one of E1-E74, wherein the antibody binds to an epitope on human GIPR located between residue L35 and E119, wherein the numbering is of SEQ ID NO: 148.

E76. The antibody of any one of E1-E75, wherein the antibody binds to an epitope on human GIPR that does not comprise any residues between 1-34, wherein the numbering is of SEQ ID NO: 148.

E77. The antibody of any one of E1-E76, wherein the antibody binds to an epitope on human GIPR that does not comprise any residues numbering above 120, wherein the numbering is of SEQ ID NO: 148.

E78. The antibody of any one of E1-E77, wherein the antibody binds to an epitope on human GIPR that does not comprise one or more residues selected from the group consisting of Q30, T31, A32, G33, and E34.

E79. The antibody of any one of E1-E78, comprising residue VH-CDR1-S30.

E80. The antibody of any one of E1-E79, comprising residue VH-CDR1-S31.

E81. The antibody of any one of E1-E80, comprising residue VH-CDR1-W33.

E82. The antibody of any one of E1-E81, comprising residue VH-CDR2-S52a.

E83. The antibody of any one of E1-E82, comprising residue VH-CDR2-K52b.

E84. The antibody of any one of E1-E83, comprising residue VH-CDR2-A52c.

E85. The antibody of any one of E1-E84, comprising residue VH-CDR2-D53.

E86. The antibody of any one of E1-E85, comprising residue VH-CDR3-Q95

E87. The antibody of any one of E1-E86, comprising residue VH-CDR3-197.

E88. The antibody of any one of E1-E87, comprising residue VH-CDR3-G99.

E89. The antibody of any one of E1-E88, comprising residue VH-CDR3-V100.

E90. The antibody of any one of E1-E89, comprising residue VH-CDR3-P100a.

E91. The antibody of any one of E1-E90, comprising residue VH-CDR3-F100b.

E92. The antibody of any one of E1-E91, comprising residue VH-CDR3-K100c.

E93. The antibody of any one of E1-E92, comprising residue VH-CDR3-G100d.

E94. The antibody of any one of E1-E93, comprising residue VH-CDR3-Y100e.

E95. The antibody of any one of E1-E94, comprising residue VL-CDR1-Y32.

E96. The antibody of any one of E1-E95, comprising one or more residues selected from the group consisting of VH-CDR1-S30, VH-CDR1-S31, VH-CDR1-W33, VH-CDR2-S52a, VH-CDR2-K52b, VH-CDR2-A52c, VH-CDR2-D53, VH-CDR3-Q9, VH-CDR3-197, VH-CDR3-G99, VH-CDR3-V100, VH-CDR3-P100a, VH-CDR3-F100b, VH-CDR3-K100c, VH-CDR3-G100d, and VH-CDR3-Y100e.

E97. The antibody of any one of E1-E96, comprising VH-CDR1-S30, VH-CDR1-S31, VH-CDR1-W33, VH-CDR2-S52a, VH-CDR2-K52b, VH-CDR2-A52c, VH-CDR2-D53, VH-CDR3-Q9, VH-CDR3-197, VH-CDR3-G99, VH-CDR3-V100, VH-CDR3-P100a, VH-CDR3-F100b, VH-CDR3-K100c, VH-CDR3-G100d, and VH-CDR3-Y100e.

E98. The antibody of any one of E1-E97, comprising VH-CDR1-S30, VH-CDR1-S31, VH-CDR1-W33, VH-CDR2-S52a, VH-CDR2-K52b, VH-CDR2-A52c, VH-CDR2-D53, VH-CDR3-Q9, VH-CDR3-197, VH-CDR3-G99, VH-CDR3-V100, VH-CDR3-P100a, VH-CDR3-F100b, VH-CDR3-K100c, VH-CDR3-G100d, VH-CDR3-Y100e, and VL-CDR1-Y32.

E99. The antibody of any one of E1-E98, wherein the VH comprises an amino acid sequence of SEQ ID NO: 14, and the VL comprises an amino acid sequence of SEQ ID NO: 22.

E100. The antibody of any one of E1-E99, wherein the heavy chain comprises an amino acid sequence of SEQ ID NO: 21, and the light chain comprises an amino acid sequence of SEQ ID NO: 26.

E101. The antibody of any one of E1-E100, wherein the (i) the heavy chain comprises an amino acid sequence of SEQ ID NO: 30, SEQ ID NO: 32, SEQ ID NO: 37, SEQ ID NO: 38, or SEQ ID NO: 39, and the light chain comprises an amino acid sequence of SEQ ID NO: 26; or (ii) the heavy chain comprise an amino acid sequence of SEQ ID NO: 21, and the light chain comprises an amino acid sequence of SEQ ID NO: 31.

E102. An isolated antibody that binds to human gastric inhibitory polypeptide receptor (GIPR), comprising a heavy chain and a light chain, and the heavy chain comprises a heavy chain variable region (VH) and the light chain comprises a light chain variable region (VL), wherein (i) the VH comprises: a complementary determining region (CDR)-H1 having an amino acid sequence of SEQ ID NO: 2, SEQ ID NO: 3, or SEQ ID NO: 4, a CDR-H2 having an amino acid sequence of SEQ ID NO: 5 or SEQ ID NO: 6, and a CDR-H3 having an amino acid sequence of SEQ ID NO: 7; and (ii) the VL comprises: a CDR-L1 having an amino acid sequence of SEQ ID NO: 10, a CDR-L2 having an amino acid sequence of SEQ ID NO: 11, and a CDR-L3 having an amino acid sequence of SEQ ID NO: 12.

E103. An isolated antibody that binds to human gastric inhibitory polypeptide receptor (GIPR), comprising a heavy chain and a light chain, wherein the heavy chain comprises a heavy chain variable region (VH) and the light chain comprises a light chain variable region (VL), and wherein (i) the VH comprises a complementary determining region (CDR)-H1 comprising an amino acid sequence of SEQ ID NO: 2, SEQ ID NO: 3, or SEQ ID NO: 4; a CDR-H2 comprising an amino acid sequence of SEQ ID NO: 5 or SEQ ID NO: 6; and a CDR-H3 comprising an amino acid sequence of SEQ ID NO: 7; and (ii) the VL comprises a CDR-L1 comprising an amino acid sequence of SEQ ID NO: 10; a CDR-L2 comprising an amino acid sequence of SEQ ID NO: 11; and a CDR-L3 comprising an amino acid sequence shown in SEQ ID NO: 12.

E104. The isolated antibody of any one of E1-E103, wherein the VH comprises an amino acid sequence of SEQ ID NO: 1, and the VL comprises an amino acid sequence of SEQ ID NO: 9.

E105. The antibody of any one of E1-104, wherein in the heavy chain comprises an amino acid sequence of SEQ ID NO: 8, and the light chain comprises an amino acid sequence of SEQ ID NO: 13.

E106. The antibody of any one of E1-E105, wherein the heavy chain comprises an amino acid sequence of SEQ ID NO: 8, and the light chain comprises an amino acid sequence of SEQ ID NO: 29.

E107. The antibody of any one of E1-E106, wherein the heavy chain comprises an amino acid sequence of SEQ ID NO: 27 or SEQ ID NO: 28, and the light chain comprises an amino acid sequence of SEQ ID NO: 13.

E108. The antibody of any one of E1-E107, wherein the (i) the heavy chain comprises an amino acid sequence of SEQ ID NO: 27, SEQ ID NO: 28, SEQ ID NO: 34, SEQ ID NO: 35, or SEQ ID NO: 36, and the light chain comprises an amino acid sequence of SEQ ID NO: 13; or (ii) the heavy chain comprises an amino acid sequence of SEQ ID NO: 8, and the light chain comprises an amino acid sequence of SEQ ID NO: 29.

E109. An isolated antibody comprising a light chain comprising an amino acid sequence of SEQ ID NO: 8; and a heavy chain comprising an amino acid sequence of SEQ ID NO: 13.

E110. An isolated antibody comprising a light chain comprising an amino acid sequence of SEQ ID NO: 21; and a heavy chain comprising an amino acid sequence of SEQ ID NO: 26.

E111. The antibody of any one of E1-E110, wherein the antibody has a binding affinity measured by surface plasmon resonance against a human GIPR extracellular domain that is less than about 15 nM.

E112. The antibody of any one of E1-E111, wherein the antibody has a binding affinity measured by surface plasmon resonance against a human GIPR extracellular domain that is less than about 10 nM.

E113. The antibody of any one of E1-E112, wherein the antibody has a binding affinity measured by surface plasmon resonance against a human GIPR extracellular domain that is less than about 5 nM.

E114. The antibody of any one of E1-E113, wherein the antibody has a half-maximal inhibitory concentration (IC₅₀) of less than about 28 nM in a human GIPR-CHO cAMP antagonist assay.

E115. The antibody of any one of E1-E114, wherein the antibody has a half-maximal inhibitory concentration (IC₅₀) of less than about 15 nM in a human GIPR-CHO cAMP antagonist assay.

E116. The antibody of any one of E1-E115, comprising one or both of a VH sequence encoded by a polynucleotide comprising the nucleic acid sequence of SEQ ID NO: 155 or SEQ ID NO: 159, and a VL sequence encoded by a polynucleotide comprising the nucleic acid sequence of SEQ ID NO: 157 or SEQ ID NO: 161.

E117. The antibody of any one of E1-E116, comprising one or both of a VH sequence encoded by a polynucleotide comprising the nucleic acid sequence of SEQ ID NO: 155, and a VL sequence encoded by a polynucleotide comprising the nucleic acid sequence of SEQ ID NO: 157.

E118. The antibody of any one of E1-E117, comprising one or both of a VH sequence encoded by a polynucleotide comprising the nucleic acid sequence of SEQ ID NO: 159, and a VL sequence encoded by a polynucleotide comprising the nucleic acid sequence of SEQ ID NO: 161.

E119. The antibody of any one of E1-E118, comprising one or both of a HC sequence encoded by a polynucleotide comprising the nucleic acid sequence of SEQ ID NO: 156 or SEQ ID NO: 160, and comprising a LC sequence encoded by a polynucleotide comprising the nucleic acid sequence of SEQ ID NO: 158 or SEQ ID NO: 162.

E120. The antibody of any one of E1-E119, comprising one or both of a HC sequence encoded by a polynucleotide comprising the nucleic acid sequence of SEQ ID NO: 156, and comprising a LC sequence encoded by a polynucleotide comprising the nucleic acid sequence of SEQ ID NO: 158.

E121. The antibody of any one of E1-E120, comprising one or both of a HC sequence encoded by a polynucleotide comprising the nucleic acid sequence of SEQ ID NO: 160, and comprising a LC sequence encoded by a polynucleotide comprising the nucleic acid sequence of SEQ ID NO: 162.

E122. A pharmaceutical composition comprising the antibody of any one of E1-E121; and a pharmaceutically acceptable carrier.

E123. An antibody conjugate comprising:
a) the antibody of any one of E1-E121, wherein the antibody comprises a conjugation site; and
b) a polypeptide conjugated to the antibody at the conjugation site through a linker, wherein:
(i) the conjugation site is a cysteine residue of the antibody heavy chain at position 118, 272, 290, 380 or 443, or a cysteine residue of the antibody light chain, at position 183 if the light chain is a Kappa light chain, and at position 205 if the light chain is a Lambda light chain, and the positions are based on EU numbering; and
(ii) the linker is selected from the group consisting of (GGGGS)₃, PEG12-K(AcBr)—NH₂ and PEG24-K(AcBr)—NH₂.

E124. The antibody conjugate of E123, wherein the polypeptide is a glucagon-like peptide-1 (GLP-1) receptor (GLP-1R) agonist, and wherein the GLP-1R agonist comprises an amino acid sequence that is a GLP-1 analog or an exendin 4 analog.

E125. The antibody conjugate of E123 or E124, wherein the polypeptide comprises an amino acid sequence selected from the group consisting of SEQ ID NO: 43, SEQ ID NO: 44, and SEQ ID NO: 47 to SEQ ID NO: 60.

E126. The antibody conjugate of E123, wherein the polypeptide is a glucagon receptor (GCGR) agonist, and the GCGR agonist comprises an amino acid sequence that is a glucagon analog.

E127. The antibody conjugate of E123, wherein the polypeptide is a dual agonist of GLP-1R and a GCGR, and wherein the polypeptide comprises an amino acid sequence that is both (i) a GLP-1 analog or a exendin 4 analog; and (ii) a GCG analog.

E128. The antibody conjugate of any one of E123-E127, wherein the polypeptide is a dual agonist of GLP-1R and GCGR, and wherein the polypeptide comprises an amino acid sequence that is both (i) 50% or more identical to the amino acid sequence of the wild type GLP-1 (7-37) or wild type exendin 4; and (ii) 50% or more identical to the amino acid sequence of the wildtype GCG.

E129. The antibody conjugate of any one of E123, E127, or E128, wherein the polypeptide comprises an amino acid sequence selected from the group consisting of SEQ ID NO: 73 to SEQ ID NO: 142.

E130. A pharmaceutical composition comprising the antibody conjugate of any one of E123-E129; and a pharmaceutically acceptable carrier.

E131. An isolated polynucleotide comprising a nucleic acid sequence that encodes the VH of the antibody of any one of E1-E121.

E132. An isolated polynucleotide comprising a nucleic acid sequence that encodes the VL of the antibody of any one of E1-E121.

E133. An isolated polynucleotide comprising (i) a nucleic acid sequence that encodes the VH and (ii) a nucleic acid sequence that encodes the VL, of the antibody of any one of E1-E121.

E134. The isolated polynucleotide of any one of E131-E133, wherein the polynucleotide is a ribonucleic acid.

E135. The isolated polynucleotide of any one of E131-E134, wherein the polynucleotide comprises a chemical modification.

E136. The isolated polynucleotide of E135, wherein the chemical modification is selected from the group consisting of: pseudouridine, 1-methylpseudouridine. N1-methylpseudouridine, N1-ethylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine), 5-methoxyuridine and 2′-O-methyl uridine.

E137. The isolated polynucleotide of any one of E131-E134, wherein the polynucleotide does not comprise a chemical modification.

E138. A vector comprising the polynucleotide of any one of embodiments E131-E137.

E139. An isolated host cell comprising the polynucleotide of any one of E131-E137.

E140. A method of producing an antibody comprising culturing the isolated host cell of E139 under conditions that result in production of an antibody and recovering the antibody.

E141. A method of treating a condition comprising administering to a subject in need thereof a therapeutically effective amount of the antibody of any one of E1-E121 or the antibody conjugate of any one of E123-E129.

E142. The method of E141, wherein the condition is a metabolic disorder.

E143. The method of E141, wherein the condition is selected from the group consisting of: diabetes, hyperglycemia, insulin resistance, hepatic insulin resistance, impaired glucose tolerance, diabetic neuropathy, diabetic nephropathy, acute kidney disorder, tubular dysfunction, proinflammatory changes to the proximal tubules, chronic kidney disease (CKD), diabetic retinopathy, adipocyte dysfunction, visceral adipose deposition, obstructive sleep apnea (OSA), obesity, hypothalamic obesity, monogenic obesity, obesity-related comorbidities, eating disorders, binge eating syndrome, bulimia nervosa, syndromic obesity, Prader-Willi syndrome, Bardet-Biedl syndrome, weight gain, being overweight, excessive sugar cravings, dyslipidemia, hyperlipidemia, hypertriglyceridemia, increased total cholesterol, high LDL (low-density lipoprotein) cholesterol, low HDL (high-density lipoprotein) cholesterol, hyperinsulinemia, nonalcoholic fatty liver disease, steatosis, nonalcoholic steatohepatitis (NASH), fibrosis, cirrhosis, and hepatocellular carcinoma, cardiovascular disease, atherosclerosis, coronary artery disease, peripheral vascular disease, hypertension, endothelial dysfunction, impaired vascular compliance, heart failure, congestive heart failure, heart failure with preserved ejection fraction (HFpEF), heart failure with reduced ejection fraction (HFrEF), myocardial infarction, stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease (PAD), macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, polycystic ovary syndrome (PCOS), and addiction.

E144. The method of E141, wherein the condition is obesity.

E145. The method of E141, wherein the condition is diabetes.

E146. The method of E145, wherein the diabetes is selected from the group consisting of: type 1 diabetes, type 2 diabetes, pre-diabetes, idiopathic type 1b diabetes, latent autoimmune diabetes in adults (LADA), early-onset type 2 diabetes (EOD), youth-onset atypical diabetes (YOAD), maturity onset diabetes of the young (MODY), malnutrition-related diabetes, and gestational diabetes.

E147. The method of E141, wherein the condition is elevated blood sugar levels and/or hemoglobin A1c levels.

E148. A method of comprising administering to a subject in need thereof a therapeutically effective amount of the antibody of any one of E1-E121 or the antibody conjugate of any one of E123-E129, wherein the administering reduces a body mass index (BMI) of the subject by at least about 5%.

E149. The method of any one of E141-E148, wherein the administering is subcutaneous.

E150. The method of any one of E141-E149, wherein the administering is with an injection pen device.

E151. The method of any one of E141-E150, wherein the administering is once a week.

E152. The method of any one of E141-E150, wherein the administering is once a month.

E153. The method of any one of E141-E150, wherein the administering is twice a month.

E154. The method of any one of E141-E153, wherein the administering is in conjunction with the subject having a healthy diet and exercise regimen.

E155. The method of E154, wherein the healthy diet comprises the subject eating fewer calories than an estimated total daily energy expenditure of the subject.

E156. The method of E154, wherein the exercise regimen comprises the subject exercising at least about 150 minutes of moderate-intensity physical activity per week or about 75 minutes of vigorous-intensity activity.

E157. The method of any one of E141-E156, wherein the subject is human.

E158. The method of any one of E141-E157, wherein the subject has an initial body mass index (BMI) of at least about 27 kg/m².

E159. The method of any one of E141-E158, wherein the subject has an initial body mass index (BMI) of at least about 30 kg/m².

E160. The method of any one of E141-E159, wherein the subject further comprises at least one weight-related comorbid condition.

E161. The method of any one of E141-E160, wherein the therapeutically effective amount is from about 1 mg to about 250 mg.

E162. The method of any one of E141-E161, wherein the therapeutically effective amount is from about 50 mg to about 150 mg.

E163. The antibody of any one of E1-E121 or the antibody conjugate of any one of E123-E129, for use as a medicament.

E164. The antibody of any one of E1-E121 or the antibody conjugate of any one of E123-E129, for use in the treatment of a condition disclosed herein.

E165. The use of the antibody of any one of E1-E121 or the antibody conjugate of any one of E123-E129 for the manufacture of a medicament for use in the treatment of a condition disclosed herein.

E166. A pharmaceutical composition for the treatment of a condition disclosed herein, comprising the antibody of any one of E1-E121 or the antibody conjugate of any one of E123-E129.

E167. An anti-disease agent comprising the antibody of any one of E1-E121 or the antibody conjugate of any one of E123-E129.

E168. An isolated peptide comprising an amino acid sequence selected from the group consisting of SEQ ID NO: 43, SEQ ID NO: 44, and SEQ ID NO: 47 to SEQ ID NO: 60.

E169. An isolated peptide having an amino acid sequence selected from the group consisting of SEQ ID NO: 43, SEQ ID NO: 44, and SEQ ID NO: 47 to SEQ ID NO: 60.

E170. An isolated polypeptide comprising an amino acid sequence selected from the group consisting of SEQ ID NO: 73 to SEQ ID NO: 142.

E171. An isolated polypeptide having an amino acid sequence selected from the group consisting of SEQ ID NO: 73 to SEQ ID NO: 142.

What is claimed is:

1. An isolated antibody that binds to human gastric inhibitory polypeptide receptor (GIPR), comprising a heavy chain and a light chain, and the heavy chain comprises a heavy chain variable region (VH) and the light chain comprises a light chain variable region (VL), wherein
(i) the VH comprises: a complementary determining region (CDR)-H1 comprising an amino acid sequence of SEQ ID NO: 15, SEQ ID NO: 16, or SEQ ID NO: 17; a VH CDR-H2 comprising an amino acid sequence of SEQ ID NO: 18 or SEQ ID NO: 19; and a VH CDR-H3 comprising an amino acid sequence of SEQ ID NO: 20; and
(ii) the VL comprises” a CDR-L1 comprising an amino acid sequence of SEQ ID NO: 23; a VL CDR-L2 comprising an amino acid sequence of SEQ ID NO: 24; and a VL CDR-L3 comprising an amino acid sequence of SEQ ID NO: 25.

2. The isolated antibody of claim 1, wherein the VH comprises an amino acid sequence of SEQ ID NO: 14, and the VL comprises an amino acid sequence of SEQ ID NO: 22.

3. The isolated antibody of claim 1 or 2, where in the heavy chain comprises an amino acid sequence of SEQ ID NO: 21, and the light chain comprises an amino acid sequence of SEQ ID NO: 26.

4. An isolated antibody that binds to human gastric inhibitory polypeptide receptor (GIPR), comprising a heavy chain and a light chain, wherein the heavy chain comprises a heavy chain variable region (VH) and the light chain comprises a light chain variable region (VL), and wherein
(i) the VH comprises a VH complementary determining region (CDR)-H1 comprising an amino acid sequence of SEQ ID NO: 2, SEQ ID NO: 3, or SEQ ID NO: 4; a VH CDR-H2 comprising an amino acid sequence of SEQ ID NO: 5 or SEQ ID NO: 6; and a VH CDR-H3 comprising an amino acid sequence of SEQ ID NO: 7; and
(ii) the VL comprises a VL CDR-L1 comprising an amino acid sequence of SEQ ID NO: 10; a VL CDR-L2 comprising an amino acid sequence of SEQ ID NO: 11; and a VL CDR-L3 comprising an amino acid sequence shown in SEQ ID NO: 12.

5. The isolated antibody of claim 4, wherein the VH comprises an amino acid sequence of SEQ ID NO: 1, and the VL comprises an amino acid sequence of SEQ ID NO: 9.

6. The isolated antibody of claim 4 or 5, where in the heavy chain comprises an amino acid sequence of SEQ ID NO: 8, and the light chain comprises an amino acid sequence of SEQ ID NO: 13.

7. The isolated antibody of any one of claims 1 to 6, wherein the isolated antibody is a full-length human or humanized IgG antibody comprising two heavy chains and two light chains, wherein the two heavy chains each have a first pair of identical amino acid sequences, and the two light chains each have a second pair of identical amino acid sequences.

8. An isolated antibody comprising a light chain comprising an amino acid sequence of SEQ ID NO: 8; and a heavy chain comprising an amino acid sequence of SEQ ID NO: 13.

9. An isolated antibody comprising a light chain comprising an amino acid sequence of SEQ ID NO: 21; and a heavy chain comprising an amino acid sequence of SEQ ID NO: 26.

10. A pharmaceutical composition comprising the isolated antibody of any one of claims 1 to 9; and a pharmaceutically acceptable carrier.

11. An isolated polynucleotide comprising a nucleic acid sequence that encodes the VH of the isolated antibody of any one of claims 1 to 9.

12. A vector comprising the isolated polynucleotide of claim 11.

13. An isolated host cell comprising the isolated polynucleotide of claim 11.

14. A method of producing an antibody comprising culturing the isolated host cell of claim 19 under conditions that result in production of an antibody and recovering the antibody.

15. A method of treating a condition comprising administering to a subject in need thereof a therapeutically effective amount of an isolated antibody of any one of claims 1 to 9.

16. The method of claim 15, wherein the condition is selected from the group consisting of: diabetes, hyperglycemia, insulin resistance, hepatic insulin resistance, impaired glucose tolerance, diabetic neuropathy, diabetic nephropathy, acute kidney disorder, tubular dysfunction, proinflammatory changes to the proximal tubules, chronic kidney disease (CKD), diabetic retinopathy, adipocyte dysfunction, visceral adipose deposition, obstructive sleep apnea (OSA), obesity, hypothalamic obesity, monogenic obesity, obesity-related comorbidities, eating disorders, binge eating syndrome, bulimia nervosa, syndromic obesity, Prader-Willi syndrome, Bardet-Biedl syndrome, weight gain, being overweight, excessive sugar cravings, dyslipidemia, hyperlipidemia, hypertriglyceridemia, increased total cholesterol, high LDL (low-density lipoprotein) cholesterol, low HDL (high-density lipoprotein) cholesterol, hyperinsulinemia, nonalcoholic fatty liver disease, steatosis, nonalcoholic steatohepatitis (NASH), fibrosis, cirrhosis, and hepatocellular carcinoma, cardiovascular disease, atherosclerosis, coronary artery disease, peripheral vascular disease, hypertension, endothelial dysfunction, impaired vascular compliance, heart failure, congestive heart failure, heart failure with preserved ejection fraction (HFpEF), heart failure with reduced ejection fraction (HFrEF), myocardial infarction, stroke, hemorrhagic stroke, ischemic stroke, traumatic brain injury, pulmonary hypertension, restenosis after angioplasty, intermittent claudication, post-prandial lipemia, metabolic acidosis, ketosis, arthritis, osteoporosis, osteoarthritis, Parkinson's disease, left ventricular hypertrophy, peripheral arterial disease (PAD), macular degeneration, cataract, glomerulosclerosis, chronic renal failure, metabolic syndrome, syndrome X, premenstrual syndrome, angina pectoris, thrombosis, atherosclerosis, transient ischemic attacks, vascular restenosis, impaired glucose metabolism, conditions of impaired fasting plasma glucose, hyperuricemia, gout, erectile dysfunction, skin and connective tissue disorders, psoriasis, foot ulcerations, ulcerative colitis, hyper apo B lipoproteinemia, Alzheimer's Disease, schizophrenia, impaired cognition, inflammatory bowel disease, short bowel syndrome, Crohn's disease, colitis, irritable bowel syndrome, polycystic ovary syndrome (PCOS), and addiction.


SEQUENCE LISTING (162 sequences)

SEQ ID NO: 1 - protein, 120 aa, synthetic construct
QVQLVQSGAEVKKPGSSVKVSCKASGYTFSSYAINWVRQAPGQGLEWMGGIVPIFGIANYAQKFQGRVTITADESTSTAYMELSSLRSEDTAVYYCARGSDYVVPHFDYWGQGTLVTVSS

SEQ ID NO: 2 - protein, 7 aa, synthetic construct
GYTFSSY

SEQ ID NO: 3 - protein, 5 aa, synthetic construct
SYAIN

SEQ ID NO: 4 - protein, 10 aa, synthetic construct
GYTFSSYAIN

SEQ ID NO: 5 - protein, 8 aa, synthetic construct
GIVPIFGI

SEQ ID NO: 6 - protein, 17 aa, synthetic construct
GIVPIFGIANYAQKFQG

SEQ ID NO: 7 - protein, 11 aa, synthetic construct
GSDYVVPHFDY

SEQ ID NO: 8 - protein, 450 aa, synthetic construct
QVQLVQSGAEVKKPGSSVKVSCKASGYTFSSYAINWVRQAPGQGLEWMGGIVPIFGIANYAQKFQGRVTITADESTSTAYMELSSLRSEDTAVYYCARGSDYVVPHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 110 aa, synthetic construct
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGVVFGGGTKLTVL

SEQ ID NO: 10 - protein, 13 aa, synthetic construct
SGSSSNIGSNYVY

SEQ ID NO: 11 - protein, 7 aa, synthetic construct
RNNQRPS

SEQ ID NO: 12 - protein, 11 aa, synthetic construct
AAWDDSLSGVV

SEQ ID NO: 13 - protein, 216 aa, synthetic construct
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 14 - protein, 128 aa, synthetic construct
EVQLVESGGGLVKPGGSLRLSCAASGFPFSSAWMSWVRQAPGKGLEWVGRIKSKADAGTTDYGAPVKGRFTISRDDSKNTLYLQMNSLKTEDTAVYYCTTEDIVGVPFKGYYQYMDAWGKGTTVTVSS

SEQ ID NO: 15 - protein, 7 aa, synthetic construct
GFPFSSA

SEQ ID NO: 16 - protein, 5 aa, synthetic construct
SAWMS

SEQ ID NO: 17 - protein, 10 aa, synthetic construct
GFPFSSAWMS

SEQ ID NO: 18 - protein, 10 aa, synthetic construct
RIKSKADAGT

SEQ ID NO: 19 - protein, 19 aa, synthetic construct
RIKSKADAGTTDYGAPVKG

SEQ ID NO: 20 - protein, 17 aa, synthetic construct
EDIVGVPFKGYYQYMDA

SEQ ID NO: 21 - protein, 458 aa, synthetic construct
EVQLVESGGGLVKPGGSLRLSCAASGFPFSSAWMSWVRQAPGKGLEWVGRIKSKADAGTTDYGAPVKGRFTISRDDSKNTLYLQMNSLKTEDTAVYYCTTEDIVGVPFKGYYQYMDAWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 108 aa, synthetic construct
EDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIK

SEQ ID NO: 23 - protein, 11 aa, synthetic construct
RASQSISSYLN

SEQ ID NO: 24 - protein, 7 aa, synthetic construct
AASSLQS

SEQ ID NO: 25 - protein, 9 aa, synthetic construct
QQSYSTPLT

SEQ ID NO: 26 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 450 aa, synthetic construct
QVQLVQSGAEVKKPGSSVKVSCKASGYTFSSYAINWVRQAPGQGLEWMGGIVPIFGIANYAQKFQGRVTITADESTSTAYMELSSLRSEDTAVYYCARGSDYVVPHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTCPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 450 aa, synthetic construct
QVQLVQSGAEVKKPGSSVKVSCKASGYTFSSYAINWVRQAPGQGLEWMGGIVPIFGIANYAQKFQGRVTITADESTSTAYMELSSLRSEDTAVYYCARGSDYVVPHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPCVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 216 aa, synthetic construct
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTCEKTVAPTECS

SEQ ID NO: 30 - protein, 458 aa, synthetic construct
EVQLVESGGGLVKPGGSLRLSCAASGFPFSSAWMSWVRQAPGKGLEWVGRIKSKADAGTTDYGAPVKGRFTISRDDSKNTLYLQMNSLKTEDTAVYYCTTEDIVGVPFKGYYQYMDAWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTCPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 214 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKRTVAAPSVFIFPPSDEQLKSGTASVVCLLNNFYPREAKVQWKVDNALQSGNSQESVTEQDSKDSTYSLSSTLTLSCADYEKHKVYACEVTHQGLSSPVTKSFNRGEC

SEQ ID NO: 32 - protein, 458 aa, synthetic construct
EVQLVESGGGLVKPGGSLRLSCAASGFPFSSAWMSWVRQAPGKGLEWVGRIKSKADAGTTDYGAPVKGRFTISRDDSKNTLYLQMNSLKTEDTAVYYCTTEDIVGVPFKGYYQYMDAWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPCVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 148 aa, synthetic construct
RAETGSKGQTAGELYQRWERYRRECQETLAAAEPPSGLACNGSFDMYVCWDYAAPNATARASCPWYLPWHHHVAAGFVLRQCGSDGQWGLWRDHTQCENPEKNEAFLDQRLILERLQLEVLFQGPGLNDIFEAQKIEWHEGSENLYFQ

SEQ ID NO: 34 - protein, 450 aa, synthetic construct
QVQLVQSGAEVKKPGSSVKVSCKASGYTFSSYAINWVRQAPGQGLEWMGGIVPIFGIANYAQKFQGRVTITADESTSTAYMELSSLRSEDTAVYYCARGSDYVVPHFDYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 450 aa, synthetic construct
QVQLVQSGAEVKKPGSSVKVSCKASGYTFSSYAINWVRQAPGQGLEWMGGIVPIFGIANYAQKFQGRVTITADESTSTAYMELSSLRSEDTAVYYCARGSDYVVPHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVCWESNGQPENNYKTTPPVLDSDGSFFLYSKLTVDKSRWQQGNVFSCSVMHEALHNHYTQKSLSLSPGK

SEQ ID NO: 36 - protein, 450 aa, synthetic construct
QVQLVQSGAEVKKPGSSVKVSCKASGYTFSSYAINWVRQAPGQGLEWMGGIVPIFGIANYAQKFQGRVTITADESTSTAYMELSSLRSEDTAVYYCARGSDYVVPHFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 37 - protein, 458 aa, synthetic construct
EVQLVESGGGLVKPGGSLRLSCAASGFPFSSAWMSWVRQAPGKGLEWVGRIKSKADAGTTDYGAPVKGRFTISRDDSKNTLYLQMNSLKTEDTAVYYCTTEDIVGVPFKGYYQYMDAWGKGTTVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 458 aa, synthetic construct
EVQLVESGGGLVKPGGSLRLSCAASGFPFSSAWMSWVRQAPGKGLEWVGRIKSKADAGTTDYGAPVKGRFTISRDDSKNTLYLQMNSLKTEDTAVYYCTTEDIVGVPFKGYYQYMDAWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVCWESNGQPENNYKTTPPVLDSDGSFFLYSKLTVDKSRWQQGNVFSCSVMHEALHNHYTQKSLSLSPGK

SEQ ID NO: 39 - protein, 458 aa, synthetic construct
EVQLVESGGGLVKPGGSLRLSCAASGFPFSSAWMSWVRQAPGKGLEWVGRIKSKADAGTTDYGAPVKGRFTISRDDSKNTLYLQMNSLKTEDTAVYYCTTEDIVGVPFKGYYQYMDAWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSCSPGK

SEQ ID NO: 40 - protein, 31 aa, synthetic construct
HAEGTFTSDVSSYLEGQAAKEFIAWLVKGRG

SEQ ID NO: 41 - protein, 31 aa, synthetic construct; identical to SEQ ID NO: 40

SEQ ID NO: 42 - protein, 31 aa, synthetic construct; identical to SEQ ID NO: 40

SEQ ID NO: 43 - protein, 31 aa, synthetic construct
  MOD_RES 2: note = Aib
HXEGTFTSDYSSYLEEQAAKEFIAWLVKGRG

SEQ ID NO: 44 - protein, 31 aa, synthetic construct
  MOD_RES 2: note = Aib
  MOD_RES 16: note = Aib
HXEGTFTSDYSSYLEXQAAKEFIAWLVKGRG

SEQ ID NO: 45 - protein, 31 aa, synthetic construct
  MOD_RES 2: note = Aib
  MOD_RES 16: note = Aib
HXEGTFTSDVSSYLEXQAAKEFIAWLVKGRG

SEQ ID NO: 46 - protein, 31 aa, synthetic construct
  MOD_RES 2: note = Aib
HXEGTFTSDVSSYLEEQAAKEFIAWLVKGRG

SEQ ID NO: 47 - protein, 29 aa, synthetic construct
  MOD_RES 2: note = Aib
HXEGTFTSDYSSYLEEQAAKEFIAWLVKG

SEQ ID NO: 48 - protein, 29 aa, synthetic construct
  MOD_RES 2: note = Aib
  MOD_RES 16: note = Aib
HXEGTFTSDYSSYLEXQAAKEFIAWLVKG

SEQ ID NO: 49 - protein, 30 aa, synthetic construct
  MOD_RES 2: note = Aib
HXEGTFTSDYSSYLEEQAAKEFIAWLVKGG

SEQ ID NO: 50 - protein, 30 aa, synthetic construct
  MOD_RES 2: note = Aib
  MOD_RES 16: note = Aib
HXEGTFTSDYSSYLEXQAAKEFIAWLVKGG

SEQ ID NO: 51 - protein, 31 aa, synthetic construct
  MOD_RES 2: note = Aib
HXEGTFTSDYSSYLEEQAAKEFIAWLVRGGG

SEQ ID NO: 52 - protein, 31 aa, synthetic construct
  MOD_RES 2: note = Aib
  MOD_RES 16: note = Aib
HXEGTFTSDYSSYLEXQAAKEFIAWLVRGGG

SEQ ID NO: 53 - protein, 31 aa, synthetic construct
  MOD_RES 2: note = Aib
HXEGTFTSDYSSYLEEQAAKEFIAWLVRGRG

SEQ ID NO: 54 - protein, 31 aa, synthetic construct
  MOD_RES 2: note = Aib
  MOD_RES 16: note = Aib
HXEGTFTSDYSSYLEXQAAKEFIAWLVRGRG

SEQ ID NO: 55 - protein, 31 aa, synthetic construct
HGEGTFTSDYSSYLEEQAAKEFIAWLVKGGG

SEQ ID NO: 56 - protein, 31 aa, synthetic construct
  MOD_RES 16: note = Aib
HGEGTFTSDYSSYLEXQAAKEFIAWLVKGGG

SEQ ID NO: 57 - protein, 31 aa, synthetic construct
  MOD_RES 2: note = Aib
HXEGTFTSDYSSYLEAQAAKEFIAWLVKGGG

SEQ ID NO: 58 - protein, 31 aa, synthetic construct
  MOD_RES 2: note = Aib
HXEGTFTSDYSSYLEEQAAKEFIAWLVKGGG

SEQ ID NO: 59 - protein, 31 aa, synthetic construct
  MOD_RES 2: note = Aib
  MOD_RES 16: note = Aib
HXEGTFTSDVSSYLEXQAAKEFIAWLVKGGG

SEQ ID NO: 60 - protein, 29 aa, synthetic construct
  MOD_RES 2: note = Aib
  MOD_RES 16: note = Aib
HXEGTFTSDVSSYLEXQAAKEFIAWLVKG

SEQ ID NO: 61 - protein, 39 aa, synthetic construct
HGEGTFTSDLSKQMEEEAVRLFIEWLKNGGPSSGAPPPS

SEQ ID NO: 62 - protein, 29 aa, synthetic construct
HSQGTGTSDYSKYLDSRRAQDFVQWLMNT

SEQ ID NO: 63 - protein, 34 aa, synthetic construct
  MOD_RES 2: note = Aib
HXQGTFTSDYSKYLDEKKAKEFVEWLLEGGPSSG

SEQ ID NO: 64 - protein, 29 aa, synthetic construct
  MOD_RES 2: note = 1-amino-1-cyclobutanecarboxylic acid
HXQGTFTSDYSKYLDERAAKDFIKWLESA

SEQ ID NO: 65 - protein, 29 aa, synthetic construct
  MOD_RES 2: note = Aib
HXQGTFTSDYSKYLDEKAAKEFIQWLLQT

SEQ ID NO: 66 - protein, 29 aa, synthetic construct
  MOD_RES 2: note = Aib
HXQGTFTSDLSKYLESKRAREFVQWLLKT

SEQ ID NO: 67 - protein, 29 aa, synthetic construct
  MOD_RES 2: note = 1-amino-1-cyclobutanecarboxylic acid
HXQGTFTSDLSKYLDLRRARDFVQWLLKT

SEQ ID NO: 68 - protein, 29 aa, synthetic construct
  MOD_RES 1: note = deamino-histidine
  MOD_RES 2: note = Aib
XXHGTFTSDVSKYLEEQAAREFIAWLLKT

SEQ ID NO: 69 - protein, 40 aa, synthetic construct
  MOD_RES 2: note = Aib
  MOD_RES 29: note = Aib
  MOD_RES 38: note = 4-(((1S)-2-amino-1-((2,5-dioxo-1-(13-oxo-2,5,8-trio xa-12-azatetradecan-14-yl)pyrrolidin-3-yl)thio)-2-oxoethyl )amino)-1-(2,5-dioxo-1-(13-oxo-2,5,8-trioxa-12-azapentadec an-15-yl)pyrrolidin-3-yl)-4-oxobutan-2-ide
HXQGTFTSDYSKYLDSKKAQEFVEWLLNXGRNRNNIANHX

SEQ ID NO: 70 - protein, 29 aa, synthetic construct
HSQGTFTSDYSKYLDEQAAKEFIAWLMNT

SEQ ID NO: 71 - protein, 41 aa, synthetic construct
  MOD_RES 2: note = Aib
  MOD_RES 13: note = alpha methyl leucine
  MOD_RES 20: note = Aib
YXQGTFTSDYSIXLDKKAQXAFIEYLLEGGPSSGAPPPSNH

SEQ ID NO: 72 - protein, 29 aa, synthetic construct
  MOD_RES 2: note = d-Serine
HXQGTFTSDKSKYLDARAAQDFVQWLLDT

SEQ ID NO: 73 - protein, 34 aa, synthetic construct
  MOD_RES 1: note = deamino-histidine
  MOD_RES 2: note = Aib
XXQGTFTSDYSKYLDEKKAKEFVEWLLEGGPSSG

SEQ ID NO: 74 - protein, 34 aa, synthetic construct; identical to SEQ ID NO: 73
  MOD_RES 1: note = 3-(1H-Benzimidazol-5-yl)propanoic acid
  MOD_RES 2: note = Aib

SEQ ID NO: 75 - protein, 34 aa, synthetic construct
  MOD_RES 2: note = Aib
FXQGTFTSDYSKYLDEKKAKEFVEWLLEGGPSSG

SEQ ID NO: 76 - protein, 34 aa, synthetic construct
  MOD_RES 2: note = Aib
YXQGTFTSDYSKYLDEKKAKEFVEWLLEGGPSSG

SEQ ID NO: 77 - protein, 34 aa, synthetic construct; identical to SEQ ID NO: 73
  MOD_RES 1: note = 3-(1H-Pyrazol-4-yl)propanoic acid
  MOD_RES 2: note = Aib

SEQ ID NO: 78 - protein, 34 aa, synthetic construct; identical to SEQ ID NO: 73
  MOD_RES 1: note = 3-(1H-Pyrazol-4-yl)-L-alanine
  MOD_RES 2: note = Aib

SEQ ID NO: 79 - protein, 34 aa, synthetic construct; identical to SEQ ID NO: 73
  MOD_RES 1: note = 3-(1H-Benzimidazole-5-yl)-L-alanine
  MOD_RES 2: note = Aib

SEQ ID NO: 80 - protein, 34 aa, synthetic construct
HAQGTFTSDYSKYLDEKKAKEFVEWLLEGGPSSG

SEQ ID NO: 81 - protein, 34 aa, synthetic construct
HGQGTFTSDYSKYLDEKKAKEFVEWLLEGGPSSG

SEQ ID NO: 82 - protein, 34 aa, synthetic construct
HSQGTFTSDYSKYLDEKKAKEFVEWLLEGGPSSG

SEQ ID NO: 83 - protein, 34 aa, synthetic construct; identical to SEQ ID NO: 63
  MOD_RES 2: note = 1-amino-1-cyclobutanecarboxylic acid

SEQ ID NO: 84 - protein, 34 aa, synthetic construct; identical to SEQ ID NO: 63
  MOD_RES 2: note = 2-Methyl-L-proline

SEQ ID NO: 85 - protein, 34 aa, synthetic construct; identical to SEQ ID NO: 63
  MOD_RES 2: note = d-serine

SEQ ID NO: 86 - protein, 34 aa, synthetic construct; identical to SEQ ID NO: 63
  MOD_RES 2: note = d-alanine

SEQ ID NO: 87 - protein, 34 aa, synthetic construct
  MOD_RES 2: note = Aib
HXEGTFTSDYSKYLDEKKAKEFVEWLLEGGPSSG

SEQ ID NO: 88 - protein, 34 aa, synthetic construct
  MOD_RES 2: note = Aib
HXHGTFTSDYSKYLDEKKAKEFVEWLLEGGPSSG

SEQ ID NO: 89 - protein, 34 aa, synthetic construct
  MOD_RES 2: note = Aib
  MOD_RES 4: note = d-alanine
HXQXTFTSDYSKYLDEKKAKEFVEWLLEGGPSSG

SEQ ID NO: 90 - protein, 34 aa, synthetic construct
  MOD_RES 2: note = Aib
  MOD_RES 6: note = alpha methyl L-phenylalanine
HXQGTXTSDYSKYLDEKKAKEFVEWLLEGGPSSG

SEQ ID NO: 91 - protein, 34 aa, synthetic construct
  MOD_RES 2: note = Aib
HXQGTFTSDVSKYLDEKKAKEFVEWLLEGGPSSG

SEQ ID NO: 92 - protein, 34 aa, synthetic construct
  MOD_RES 2: note = Aib
HXQGTFTSDKSKYLDEKKAKEFVEWLLEGGPSSG

SEQ ID NO: 93 - protein, 34 aa, synthetic construct
  MOD_RES 2: note = Aib
HXQGTFTSDLSKYLDEKKAKEFVEWLLEGGPSSG

SEQ ID NO: 94 - protein, 34 aa, synthetic construct
  MOD_RES 2: note = Aib
HXQGTFTSDASKYLDEKKAKEFVEWLLEGGPSSG

SEQ ID NO: 95 - protein, 34 aa, synthetic construct
  MOD_RES 2: note = Aib
  MOD_RES 11: note = 5-Phenyl-L-norvaline
HXQGTFTSDYXKYLDEKKAKEFVEWLLEGGPSSG

SEQ ID NO: 96 - protein, 34 aa, synthetic construct
  MOD_RES 2: note = Aib
HXQGTFTSDYAKYLDEKKAKEFVEWLLEGGPSSG

SEQ ID NO: 97 - protein, 34 aa, synthetic construct
  MOD_RES 2: note = Aib
HXQGTFTSDYSSYLDEKKAKEFVEWLLEGGPSSG

SEQ ID NO: 98 - protein, 34 aa, synthetic construct
  MOD_RES 2: note = Aib
HXQGTFTSDYSEYLDEKKAKEFVEWLLEGGPSSG

SEQ ID NO: 99 - protein, 34 aa, synthetic construct
  MOD_RES 2: note = Aib
HXQGTFTSDYSIYLDEKKAKEFVEWLLEGGPSSG

SEQ ID NO: 100 - protein, 34 aa, synthetic construct
  MOD_RES 2: note = Aib
HXQGTFTSDYSAYLDEKKAKEFVEWLLEGGPSSG

SEQ ID NO: 101 - protein, 34 aa, synthetic construct
  MOD_RES 2: note = Aib
HXQGTFTSDYSKQLDEKKAKEFVEWLLEGGPSSG

SEQ ID NO: 102 - protein, 34 aa, synthetic construct
  MOD_RES 2: note = Aib
  MOD_RES 13: note = alpha methyl L-2-flurophenylalanine
HXQGTFTSDYSKXLDEKKAKEFVEWLLEGGPSSG

SEQ ID NO: 103 - protein, 34 aa, synthetic construct; identical to SEQ ID NO: 102
  MOD_RES 2: note = Aib
  MOD_RES 13: note = alpha methyl L-Leucine

SEQ ID NO: 104 - protein, 34 aa, synthetic construct
  MOD_RES 2: note = Aib
HXQGTFTSDYSKYMDEKKAKEFVEWLLEGGPSSG

SEQ ID NO: 105 - protein, 34 aa, synthetic construct
  MOD_RES 2: note = Aib
HXQGTFTSDYSKYLEEKKAKEFVEWLLEGGPSSG

SEQ ID NO: 106 - protein, 34 aa, synthetic construct
  MOD_RES 2: note = Aib
HXQGTFTSDYSKYLDGKKAKEFVEWLLEGGPSSG

SEQ ID NO: 107 - protein, 34 aa, synthetic construct
  MOD_RES 2: note = Aib
HXQGTFTSDYSKYLDSKKAKEFVEWLLEGGPSSG

SEQ ID NO: 108 - protein, 34 aa, synthetic construct
  MOD_RES 2: note = Aib
  MOD_RES 16: note = Aib
HXQGTFTSDYSKYLDXKKAKEFVEWLLEGGPSSG

SEQ ID NO: 109 - protein, 34 aa, synthetic construct
  MOD_RES 2: note = Aib
HXQGTFTSDYSKYLDAKKAKEFVEWLLEGGPSSG

SEQ ID NO: 110 - protein, 34 aa, synthetic construct; identical to SEQ ID NO: 108
  MOD_RES 2: note = Aib
  MOD_RES 16: note = 2-Methyl-L-glutamic acid

SEQ ID NO: 111 - protein, 34 aa, synthetic construct
  MOD_RES 2: note = Aib
HXQGTFTSDYSKYLDEQKAKEFVEWLLEGGPSSG

SEQ ID NO: 112 - protein, 34 aa, synthetic construct
  MOD_RES 2: note = Aib
HXQGTFTSDYSKYLDERKAKEFVEWLLEGGPSSG

SEQ ID NO: 113 - protein, 34 aa, synthetic construct
  MOD_RES 2: note = Aib
HXQGTFTSDYSKYLDEEKAKEFVEWLLEGGPSSG

SEQ ID NO: 114 - protein, 34 aa, synthetic construct
  MOD_RES 2: note = Aib
HXQGTFTSDYSKYLDEAKAKEFVEWLLEGGPSSG

SEQ ID NO: 115 - protein, 34 aa, synthetic construct
  MOD_RES 2: note = Aib
HXQGTFTSDYSKYLDEKAAKEFVEWLLEGGPSSG

SEQ ID NO: 116 - protein, 34 aa, synthetic construct
  MOD_RES 2: note = Aib
HXQGTFTSDYSKYLDEKRAKEFVEWLLEGGPSSG

SEQ ID NO: 117 - protein, 34 aa, synthetic construct
  MOD_RES 2: note = Aib
HXQGTFTSDYSKYLDEKSAKEFVEWLLEGGPSSG

SEQ ID NO: 118 - protein, 34 aa, synthetic construct
  MOD_RES 2: note = Aib
HXQGTFTSDYSKYLDEKKVKEFVEWLLEGGPSSG

SEQ ID NO: 119 - protein, 34 aa, synthetic construct
  MOD_RES 2: note = Aib
HXQGTFTSDYSKYLDEKKLKEFVEWLLEGGPSSG

SEQ ID NO: 120 - protein, 34 aa, synthetic construct
  MOD_RES 2: note = Aib
  MOD_RES 19: note = Aib
HXQGTFTSDYSKYLDEKKXKEFVEWLLEGGPSSG

SEQ ID NO: 121 - protein, 34 aa, synthetic construct
  MOD_RES 2: note = Aib
HXQGTFTSDYSKYLDEKKAREFVEWLLEGGPSSG

SEQ ID NO: 122 - protein, 34 aa, synthetic construct
  MOD_RES 2: note = Aib
HXQGTFTSDYSKYLDEKKAQEFVEWLLEGGPSSG

SEQ ID NO: 123 - protein, 34 aa, synthetic construct
  MOD_RES 2: note = Aib
HXQGTFTSDYSKYLDEKKANEFVEWLLEGGPSSG

SEQ ID NO: 124 - protein, 34 aa, synthetic construct
  MOD_RES 2: note = Aib
  MOD_RES 20: note = Aib
HXQGTFTSDYSKYLDEKKAXEFVEWLLEGGPSSG

SEQ ID NO: 125 - protein, 34 aa, synthetic construct
  MOD_RES 2: note = Aib
HXQGTFTSDYSKYLDEKKAKDFVEWLLEGGPSSG

SEQ ID NO: 126 - protein, 34 aa, synthetic construct
  MOD_RES 2: note = Aib
HXQGTFTSDYSKYLDEKKAKQFVEWLLEGGPSSG

SEQ ID NO: 127 - protein, 34 aa, synthetic construct; identical to SEQ ID NO: 126
  MOD_RES 2: note = Aib

SEQ ID NO: 128 - protein, 34 aa, synthetic construct
  MOD_RES 2: note = Aib
HXQGTFTSDYSKYLDEKKAKEFIEWLLEGGPSSG

SEQ ID NO: 129 - protein, 34 aa, synthetic construct
  MOD_RES 2: note = Aib
HXQGTFTSDYSKYLDEKKAKEFAEWLLEGGPSSG

SEQ ID NO: 130 - protein, 34 aa, synthetic construct
  MOD_RES 2: note = Aib
HXQGTFTSDYSKYLDEKKAKEFVAWLLEGGPSSG

SEQ ID NO: 131 - protein, 34 aa, synthetic construct
  MOD_RES 2: note = Aib
HXQGTFTSDYSKYLDEKKAKEFVKWLLEGGPSSG

SEQ ID NO: 132 - protein, 34 aa, synthetic construct
  MOD_RES 2: note = Aib
HXQGTFTSDYSKYLDEKKAKEFVQWLLEGGPSSG

SEQ ID NO: 133 - protein, 29 aa, synthetic construct
  MOD_RES 2: note = Aib
HXQGTFTSDYSKYLDEKKAKEFVEWLMNT

SEQ ID NO: 134 - protein, 29 aa, synthetic construct
  MOD_RES 2: note = Aib
HXQGTFTSDYSKYLDEKKAKEFVEWLESA

SEQ ID NO: 135 - protein, 30 aa, synthetic construct
  MOD_RES 2: note = Aib
HXQGTFTSDYSKYLDEKKAKEFVEWLEAGG

SEQ ID NO: 136 - protein, 29 aa, synthetic construct
  MOD_RES 2: note = Aib
HXQGTFTSDYSKYLDEKKAKEFVEWLLQT

SEQ ID NO: 137 - protein, 29 aa, synthetic construct
  MOD_RES 2: note = Aib
HXQGTFTSDYSKYLDEKKAKEFVEWLLKT

SEQ ID NO: 138 - protein, 36 aa, synthetic construct
  MOD_RES 2: note = Aib
HXQGTFTSDYSKYLDEKKAKEFVEWLLNTGRNRNIA

SEQ ID NO: 139 - protein, 31 aa, synthetic construct
  MOD_RES 2: note = Aib
HXQGTFTSDYSKYLDEKKAKEFVEWLVKGRG

SEQ ID NO: 140 - protein, 29 aa, synthetic construct
  MOD_RES 2: note = Aib
HXQGTFTSDYSKYLDEKKAKEFVEWLLNT

SEQ ID NO: 141 - protein, 36 aa, synthetic construct
  MOD_RES 2: note = Aib
HXQGTFTSDYSKYLDEKKAKEFVEWLMNTKRNRNIA

SEQ ID NO: 142 - protein, 29 aa, synthetic construct
  MOD_RES 2: note = Aib
HXQGTFTSDYSKYLDEKKAKEFVEWLLDT

SEQ ID NO: 143 - protein, 9 aa, synthetic construct
YAINWVRQA

SEQ ID NO: 144 - protein, 9 aa, synthetic construct
IFGIANYAQ

SEQ ID NO: 145 - protein, 9 aa, synthetic construct [sequence omitted]

SEQ ID NO: 146 - protein, 252 aa, synthetic construct
EVQLVESGGGLVKPGGSLRLSCAASGFPFSSAWMSWVRQAPGKGLEWVGRIKSKADAGTTDYGAPVKGRFTISRDDSKNTLYLQMNSLKTEDTAVYYCTTEDIVGVPFKGYYQYMDAWGKGTTVTVSSSSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIK

SEQ ID NO: 147 - protein, 252 aa, synthetic construct; identical to SEQ ID NO: 146

SEQ ID NO: 148 - protein, 466 aa, synthetic construct
MTTSPILQLLLRLSLCGLLLQRAETGSKGQTAGELYQRWERYRRECQETLAAAEPPSGLACNGSFDMYVCWDYAAPNATARASCPWYLPWHHHVAAGFVLRQCGSDGQWGLWRDHTQCENPEKNEAFLDQRLILERLQVMYTVGYSLSLATLLLALLILSLFRRLHCTRNYIHINLFTSFMLRAAAILSRDRLLPRPGPYLGDQALALWNQALAACRTAQIVTQYCVGANYTWLLVEGVYLHSLLVLVGGSEEGHFRYYLLLGWGAPALFVIPWVIVRYLYENTQCWERNEVKAIWWIIRTPILMTILINFLIFIRILGILLSKLRTRQMRCRDYRLRLARSTLTLVPLLGVHEVVFAPVTEEQARGALRFAKLGFEIFLSSFQGFLVSVLYCFINKEVQSEIRRGWHHCRLRRSLGEEQRQLPERAFRALPSGSGPGEVPTSRGLSSGTLPGPGNEASRELESYC

SEQ ID NO: 149 - protein, 48 aa, synthetic construct
  MOD_RES 2: note = Aib
  MOD_RES 48: note = acetyl bromide
HXEGTFTSDVSSYLEEQAAKEFIAWLVKGRGGGGGSGGGGSGGGGSKX

SEQ ID NO: 150 - protein, 48 aa, synthetic construct
  MOD_RES 2: note = Aib
  MOD_RES 16: note = Aib
  MOD_RES 48: note = acetyl bromide
HXEGTFTSDVSSYLEXQAAKEFIAWLVKGRGGGGGSGGGGSGGGGSKX

SEQ ID NO: 151 - protein, 48 aa, synthetic construct
  MOD_RES 2: note = Aib
  MOD_RES 48: note = acetyl bromide
HXEGTFTSDYSSYLEEQAAKEFIAWLVKGRGGGGGSGGGGSGGGGSKX

SEQ ID NO: 152 - protein, 48 aa, synthetic construct
  MOD_RES 2: note = Aib
  MOD_RES 16: note = Aib
  MOD_RES 48: note = acetyl bromide
HXEGTFTSDYSSYLEXQAAKEFIAWLVKGRGGGGGSGGGGSGGGGSKX

SEQ ID NO: 153 - protein, 48 aa, synthetic construct
  MOD_RES 2: note = Aib
  MOD_RES 48: note = acetyl bromide
HXEGTFTSDVSSYLEEQAAKEFIAWLVKGGGGGGGSGGGGSGGGGSKX

SEQ ID NO: 154 - protein, 48 aa, synthetic construct
  MOD_RES 2: note = Aib
  MOD_RES 48: note = acetyl bromide
HXEGTFTSDYSSYLEEQAAKEFIAWLVKGGGGGGGSGGGGSGGGGSKX

SEQ ID NO: 155 - DNA, 384 bp, synthetic construct
  source 1..384: mol_type = other DNA
gaggtgcagctcgtggaaagcggaggaggcctggtgaaacctggcggctccctgcggctgagctgcgccgcttctggcttccccttcagcagcgcctggatgagctgggtcagacaggccccaggcaaaggcctggaatgggtgggccggatcaagtctaaggccgacgccggcacaacagactacggcgcccctgtgaagggaagattcaccatcagcagagatgactctaagaacaccctgtacctgcaaatgaacagcctgaagaccgaggacaccgccgtgtactattgtaccacagaggatatcgtgggcgtgccttttaagggctactaccagtacatggacgcttggggcaagggcaccaccgtgacagtgtccagc

SEQ ID NO: 156 - DNA, 1374 bp, synthetic construct
  source 1..1374: mol_type = other DNA
gaggtgcagctcgtggaaagcggaggaggcctggtgaaacctggcggctccctgcggctgagctgcgccgcttctggcttccccttcagcagcgcctggatgagctgggtcagacaggccccaggcaaaggcctggaatgggtgggccggatcaagtctaaggccgacgccggcacaacagactacggcgcccctgtgaagggaagattcaccatcagcagagatgactctaagaacaccctgtacctgcaaatgaacagcctgaagaccgaggacaccgccgtgtactattgtaccacagaggatatcgtgggcgtgccttttaagggctactaccagtacatggacgcttggggcaagggcaccaccgtgacagtgtccagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggaaaa

SEQ ID NO: 157 - DNA, 321 bp, synthetic construct
  source 1..321: mol_type = other DNA
gacatccagatgacccagagccccagcagcctgtctgccagcgtgggcgacagagtgaccatcacctgtagagccagccagagcatcagcagctacctgaactggtatcagcagaagcccggcaaggcccccaaactgctgatctacgccgccagctctctgcagtctggcgtgcccagcagattttccggcagcggctctggcaccgacttcaccctgaccatcagctccctgcagcccgaggacttcgccacctactactgccagcagagctacagcacccccctgacctttggccagggcaccaaggtggaaatcaaa

SEQ ID NO: 158 - DNA, 642 bp, synthetic construct
  source 1..642: mol_type = other DNA
gacatccagatgacccagagccccagcagcctgtctgccagcgtgggcgacagagtgaccatcacctgtagagccagccagagcatcagcagctacctgaactggtatcagcagaagcccggcaaggcccccaaactgctgatctacgccgccagctctctgcagtctggcgtgcccagcagattttccggcagcggctctggcaccgacttcaccctgaccatcagctccctgcagcccgaggacttcgccacctactactgccagcagagctacagcacccccctgacctttggccagggcaccaaggtggaaatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 159 - DNA, 360 bp, synthetic construct
  source 1..360: mol_type = other DNA
caggttcagctggtgcagagcggtgcagaagtgaaaaaacctggtagcagcgtgaaagtgtcatgcaaggcgtcaggctatacattctcatcatatgcaatcaactgggtcagacaggcgcccggacagggactggaatggatggggggcattgtgccgattttcggaattgcaaactatgcgcagaaatttcagggtcgtgtgaccatcactgcagatgaatcaaccagcaccgcctatatggaactgagcagcctgcgtagcgaagataccgcagtatactattgcgcccgtggaagtgattatgtggtcccccacttcgactattggggccaaggtacactggtcaccgtcagcagc

SEQ ID NO: 160 - DNA, 1350 bp, synthetic construct
  source 1..1350: mol_type = other DNA
caggttcagctggtgcagagcggtgcagaagtgaaaaaacctggtagcagcgtgaaagtgtcatgcaaggcgtcaggctatacattctcatcatatgcaatcaactgggtcagacaggcgcccggacagggactggaatggatggggggcattgtgccgattttcggaattgcaaactatgcgcagaaatttcagggtcgtgtgaccatcactgcagatgaatcaaccagcaccgcctatatggaactgagcagcctgcgtagcgaagataccgcagtatactattgcgcccgtggaagtgattatgtggtcccccacttcgactattggggccaaggtacactggtcaccgtcagcagcgcgtcgaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaagccgctggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggaaaa

SEQ ID NO: 161 - DNA, 330 bp, synthetic construct
  source 1..330: mol_type = other DNA
cagagcgtgctgacacagcctcccagcgcctctggcacccctggccagagagtgaccatcagctgcagcggatccagcagcaacatcggcagcaactacgtgtactggtatcagcaattgcccggcaccgcccccaagctgctgatctaccggaacaaccagcggcccagcggcgtgcccgatcggtttagcggcagcaagagcggcacaagcgccagcctggccatcagcggcctgagaagcgaggacgaggcggattactactgcgccgcctgggacgacagcctgagcggcgtggtgtttggaggcggcaccaagctgaccgtgcta

SEQ ID NO: 162 - DNA, 648 bp, synthetic construct
  source 1..648: mol_type = other DNA
cagagcgtgctgacacagcctcccagcgcctctggcacccctggccagagagtgaccatcagctgcagcggatccagcagcaacatcggcagcaactacgtgtactggtatcagcaattgcccggcaccgcccccaagctgctgatctaccggaacaaccagcggcccagcggcgtgcccgatcggtttagcggcagcaagagcggcacaagcgccagcctggccatcagcggcctgagaagcgaggacgaggcggattactactgcgccgcctgggacgacagcctgagcggcgtggtgtttggaggcggcaccaagctgaccgtgctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca